(19) United States
(12) Patent Application Publication
(10) Pub. No.: US 2008/0063607 A1
(43) Pub. Date: Mar. 13, 2008

(54) Oleaginous pharmaceutical and cosmetic foam

(75) Inventors: Dov Tamarkin, Maccabim (IL); Doron Friedman, Karmei Yosef (IL); Meir Eini, Ness Ziona (IL); Alex Besonov, Rehovot (IL); Jorge Danziger, Rishom Lezion (IL); David Schuz, Moshav Gimzu (IL); Tal Berman, Rishon LeZiyyon (IL)

Correspondence Address: WILMERHALE/BOSTON, 60 STATE STREET, BOSTON, MA, 02109, US

(73) Assignee: FOAMIX LTD

(21) Appl. No.: 11/894,798

(22) Filed: Aug. 20, 2007

Related U.S. Application Data
(63) Continuation of application No. 11/653,205, filed on Jan. 12, 2007.
(63) Continuation-in-part of application No. 10/911,367, filed on Aug. 4, 2004.
(63) Continuation-in-part of application No. 11/430,599, filed on May 9, 2006.
Application No. 11/430,599 is a continuation-in-part of application No. 10/835,505, filed on Apr. 28, 2004.
(60) Provisional application No. 60/492,385, filed on Aug. 4, 2003.
(60) Provisional application No. 60/492,385, filed on Aug. 4, 2003.
(60) Provisional application No. 60/530,015, filed on Dec. 16, 2003.
(60) Provisional application No. 60/679,020, filed on May 9, 2005.
(60) Provisional application No. 60/784,793, filed on Mar. 21, 2006.

(51) Int. Cl. A61K 9/12; A61K 47/00; A61P 17/00

(52) U.S. Cl. 424/43; 514/789

(57) ABSTRACT

The invention relates to stable pharmaceutical or cosmetic foam compositions containing certain active agents, having unique therapeutic properties and methods of treatment using such compositions. The foamable composition includes at least one solvent comprising polyethylene glycol (PEG) or PEG derivative and mixtures thereof, or comprising propylene glycol, wherein the solvent is present at a concentration of about 70% to about 96.5% by weight of the total composition, at least a non-ionic surface-active agent at a concentration of about 0.1% to less than about 10% by weight of the total composition.

### RELATED APPLICATIONS

This application claims the benefit of priority under 35 U.S.C. §120 to co-pending U.S. application Ser. No. 11/653,205, filed Jan. 12, 2007, entitled “Oleaginous Pharmaceutical and Cosmetic Foam, which is a continuation-in-part application of co-pending U.S. patent application Ser. No. 10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” and both hereby incorporated in their entirety by reference.

This application is a continuation-in-part application of co-pending U.S. patent application Ser. No. 10/835,505, filed on Apr. 28, 2004, which claims the benefit of priority under 35 U.S.C. §119(e) to U.S. Patent Application Ser. No. 60/530,015, filed on Dec. 16, 2003, both entitled “Oleaginous Pharmaceutical Foam” and both hereby incorporated in their entirety by reference.

### FIELD OF THE INVENTION

The invention relates to foam compositions including cosmetic or therapeutic active agents, and methods of topical treatment using the compositions.

### BACKGROUND OF THE INVENTION

Certain foam products for topical application of therapeutical agents and cosmetics have been prepared as oil-in-water emulsions. Foams and, in particular, foam compositions having a high oil content are complicated systems that do not form under all circumstances. Slight shifts in foam composition, such as the addition of an active ingredient, may destabilize the foam. It is known in the art that hydrophobic solvents are difficult to formulate into a foam-producing product. Addition of conventional hydrophobic solvents interferes with the foam forming ability of the surfactant, and thus, in the few foam products containing high-oil concentrations that have been reported, high surfactant concentrations are used, which may cause undesirable irritancy on one hand, and costly raw material usage on the other hand are used.

Oleaginous formulations for the preparation of cosmetic and therapeutic compositions are known in the art.

U.S. Pat. No. 6,620,773 relates to a foaming oil composition, which includes a surfactant mixture and an oil component, the surfactant mixture containing an anionic or zwitterionic surfactant, a nonionic surfactant and at least one ethoxylated alkyl phosphate ester component. The surfactant mixture ranges from about 15% to about 50% of the total composition, and that of the oil component ranges from about 50% to about 85%.

U.S. Pat. Nos. 5,700,396 and 5,589,515 disclose a cosmetic emulsion composition containing 1 to 99 wt % oily component (balance aqueous component). The oily component includes 85% or more weight % of cis Δ9-octadecanoic acid or derivatives thereof, which serves as a surfactant in the formulation.

U.S. Pat. No. 6,524,594 describes a gelled oil composition containing an emulsifier, a gelling agent, an oil, and a surfactant which, when applied to the skin in the presence of water, produces a significant amount of foam. The surfactant is used in an amount from about 10% to about 20%, and more preferably, from about 15% to about 20%.

U.S. Pat. No. 6,121,210 discloses foamable, silicone oil compositions and methods of lubricating surfaces with such compositions. The compositions are oil-in-water emulsions comprising silicone oil-in-water emulsion, a liquid propellant and a foam builder comprising a solid, non-ionic lipophilic surfactant having an HLB value of about 3 to about 8. Foam stabilizers including long claim fatty alcohols are included. A propellant is included to create a foamable composition.

In general, the foamable compositions of the art are based on oil-in-water emulsions. Furthermore, they often include a high content level of surfactants and foaming agents required to form acceptable foams which are stable and possess low specific gravity. Such surfactants, and particularly ionic surfactants, such as anionic surfactants (e.g. sodium lauryl sulfate (SDS)), may have adverse affects on certain patients, including concentration-dependent skin irritation.

There remains an unmet need for improved, stable and non-irritating foam formulations and oleaginous foam formulations, intended for dermal and mucosal delivery of pharmaceutical and cosmetic, with unique therapeutic and cosmetic properties.

### SUMMARY OF THE INVENTION

The invention relates to stable pharmaceutical or cosmetic foam compositions containing certain active agents, having unique therapeutic properties and methods of treatment using such compositions. The foamable composition includes at least one solvent comprising polyethylene glycol (PEG) or PEG derivative and mixtures thereof, or comprising propylene glycol, wherein the solvent is present at a concentration of about 70% to about 96.5% by weight of the total composition, at least a non-ionic surface-active agent at a concentration of about 0.1% to less than about 10% by weight of the total composition.

The present invention provides stable foam-forming compositions, and stable oleaginous foam-forming compositions including at least one active agent for dermal and mucosal delivery. The composition is dispensed as a foam providing a stable product that is pleasant and easy to spread, resulting in high patient compliance. The invention more particularly relates to a foamable pharmaceutical or cosmetic composition, comprising:

a solvent comprising polyethylene glycol (PEG) or PEG derivative and mixtures thereof, wherein the PEG or PEG derivative is present at a concentration of about 70% to about 96.5% by weight of the total composition;

a surface-active agent at a concentration of about 0.1% to less than about 10% by weight of the total composition; and

a therapeutically effective amount of at least one active agent.

The invention also relates to a foamable pharmaceutical or cosmetic carrier composition for dermatological use, comprising:

a solvent comprising polyethylene glycol (PEG) or PEG derivative and mixtures thereof, wherein the PEG or PEG derivative is present at a concentration of about 70% to about 96.5% by weight of the total composition; and

a surface-active agent at a concentration of about 0.1% to less than about 10% by weight of the total composition.

at least one gelling agent at a concentration of about 0.1% to about 5% by weight of the total composition.

at least one liquefied or compressed gas propellant, at a concentration of about 3% to about 25% by weight of the total composition

wherein the composition is stored in an aerosol container and upon release expands to form a breakable foam.

The invention further relates to a composition, comprising:

a polyethylene glycol (PEG) or PEG derivative and mixtures thereof, wherein the PEG or PEG derivative is present at a concentration of about 70% to about 96.5% by weight of the total composition;

a surface-active agent at a concentration of about 0.1% to less than about 10% by weight of the total composition; and

a therapeutically effective amount of a agent, an antinfective agent, a keratolytically active agent, a vasoactive agent and a retinoid.

The invention further relates to a composition, comprising:

a polyethylene glycol (PEG) or PEG derivative and mixtures thereof, wherein the PEG or PEG derivative is present at a concentration of about 70% to about 96.5% by weight of the total composition;

a surface-active agent at a concentration of about 0.1% to less than about 10% by weight of the total composition; and

a therapeutically effective amount of a an anti-inflammatory or antiallergic agent, wherein said agent reduces the occurrence of pro-inflammatory cytokines or inhibits the effect of pro-inflammatory cytokines.

The invention still further relates to a foamable pharmaceutical or cosmetic composition, comprising:

a solvent comprising propylene glycol, wherein the propylene glycol is present at a concentration of about 70% to about 96.5% by weight of the total composition;

a surface-active agent at a concentration of about 0.1% to less than about 10% by weight of the total composition; and

a therapeutically effective amount of at least one active agent.

The invention still further relates to a foamable pharmaceutical or cosmetic carrier composition for dermatological use, comprising:

a solvent comprising propylene glycol, wherein the propylene glycol is present at a concentration of about 70% to about 96.5% by weight of the total composition; and

a surface-active agent at a concentration of about 0.1% to less than about 10% by weight of the total composition.

at least one gelling agent at a concentration of about 0.1% to about 5% by weight of the total composition.

at least one liquefied or compressed gas propellant, at a concentration of about 3% to about 25% by weight of the total composition

wherein the composition is stored in an aerosol container and upon release expands to form a breakable foam.

The invention still further relates to a composition, comprising:

a solvent comprising propylene glycol, wherein the propylene glycol is present at a concentration of about 70% to about 96.5% by weight of the total composition;

a surface-active agent at a concentration of about 0.1% to less than about 10% by weight of the total composition; and

a therapeutically effective amount of a agent, an antinfective agent, a keratolytically active agent, a vasoactive agent and a retinoid.

The invention still further relates to a composition, comprising:

a solvent comprising propylene glycol, wherein the propylene glycol is present at a concentration of about 70% to about 96.5% by weight of the total composition;

a surface-active agent at a concentration of about 0.1% to less than about 10% by weight of the total composition; and

a therapeutically effective amount of a an anti-inflammatory or antiallergic agent, wherein said agent reduces the occurrence of pro-inflammatory cytokines or inhibits the effect of pro-inflammatory cytokines.

The invention also relates to a method of treating, alleviating or preventing a dermatological, cosmetic or mucosal disorder, comprising administering topically to a subject having said disorder a therapeutically effective amount of any of the compositions, foamable compositions and foam compositions described herein.

In the context of the present invention, an oleaginous foam is a composition comprising at least one solvent selected from a hydrophobic solvent, a co-solvent, an emollient and mixtures thereof in the continuous phase of the composition. In specific embodiments, the foamable compositions and foams produced from them include a solvent comprising polyethylene glycol (PEG) or PEG derivative and mixtures thereof, or comprising polypropylene glycol.

According to one aspect or the present invention, the composition includes:

a. at least one solvent selected from a hydrophobic solvent, a co-solvent, and mixtures thereof, wherein the solvent is present at a concentration of about 70% to about 96.5% by weight of the total composition;

b. a non-ionic surface-active agent at a concentration of about 0.1% to less than about 10% by weight of the total composition;

c. at least one gelling agent at a concentration of about 0.1% to about 5% by weight of the total composition;

d. at least one active agent in a therapeutically effective concentration; and

e. at least one liquefied or compressed gas propellant, at a concentration of about 3% to about 25% by weight of the total composition.

Water and optional ingredients are added to complete the total weight to 100%, although the composition may be essesntially free of lower alkyl alcohols. In one or more embodiments, the oleaginous composition of the present invention contains less than about 5% of a lower alcohol having up to 5 carbon atoms in its carbon chain skeleton.

In one or more embodiments, the oleaginous composition includes water at a concentration less than about 30%, preferably less than about 20%, more preferably less than about 10% by weight.

In one or more embodiments, the oleaginous composition of the present invention further includes a foam adjuvant.

In yet other embodiments, the oleaginous composition of the present invention forms an emulsion.

In one or more embodiments, the oleaginous composition of the present invention includes a hydrophobic solvent having solubility in distilled water at ambient temperature of less than about one gram per 100 ml. The hydrophobic solvent may be a mineral oil, MCT oil, triglyceride oil, silicone oil, a polyunsaturated oil, an unsaturated oil and an essential oil, and mixtures thereof.

In one or more embodiments, the at least one solvent is a co-solvent. In one or more embodiments, the co-solvent is a polyethylene glycol derivative, or glycerin. In one or more embodiments, the oleaginous composition of the present invention includes a mixture of at least one hydrophobic solvent and at least one co-solvent. The mixture of at least one hydrophobic solvent and the at least one co-solvent may have a weight ratio of about 1:8 to about 8:1. In one or more embodiments, a mixture of at least one hydrophobic solvent and glycerin is used; and the mixture may have a weight ratio of about 1:4 to about 4:1, or about 1:2 to about 2:1.

According to one or more embodiments, the composition includes at least one solvent having a high solubilization capacity, termed herein a “potent solvent”. In the context of the present invention, a potent solvent is other than mineral oil and solubilizes a specific active agent substantially better than a hydrocarbon solvent such as mineral oil or petrolatum, for example, 5-fold better than mineral oil; or even 10-fold better than mineral oil.

In one or more embodiments, the oleaginous composition of the present invention contains a potent solvent selected from the group consisting of polyethylene glycol, propylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol).

In one or more embodiments, the surface-active agent is a non-ionic surfactant and can be, for example, a phospholipid. The surface-active agent can be a mixture of at least one non-ionic surfactant and at least one ionic surfactant, for example, at a weight ratio of about 20:1 to about 1:1.

In one or more embodiments, the composition includes at least one gelling agent selected from the group consisting of natural polymeric materials, semi-synthetic polymeric materials, synthetic polymeric materials, inorganic gelling agents and mixtures thereof.

The oleaginous composition of the present invention upon extrusion from a pressured container has a specific gravity of about 0.02 g/mL to about 0.5 g/mL, and is useful for treating, alleviating or preventing a dermatological or mucosal disorder.

According to a further aspect of the present invention, an oleaginous water-in-oil emulsion is provided. The emulsion can be essentially free of lower alkyl alcohols. The emulsion includes:

at least one solvent selected from a hydrophobic solvent, a co-solvent and an emollient at a concentration of about 30% to about 96.5% by weight;

water;

at least one non-ionic lipophilic surface acting agent having an HLB value of about 3 to about 10 at a concentration of about 0.1% to less than about 10% by weight,

at least one gelling agent at a concentration of about 0.1% to about 5% by weight.

at least one active agent at a therapeutically effective concentration; and

at least one liquefied or compressed gas propellant at a concentration of about 3% to about 25% by weight of the total composition.

In one or more embodiments, the oleaginous emulsion of the present invention contains less than about 5% of a lower alcohol having up to 5 carbon atoms in its carbon chain skeleton. In another embodiment the oleaginous composition of the present invention further comprises a foam adjuvant.

In one or more embodiments, the oleaginous water-in-oil emulsion contains a hydrophobic solvent and water at a weight ratio of about 1:3 to about 6:1.

In one or more embodiments, the oleaginous emulsion contains a hydrophobic solvent having solubility in distilled water at ambient temperature of less than about one gram per 100 ml. The hydrophobic solvent may be selected from mineral oil, MCT oil, triglyceride oil, silicone oil, a polyunsaturated oil, an unsaturated oil and an essential oil.

The oleaginous emulsion may include a potent solvent selected from a hydrophobic solvent other than mineral oil, a co-solvent and an emollient, wherein the potent solvent solubilizes the active agent substantially better than mineral oil solubilizes the active agent, e.g at least 5-fold better or at least 10-fold better than mineral oil solubilizes the active agent.

In one or more embodiments, the oleaginous emulsion contains a surface-active agent having a HLB value in the range of about 3 to about 10, which promote the formation of a water-in-oil emulsion.

In one or more embodiments, the oleaginous emulsions contains at least one gelling agent selected from the group consisting of natural polymeric materials, semi-synthetic polymeric materials, synthetic polymeric materials, inorganic gelling agents and mixtures thereof.

The active agent can be a therapeutic agent or a cosmetic agent. The therapeutic agent is selected for the treatment or prophylaxis of a disorder of the skin, mucosal membrane, ear channel, vagina, penile urethra and rectum. In one embodiment therapeutic agent is selected from an anti-infective, an antibiotic, an antibacterial agent, an antifungal agent, an antiviral agent, an antiparasitic agent, an antiinflammatory agent, an anesthetic, an analgesic, an antiallergic agent, a corticosteroid, a retinoid, an antiproliferative agent, an anticancer agent, a photodynamic therapy agent, a lubricating agent and mixtures thereof.

Alternatively, the active agent is an inorganic solid matter, preferably a metal oxide, more preferably zinc oxide.

The active agent can also be a cosmetic agent such as a retinoid, an anti-wrinkle agent, a radical scavenger, a self-tanning agent, a skin whitening agent a skin protective agent, an anti-cellulite agent, a massaging oil and an anti-wart agent.

In another aspect, the present invention provides a method of treating, alleviating or preventing a dermatological or mucosal disease or disorder, comprising administering topically to a subject having the disease or disorder a therapeutically effective amount of the oleaginous compositions or the oleaginous water-in-oil emulsions of the present invention.

In yet another aspect, the present invention also provides a method of designing a foamable composition, containing at least one active agent that is substantially insoluble in a hydrocarbon solvent including mineral oil. The method includes selecting at least one active agent, and identifying a solvent that solubilizes the active agent substantially better than mineral oil solubilizes the active agent. The method may further comprise the step of adjusting the type and concentration of surfactant and gelling agent to provide a foamable composition.

In one or more embodiments, the potent solvent solubilizes the active agent 5-fold better or even 10-fold better than mineral oil solubilizes the active agent.

### DETAILED DESCRIPTION OF THE INVENTION

Despite the commonly known fact that hydrophobic solvents, and oils in particular, are difficult to formulate into foam-producing products and that addition of conventional hydrophobic solvents interferes with the foam forming ability of the surfactant, the present invention has surprisingly discovered stable oleaginous foam compositions, comprising at least one active agent for dermal and mucosal delivery. The compositions are dispensed as a foam providing a stable product that is pleasant and easy to use for high patient and consumer compliance. The at least one active agent is selected from a therapeutically active agent or a cosmetic agent.

Surprisingly, the compositions of the present invention require low surfactant concentrations, e.g., less than 10% by weight and often much less, thus preventing both undesirable irritancy and costly raw material usage.

According to one aspect of the present invention, the foamable compositions are light weight, have low density, spread easily and comfortably over large body area, and are thus, economical.

The compositions of the present invention comprise at least one solvent selected from a hydrophobic solvent, a co-solvent, an emollient and mixtures thereof, which provides a refatting and skin soothing effect. The selected solvents allow the inclusion of oil-soluble active agents in the formulation. In one or more embodiments, the solvents provide synergistic benefits in combination with the active agent. The compositions may comprise at least one oil soluble active agent.

In one or more embodiments, the compositions require only low concentrations of a foaming agent in order to generate a stable foam. The reduced surfactant requirement is advantageous since surfactants are known to be irritating when in contact with the skin at elevated concentrations.

The compositions are easily spreadable, allowing treatment of large areas such as the arms, back, trunk, legs and the breast. Furthermore, due to flow properties, they spread effectively into folds and wrinkles and absorb into the skin, providing uniform distribution of the active agent without the need of extensive rubbing thus providing a unique means for the treatment of large body areas.

The compositions may be used for the treatment of body cavities, such as the vagina, penile urethra, rectum and the ear channel due to their expansion properties.

Class A foam composition

According to one aspect the present invention provides an oleaginous foam composition for topical application including:

at least one solvent selected from a hydrophobic solvent, a co-solvent, an emollient and mixtures thereof, at a concentration of about 70% to about 96.5% by weight,

at least a non-ionic surface active agent at a concentration of about 0.1% to less than about 10% by weight and, optionally, having an HLB value of about 9 or less;

at least one gelling agent at a concentration of about 0.1% to about 5% by weight;

at least one active agent at a therapeutically effective concentration; and

a liquefied or compressed gas propellant at a concentration of about 3% to about 25% by weight of the total composition.

The balance of the composition contains water and additional optional components. The content of the foam composition is presented herein as concentration (percent by weight, % w/w). The foam composition can be a homogeneous mixture or an emulsion. confirm that this is true for the Class A foams.

Such a composition is placed in a pressurized aerosol container and, upon release from the container, creates a novel therapeutically-beneficial foam product.

Low water content is important in order to attain high skin and body tissue lubrication, refatting, occlusive effects and effective skin absorption of a active agents. It is also important in order to avoid degradation of water sensitive active agents.

Thus, in one or more embodiments, the composition comprises water at a concentration of about 30% or less, or at a concentration less than about 20%, or at a concentration less than about 10% by weight.

The composition is optionally substantially free of short chain alcohols, i.e. comprises less than about 5% by weight of a short chain alcohol having 5 or less carbon atom in its skeleton, and may further comprise a foam adjuvant.

According to one embodiment, the composition comprises a solvent selected from a hydrophobic solvent and an emollient and at least one co-solvent. According to one embodiment the co-solvent is a hydrophilic solvent, other than a short chain alcohol, selected from an organic solvent that dissolves in water. Non-limiting examples of such co-solvents include propylene glycol, glycerol, and other poly-hydroxy solvents. Preferably, the composition comprises glycerol as co-solvent. In one embodiment the composition comprises a hydrophobic solvent component and a co-solvent at a weight ratio in the range of about 4:1 and about 1:4, or about 2:1 to 1:2. In an even further embodiment of the present invention, the co-solvent constitutes a continuous phase of the emulsion and a minor portion of water is included in the co-solvent phase.

Such a composition is placed in an aerosol container and, upon release from the aerosol container, creates a therapeutically-beneficial foam product.

Class B foam composition

According to another aspect the present invention provides an oleaginous foam composition comprising water-in-oil emulsion, i.e., an emulsion having one phase comprising at least one hydrophobic component (oil phase) and one phase which comprises water. Due to the fact that the continuous phase of the emulsion is the oil phase, the composition provides oily feeling, occlusive properties and protective effects. Notably, while it is known that a composition with a continuous oil phase is unlikely to form foam without high amounts of surfactants, the composition of the present invention surprisingly forms a stable foam with low density. In one or more embodiments, there is an overlap between the compositions of Class A and Class B, the distinction being that Class B compositions are formed as water-in-oil emulsions.

According to one embodiment, the water-in-oil emulsion composition contains:

at least one solvent selected from a hydrophobic solvent, a co-solvent, an emollient and mixtures thereof, at a concentration of about 30% to about 96% by weight,

water at a concentration of 1% to about 70% by weight;

at least one non-ionic lipophilic surface active agent, preferably having an HLB value of about 3 to about 10, more preferably about 3.5 to about 9 at a concentration of about 0.1% to about 10% by weight, or between about 0.1% and about 5% by weight, or even between about 0.1% and about 2% by weight;

at least one gelling agent at a concentration of about 0.1% to about 5% by weight;

at least one active agent at a therapeutically effective concentration; and a liquefied or compressed gas propellant at a concentration of about 3% to about 25% by weight of the total composition, in an aerosol container.

According to a further embodiment, the ratio between the oil phase and water is between about 1:3 and about 6:1.

The term “oleaginous” is defined as “having the nature or qualities of oil”. The terms “oleaginous composition”, “oleaginous foam” and “oleaginous foamable composition” as used herein interchangeably refer to a composition that has the organoleptic character of an oily substance, i.e., oily feeling, when topically administered to a body area, such as the skin or mucosal tissue.

In the context of the present invention, an oleaginous foam is a composition comprising at least one solvent selected from a hydrophobic solvent, a co-solvent, an emollient and mixtures thereof in the continuous phase of the composition and is characterized by an oily feeling upon application to a body surface.

Such an oleaginous composition may provide an enhanced occlusive effect, which may in turn control the drug residence time and skin penetration of an active agent. Furthermore, oleaginous compositions provide moisturizing effects, refatting effects, protective effects and lubrication which contribute to the treatment of dermatological disorders. Thus, a composition of this nature, comprising an oleaginous vehicle and an active agent is expected to provide a synergistic therapeutic effect.

Solvents

At least one solvent of the composition of the present invention is selected from a hydrophobic solvent, an emollient, a silicone oil, a co-solvent, and a mixture thereof. The solvent occupies at least the continuous phase; however, it may also partition into the discontinuous phase in those instances when the composition is an emulsion.

Hydrophobic Solvent

A “hydrophobic solvent” as used herein refers to a material having solubility in distilled water at ambient temperature of less than about 1 gm per 100 mL, or less than about 0.5 gm per 100 mL, or even less than about 0.1 gm per 100 mL. It is liquid at ambient temperature.

In one preferred embodiment, the at least one solvent is a hydrophobic solvent such as mineral oil. Mineral oil (Chemical Abstracts Service Registry number 8012-95-1) is a mixture of aliphatic, naphthalenic, and aromatic liquid hydrocarbons that derive from petroleum. They are typically liquid, their viscosity is in the range of between about 35 CST and about 100 CST (at 40° C.), and their pour point (the lowest temperature at which an oil can be handled without excessive amounts of wax crystals forming so preventing flow) is below 0° C. By contrast, white petrolatum, also termed “Vaseline”, is disadvantageous, due to its waxy nature and semi-solid texture. It is known to leave a waxy and sticky feeling after application and occasionally stain cloths. Thus, white petrolatum as well as other wax-like, semi-solid compounds are undesirable as a hydrophobic solvent according to the present invention.

According to one embodiment the oleaginous foam composition of the present invention comprises at least one solvent that is a hydrophobic solvent selected from mineral oil, a triglyceride oil, an ester of a fatty acid, an ester of a dicarboxylic acid, silicone oil, a polyunsaturated oil, an unsaturated oil and an essential oil.

According to one embodiment, preferred hydrophobic solvents are liquid oils originating from vegetable, marine or animal sources. The hydrophobic solvent may be selected from a saturated or an unsaturated oil. By way of example, the unsaturated oil may be selected from the group consisting of olive, corn, soybean, canola, cottonseed, coconut, sesame, sunflower, borage seed, syzigium aromaticum, hempseed, herring, cod-liver, salmon, flaxseed, wheat germ and evening primrose oils and mixtures thereof, at any proportion.

One class of hydrophobic solvents includes polyunsaturated oils, containing omega-3 and omega-6 fatty acids, which are know to possess therapeutic properties though different modes of action. Examples of such polyunsaturated fatty acids are linoleic and linolenic acid, gamma-linoleic acid (GLA), eicosapentaenoic acid (EPA) and docosahexaenoic acid (DHA). Thus, in one preferred embodiment of the present invention the at least one hydrophobic solvent comprises at least 6% of an oil selected from omega-3 oil, omega-6 oil, and mixtures thereof.

Another preferred class of hydrophobic solvents comprises the essential oils, which are considered “therapeutic oils”, which contain active biologically occurring molecules and, upon topical application, exert a therapeutic effect. Examples of such oils are rosehip oil, which contain retinoids and is known to reduce acne and post-acne scars, tea tree oil, which possesses anti-microbial activity including antibacterial, antifungal and antiviral properties. Other examples of essential oils are basil, camphor, cardamom, carrot, citronella, clary sage, clove, cypress, frankincense, ginger, grapefruit, hyssop, jasmine, lavender, lemon, mandarin, marjoram, myrrh, neroli, nutmeg, petitgrain, sage, tangerine, vanilla, verbena, as well as any other therapeutically beneficial oil known in the art of herbal medication.

Emollient

A further preferred class of solvents are “emollients” that have a softening, refatting, or soothing effect, especially when applied to body areas, such as the skin and mucosal surfaces. Emollients are not necessarily hydrophobic. Without derogating the generality of this definition, examples of suitable emollients for use include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof. Examples of other suitable emollients may be found in the Cosmetic Bench Reference, pp. 1.19-1.22 (1996).

Silicone oil

According to the present invention, silicone oils are particularly preferred solvents, due to their known skin protective and occlusive properties. Suitable silicone oils or fluids for use in the invention may be selected from non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are preferably chosen from cyclic or linear polydimethylsiloxanes containing from about 3 to about 9, preferably from about 4 to about 5, silicon atoms. Volatile silicones such as cyclomethicones can also be used. Water-soluble silicones, such as dimethicone copolyol are not included in the definition of silicone oils (as hydrophobic solvents) according to the present invention. In one or more embodiments, the at least one solvent comprises at least 2% silicone oil, or at least 5% silicone oil.

Co-Solvent

A “co-solvent” is an organic solvent, other than a short chain alcohol, typically soluble in both water and oil. Examples of co-solvents, according to the present invention include: polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide; monooleate of ethoxylated glycerides (with 8 to 10 ethylene oxide units); azone (1-dodecylazacycloheptan-2-one), 2-(n-nonyl)-1,3-dioxolane; esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one preferred embodiment the co-solvent is a polyethylene glycol (PEG) or PEG derivative that is liquid at ambient temperature, including PEG200 (MW about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

In one or more preferred embodiments, the at least one solvent comprises a mixture (e.g., an emulsion) of a hydrophobic solvent and glycerin, as described, for example, in U.S. Pat. No. 6,544,530 to Friedman. The ratio of hydrophobic solvent to glycerin can range from about 1:4 to about 4:1, and more preferably from about 1:2 to about 2:1.

In several cases, a given solvent can be defined as both emollient and co-solvent.

Potent solvent

In one or more embodiments of the present invention, the foamable composition includes a potent solvent, in addition to or in place of one of the hydrophobic solvents, co-solvents and emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments of the present invention, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition of the present invention; or even more than about 60%.

Non-limiting examples of pairs of active agent and potent solvent include:

Betamethasone valerate: Practically insoluble in mineral oil (<0.01%); soluble more than 1% in glycofurol.

Hydrocortisone butyrate: Practically insoluble in mineral oil (<0.01%); soluble more than 1% in glycofurol.

Metronidazole: Practically insoluble in mineral oil (<0.01%); soluble more than 1% in dimethyl isosrbide.

Ketoconazole: Practically insoluble in mineral oil (<0.01%); soluble more than 1% in glycofurol, propylene glycol and dimethyl isosrbide.

Mupirocin: Practically insoluble in mineral oil (<0.01%); soluble more than 1% in glycofurol, hexylene glycol, dimethyl isosorbide, propylene glycol and polyethylene glycol 400 (PEG 400).

Meloxicam, a nonsteroidal anti-inflammatory agent: Practically insoluble in mineral oil (<0.001%); soluble in propylene glycol: 0.3 mg/mL; and in PEG 400: 3.7 mg/mL.

Progesterone: Practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol).

In another aspect, the present invention provides a method of designing a stable oleaginous foamable composition by selecting at least one active agent; and identifying a solvent that solubilizes the active agent substantially better than mineral oil or petrolatum, for example, solubilizes the active agent 5-fold better or even 10-fold better than a hydrocarbon solvent such as mineral oil or petrolatum. The method may further include adjusting the type and concentration of surfactant and gelling agent to provide a foamable composition.

The use of a potent solvent in a foam composition provides an improved method of delivering poorly soluble therapeutic agents to a target area. It is known that low drug solubility results in poor bioavailability, leading to decreased effectiveness of treatment. Foam compositions of the present invention, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams of the present invention, which are drip-free, provide a superior vehicle for such active agents, enabling convenient usage and accurate effective dosing.

The at least one solvent of the present invention may include a mixture of the above solvents selected from the group of hydrophobic solvents, silicone oils, emollients co-solvents and potent solvents in any proportion.

Surface-Active Agents

Surface-active agents (surfactants) may include an agent that has a property selected from linking oil and water in the composition, in the form of an emulsion, and evolving a foam. A surfactant's hydrophilic/lipophilic balance (HLB) describes the emulsifier's affinity towards water or oil. The HLB scale ranges from about 1 (totally lipophilic) to 45 (totally hydrophlic) and in the case of non-ionic surfactants from 1 to 20 totally hydrophlic), with 10 representing an equal balance of both hydrophilic and lipophilic characteristics. Lipophilic emulsifiers from water-in-oil (w/o) emulsions, hydrophilic surfactants form oil-in-water (o/w) emulsions. The HLB of a blend of two emulsifiers equals the weight fraction of emulsifier A times its HLB value, plus the weight fraction of emulsifier B times its HLB value. (weighted average).

Without wishing to be bound by any particular theory or mode of operation, hydrophilic surfactants produce oil-in-water (o/w) microemulsions, whereas lipophilic surfactants are used to promote emulsification of the aqueous phase into the oil phase.

The composition of the present invention according to one or more embodiments includes at least one surface active agent or surfactant, which is intended to both stabilize the formulation and to evolve an acceptable foam.

A composition having a low concentration of an ionic surfactant is important in terms of safety, since high concentrations of surfactants are known to evolve skin and mucosal membrane irritation. Unlike certain foamable oleaginous compositions of the art, the total surfactant employed to obtain foam that is stable, of low specific gravity and has a fine bubble structure is relatively low. Low surfactant levels, particularly of ionic surfactants, are preferred to reduce skin irritations. The composition of the present invention comprises total surfactant in the range of about 0.1% to less than about 10% of the foamable composition, and is typically less than about 5%, or even less than about 2%.

According to one or more embodiments the at least one surfactant is selected from hydrophilic, hydrophobic, and a mixture of hydrophilic and hydrophobic surfactants. As is well known in the art, the terms “hydrophilic” and “hydrophobic” are relative terms. A combination of surface-active agents is possible.

According to one or more embodiments, suitable surfactants for formation of a water-in-oil emulsion have an HLB value of no greater than 10, preferably from about 3 to about 9. Thus, the composition may include a single surface-active agent having an HLB value between 3 and 9, or a mixture of surface-active agents having a weighted average of their HLB values between 3 and 9.

Suitable water-in-oil emulsifiers include, but are not limited to, sorbitan derivatives such as sorbitan laurate and sorbitan palmitate; alkoxylated alcohols such as laureth-4; hydroxylated derivatives of polymeric silicones, such as dimethicone copolyol; alkylated derivatives of hydroxylated polymeric silicones, such as cetyl dimethicone copolyol; glyceryl esters such as polyglyceryl-4 isostearate; beeswax derivatives such as sodium isostearoyl-2-lactylate; lecithin; and mixtures thereof. In conjunction with the oil component being a silicone oil, the preferred emulsifiers are hydroxylated derivatives of polymeric silicones and alkylated derivatives thereof.

According to one or more embodiments the present invention, the composition comprises at least one non-ionic surfactant. In one or more embodiments, the composition includes at least one non-ionic surfactant and at least one ionic surfactant selected from the group of anionic, cationic, zwitterionic, at a weight ratio of between about 1:1 and about 20:0.1, or preferably at a weight ratio of about 4:0.1 to about 20:0.1.

The choice of specific surfactants should be made keeping in mind the particular hydrophobic therapeutic agent to be used in the composition, and the range of polarity appropriate for the chosen therapeutic agent. With these general principles in mind, a very broad range of surfactants is suitable for use in the present invention.

Additional non-limiting examples of possible surfactants include polysorbates, such as polyoxyethylene (20) sorbitan monostearate (Tween 60) and polyoxyethylene (20) sorbitan monooleate (Tween 80); Polyoxyethylene (POE) fatty acid esters, such as Myrj 45, Myrj 49 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; fatty alcohols or acids, mono or diglycerides, isoceteth-20, sodium methyl cocoyl taurate, sodium methyl oleoyl taurate, sodium lauryl sulfate, triethanolamine lauryl sulfate and betaines, provided that, in the case of a single surfactant, the HLB value is between 3 and 9; and in the case of a mixture of surface-active agents, the weighted average of their HLB values is between 3 and 9.

In one or more embodiments, the at least one surface active agent is a phospholipid. In a one or more embodiments, the phospholipid is phosphatidylcholine or 1,2-diacyl-sn-glycerol-3-phosphorylcholine, also termed “lecithin”, which is a naturally occurring phospholipid which possesses surfactant properties. Lecithin is the most abundant lipid in the membranes of biological tissues and as such, is considered a non-irritant. Lethicin is a phospholipid composition very similar in composition to that of human skin. For this reason, it is possible to use lethicin as an emulsifier or a surfact-active agent at levels about 10% by weight. In one or more embodiments, the surface-active agent includes lethicin up to about 10% by weight and the total surfact-active agent (when a mixture of agents is used) can be up to 15% by weight.

A composition having a low concentration of an ionic surfactant, preferably no ionic surfactant, is important in terms of safety, since high concentrations of surfactants are known to evolve skin irritation.

Gelling Agents

The composition according to one or more embodiments of the present invention include at least one gelling agent at a concentration of about 0.1% to about 5%. At least one gelling agent is selected from a natural polymeric material, a semi-synthetic polymeric material, a synthetic polymeric material, an inorganic gelling agent and mixtures thereof.

Exemplary gelling agents that can be used in accordance with one or more embodiments of the present invention include for example, but are not limited to, naturally-occurring polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

Yet, another preferred group of gelling agents includes inorganic gelling agents, such as silicone dioxide (fumed silica) including but not limited to AEROSIL 200 (DEGUSSA).

At least one gelling agent is present in an amount in the range of about 0.1% to about 5.0 wt % of the foamable composition. In one or more embodiments, it is typically less than 1 wt % of the foamable composition.

Foam Adjuvants

The composition of the present invention may optionally further include at least one foam adjuvant. In one or more embodiments, foam adjuvants include fatty alcohols having 15 or more carbons in their carbon chain, such as cetyl alcohol and stearyl alcohol (or mixtures thereof). Other examples of fatty alcohols are oleyl alcohol (C18, unsaturated), arachidyl alcohol (C20), behenyl alcohol (C22), 1-triacontanol (C30), as well as alcohols with longer carbon chains (up to C50). The concentration of the fatty alcohol that is required to support the foam system is inversely related to the length of its carbon chains. Fatty alcohols derived from beeswax including a mixture of alcohols, a majority of which has at least 20 carbon atoms in their carbon chain, are especially well suited as foam adjuvants according to the present invention.

Another class of foam adjuvants, according to one or more embodiments of the present invention, includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof.

Optionally, the carbon atom chain of the fatty alcohol or the fatty acid may have at least one double bond. A further class of foam adjuvant according to the present invention comprises a long chain fatty alcohol or fatty acid, wherein the carbon atom chain is branched. In an additional preferred class of foam adjuvants, the carbon chain of said fatty acid is substituted with a hydroxyl group, such as 12-hydroxy stearic acid.

The foam adjuvant according to the present invention may comprise a mixture of fatty alcohols, fatty acids and hydroxy fatty acids and derivatives thereof in any proportion, providing that the total concentration is about 0.1% to about 10% (w/w) preferably about 0.1% to about 5% (w/w) in one or more embodiments, the total concentration is about 0.4% to about 2.5% (w/w) of the total composition.

A feature of fatty alcohols and fatty acids relevant to their use in the foamable compositions according to one or more embodiments of the present invention is related to their therapeutic properties per se. Long chain saturated and mono unsaturated fatty alcohols, e.g., stearyl alcohol, erycyl alcohol, arachidyl alcohol and docosanol have been reported to possess antiviral, anti infective, anti-proliferative and anti-inflammatory properties (U.S. Pat. No. 4,874,794). Longer chain fatty alcohols, e.g., tetracosanol, hexacosanol, heptacosanol, octacosanol, triacontanol, etc. are also known for their metabolism modifying properties and tissue energizing properties. Long chain fatty acids have also been reported to possess anti-infective characteristics. Thus, the pharmaceutical or cosmetic composition of the present invention, comprising the optional foam adjuvant provides an extra or added therapeutic benefit.

Water Content

The creation of a foamable composition with low water content is not easy, and usually requires very high concentrations of a foaming surfactant system, which may comprise a high proportion of ionic surfactants. However, ionic surfactants are known to be skin irritants in a concentration-dependent manner, and thus, their use in the treatment of sensitive skin and other body tissues is very limited. Surprisingly, the compositions of the present invention have a low water content, and yet require very low concentration of surfactants, which are primarily non-ionic.

Substantially Alcohol Free

Lower alcohols, having up to 5 carbon atoms in their carbon chain skeleton, such as ethanol, propanol, isopropanol, butanol, iso-butanol, t-butanol and pentanol are considered less desirable solvents or co-solvents due to their skin-irritating effect. Thus, the composition of the present invention is substantially alcohol-free and should comprise less than about 5% final concentration of lower alcohols, preferably less than 2%, more preferably less than 1%.

Optional Ingredients

The pharmaceutical or cosmetic composition of the present invention may further optionally comprise a variety of therapeutic or cosmetic ingredients, which are added in order to fine-tune the consistency of the formulation, protect the formulation components from degradation and oxidation and bestow their cosmetic acceptability. Such excipients may be selected, for example, from the group consisting of diglycerides, triglycerides, stabilizing agents, antioxidants, glycerol, flavoring, colorant and odorant agents and other formulation components, used in the art of pharmaceutical and cosmetic formulary. A pharmaceutical or cosmetic composition manufactured according to the present invention is very easy to use. When applied onto the afflicted body surface of humans or animals, it is in a foam state, allowing free application without drip or spillage. Upon further application of a mechanical force, e.g., by rubbing the composition onto the body surface, it freely spreads on the surface and is rapidly absorbed.

Active Agents

It is to be understood that the active agents useful herein can in some instances provide more than one benefit or operate via more than one mode of action. Therefore, classifications herein are made for the sake of convenience and are not intended to limit the active agent to that particular application or applications listed.

The composition of the present invention comprises at least one active agent that provides therapeutic or cosmetic activity.

The composition of the present invention comprising at least one “active agent”, provides the following benefits: favorable spreadability and absorption, compared to conventional ointment, cream, lotion and the like; improved treatment convenience, leading to better compliance; enhanced delivery, leading to elevated bioavailability of the drug or cosmetic active agent in the target organ, thereby improving treatment efficacy.

In the context of the present invention, pharmaceutical and cosmetic active agents are included under the definition of at least one active agent. According to one embodiment the at least one active agent may be a single agent or a combination of agents that can be dissolved in the oleaginous carrier composition.

According to one embodiment, the at least one active agent is a hydrophobic agent, having solubility in distilled water at ambient temperature of less than about 1 gm per 100 mL, more preferable less than about 0.5 gm per 100 mL, and most preferably less than about 0.1 gm per 100 mL. In another embodiment, the at least one active agent is any therapeutic or cosmetic agent, providing that it is encapsulated in a hydrophobic envelope.

In another embodiment, the at least one active agent is insoluble and thus, incorporated in the foamable carrier of the present invention by suspension.

Non-limiting examples of active agents include antibiotics, antibacterials, antifungals, antivirals, steroidal antiinflammatory agents, steroids, non-steroidal anti-inflammatory agents, COX-1 inhibitors, COX-2 inhibitors, anesthetic agents, analgesics, antiallergic agents, corticosteroid, retinoids, calcineurin Inhibitors, nitric oxide synthase inhibitors, leucocyte chemotaxis inhibitors, dicarboxylic acids, vitamins, vitamin A and derivatives thereof, vitamin B and derivatives thereof, vitamin C and derivatives thereof, vitamin D and derivatives thereof, vitamin E and derivatives thereof, vitamin F and derivatives thereof, vitamin K and derivatives thereof, alpha hydroxy acids, beta hydroxy acids, keratolytic agents, calcium channel blockers, cholinergic drugs, nitric oxide donor, Immunosuppressant agents, immunoregulating agents, metal ion channel modulators, modulators of serotonin activity, serotonin reuptake inhibitors, antioxidants, cannabinoids, angiotensin II receptor antagonists, UDP-glucuronosyltransferase inhibitor, anti-neoplastic agents, vasoactive agents, lubricating agents and antiproliferative medications and mixtures thereof at any proportion. The concentration of said agents may be adopted to exert a therapeutic effect on a disease when applied to an afflicted area.

A general non-limiting list of hydrophobic active agents include abacavir, acebutolol, acrivastine, alatrofloxacin, albuterol, albendazole, alprazolam, alprenolol, amantadine, amiloride, aminoglutethimide, amiodarone, amitriptyline, amlodipine, amodiaquine, amoxapine, amphetamine, amphotericin, amprenavir, aminone, amsacrine, astemizole, atenolol, atropine, azathioprine, azelastine, azithromycin, baclofen, benethamine, benidipine, benzhexyl, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclocycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenyllmidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecamide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxaditine, roxithromycin, salbutamol, saquinavir, selegiline, sertraline, sibutramine, sildenafil, sparfloxacin, spiramycins, stavudine, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K5, vitamin K6, vitamin K7, zafirlukast, zolmitriptan, zolpidem, zopiclone, acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epalrestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenyloin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, levothyroxine, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenyloin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, vitamin K-S (II), zafirlukast, and pharmaceutically acceptable oil-soluble derivative and salts thereof.

Vasoactive Agents

Within the lists of active agents are included vasoactive agents such as minoxidil, sidenafil and caffeine.

In the context of the present invention, a vasoactive agent is a substance that changes the diameter of a blood vessel.

In one or more embodiments, the vasoactive agent is a vasodilator. A vasodilator is any of various agents that relax or widen blood vessels and thereby maintain or lower blood pressure.

Alteration in the release and action of endothelium-derived vasoactive factors is responsible for changes in vascular reactivity early in the course of vascular disease. These factors include nitric oxide, eicosanoids, endothelium-derived hyperpolarizing factor, endothelin, and angiotensin 11.

Nitric oxide (NO) has been recognized as an important messenger molecule having a broad spectrum of functions in many biological systems ranging from physiological control to pathological cytotoxic effect 1-3. Along with prostacyclin, NO is responsible for endothelium derived tonic relaxation of all types of blood vessels. NO is formed from L-arginine through the action of a family of isoenzymes, the nitric oxide synthases (NOS). Thus, in one or more embodiments, the vasoactive agent is selected from the group of therapeutic agents that modulate the production of nitric oxide or otherwise modulate or activate the effect of nitric oxide. In one or more embodiments, the vasoactive agent is selected from the group of therapeutic agents that modulate the activity of the enzyme nitric oxide synthase. In one or more embodiments, the vasoactive agent is selected from the group of therapeutic agents that enhance the effect of NO by inhibiting enzymes from the phosphodiesterase group, such as phosphodiesterase type 5 (PDE5).

In one or more embodiments, the vasoactive agent is selected from the group including nitrites, nitrates and their analogs, esters and salts. In one or more embodiments the vasoactive agent possesses a moiety selected from the group consisting of ONO, and ONO2.

Exemplary vasodilators include, but are not limited to, amyl nitrite, amyl nitrate, ethyl nitrite, butyl nitrite, isobutyl nitrite, glyceryl trinitrate, also known as nitroglycerin, octyl nitrite, sodium nitrite, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone (6,7-dimethoxycoumarin) and salts, isomers, analogs and derivatives thereof.

In one or more embodiments, the vasoactive agent belongs to a class of drugs that are known of possess vasodilator properties. Non limiting examples of drug classes that possess vasodilator properties include, but are not limited to, beta-adrenergic blockers, alpha-adrenoceptor blockers, prostaglandin and prostaglandin-like compounds, inhibitors of type 5 phosphodiesterase (PDE-5), angiotensin converting enzyme inhibitors, calcium antagonists, angiotensin II receptor antagonists, direct acting smooth muscle vasodilators, adrenergic inhibitors, endothelin antagonists, mineralocorticoid receptor antagonists, vasopeptidase inhibitors and renin inhibitors. Active agents belonging to such drug classes, as well as active agents belonging to other classes, which cause a vasodilator effect are also included in the scope of vasoactive agents according to the present invention.

Non-nitrate vasodilators from different classes include, but are not limited to sildenafil, dipyridamole, catecholamine, isoproternol, furosemide, prostaglandin, prostacyclin, enalaprilat (ACE-inhibitor), morphine (opiate), acepromazine (α-blocker), prazosin (α-blocker), enalapril (ACE-inhibitor), captopril (ACE-inhibitor), amlodipine (Ca channel blocker), minoxidil, tadalafil, vardenafil, phenylephrin, etilefein, caffeine, capsaicin and salts, isomers, analogs and derivatives thereof.

In one or more embodiments, the vasoactive agent is selected from the group of vasodilator peptides and proteins. Non-limiting examples of vasodilator paprides include, but are not limited to bradykinin, bradykinin-like peptide 1, bradykinin-like peptide III Phyllokinin (bradykinyl-isoleucyl-tyrosine O-sulfate), megascoliakinin ([Thr6]bradykinin-Lys-Ala), lysyl-bradykinin-like waspkinin, lysyl-bradykinin, maximakinin (Bombinakinin M), bombinakinin-GAP, kininogen-1 associated peptides, kininogen-2 associated peptides, T-kinin, thiostatin, prolixin-S, vespulakinin 2, vespakinin X, relaxin, adrenomedullin, ghrelin, maxadilan, substance P, calcitonin gene-related peptide (CGRP), Natriuretic peptides (NPs), e.g., atrial natriuretic peptide (ANP), C-type natriuretic peptide (CNP), and adrenomedullin (ADM), adrenomedullin, ovine corticotropin-releasing factor, sauvagine, urotensin and salts, isomers, analogs and derivatives thereof.

In one or more embodiments, the vasoactive agent is selected from the group of therapeutic agents that induce the production of a vasodilator peptide or otherwise enhance or activate the effect of a vasodilator peptide.

In one or more embodiments, the vasoactive agent is a substance derived or extracted from herbs having a vasodilator effect. Non limiting examples of herbs that contain vasoactive agents include achillea millefolium (Yarrow), allium sativum (garlic), amoracia rusticana (horseradish), berberis vulgaris (barberry), cimicifuga racemosa (black cohosh), coleus forskholii (coleus), coptis (Goldenthread), crataegus (hawthorn), eleutherococcus senticosus (siberian ginseng), ginkgo biloba(ginkgo), melissa offiicnalis (lemon balm), olea europaea (olive leaf), panax ginseng (Chinese ginseng), petroselinum crispum (parsley), scutellaria baicalensis (baical skullcap), tilia europaea (linden flower), trigonella foenumgraecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut, walnut and analogs and derivatives thereof.

According to one or more embodiments, the foamable composition includes a vasodilator and a vasoactive agent such that the vasodilator can have a synergistic effect by readily facilitating facile penetration of the vasoactive agent.

In one or more embodiments, the vasoactive agent is a vasoconstrictor. A vasoconstrictor is any of various agents that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness.

Other descriptive names of the vasoconstrictor group include vasoactive agonists, vasopressor agents and vasoconstrictor drugs. Certain vasoconstrictors act on specific receptors, such as vasopressin receptors or adrenoreceptors.

In one or more embodiments, the vasoconstrictor is a calcium channel agonist. Calcium channel agonists are agents that increase calcium influx into calcium channels of excitable tissues, thereby causing vasoconstriction.

Non limiting examples of vasoconstrictors include ephedrine, epinephrine, phenylephrine, angiotensin, vasopressin, and analogs and derivatives thereof.

In one or more embodiments, the vasoactive agent is a substance derived or extracted from herbs, having a vasoconstrictor effect.

Thus, in one or more embodiments, the vasoactive agent is a substance derived or extracted from a herbal source, selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom), cypress and salts, isomers, analogs and derivatives thereof.

Yet, in additional embodiments, the vasoactive agent is a metal oxide or a mineral, such as zinc oxide and bismuth subgallate.

The McKenzie vasoconstrictor assay, as described, for example, in the British Journal of Dermatology 1975; 93:563-71 and versions thereof, has been the primary method used for classifying the strength of a vasoconstrictor clinical efficacy. Thus, in one or more embodiments, the vasoactive agent is an agent that positively affects the vasoconstrictor assay.

Mixtures of these vasoactive agents may also be employed according to the present invention.

Anti-Infective Agents

Anti-infective agents include antibacterial, antifungal, antiviral, and anti-parasitic agents.

Antibacterial Agents

One important class of active agents comprises antibacterial agents. It is well known that bacterial infections are involved in a variety of superficial and non-superficial disorders of the skin and mucosal membranes. The antibacterial agent can be active against gram positive and gram-negative bacteria, protozoa, aerobic bacteria and anaerobes. The composition of the invention may include one or a combination of water soluble, oil soluble and suspended antibacterial agents.

Specific oil-soluble species of macrolide antibiotics, such as erythromycin; sulfonamide (in its base form), such as sulfanilamide, sulfadiazine and sulfacetamide; mupirocin; tetracyclines, such as tetracycline and doxycycline; specific oil-soluble species of synthetic and semi-synthesic penicillins and beta-lactams; cloramphenicol; specific oil-soluble species of imidazoles; dicarboxylic acids, such as azelaic acid; salicylates; peptide antibiotics; cyclic peptides, such as cyclosporine, tacrolimus, pimecrolimus and sirolimus (rapamycin); and non-specific antibacterial agents such as strong oxidants and free radical liberating compounds, bleaching agents, iodine compounds and benzoyl peroxide.

Antibacterial compositions according to the present invention may be used to treat infections of the skin. An example of a very common skin infection is impetigo, a bacterial disease caused by Staphylococcus aureus and beta-hemolytic streptococci, which mainly afflicts children and infants. Various antibacterial creams and ointments, such as mupirocin cream and mupirocin ointment, have been utilized to treat impetigo, however, treatment compliance is markedly impaired due to the fact that children resist the extensive rubbing involved in cream and ointment treatment. Foam, on the other hand, was found to be easily applied, without any difficulty. It has been surprisingly discovered that a composition of mupirocin n a vehicle containing PEG (as a potent solvent), a non-ionic surfactant and a gelling agent, where the non-ionic surface-active agent at a concentration of 2% by weight and the total amounts of surface-active agent is in the range of 2.5% by weight, and propellan, afforded an excellent foam which was stable upon discharge from the aerosol can and was easy to apply onto an afflicted area.

The composition of the present invention is particularly useful and beneficial in the prevention and treatment of secondary infections, accompanying skin-structure damage, such as in cuts, wounds, burns and ulcers. In all such cases, the present formulation is easy to use, being in foam state upon application and absorbing into the skin instantly upon gentle application.

While being useful in the prevention and treatment of infections, the antibacterial foam of the present invention is also applicable for decontaminating areas, afflicted with bacterial warfare organisms, such as anthrax and smallpox.

In the context of the present invention, an antibiotic agent is a substance, that has the capacity to inhibit the growth of or to destroy bacteria and other microorganisms.

In one or more embodiments, the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroides, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Beta-lactam antibiotics include, but are not limited to, 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, aziocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, ticarcillin and analogs, salts and derivatives thereof.

Aminoglycosides include, but are not limited to, 1,2′-N-DL-isoseryl-3′,4′-dideoxykanamycin B, 1,2′-N-DL-isoseryl-kanamycin B, 1,2′-N—[(S)-4-amino-2-hydroxybutyryl]-3′,4′-dideoxykanamycin B, 1,2′-N—[(S)-4-amino-2-hydroxybutyryl]-kanamycin B, 1-N-(2-Aminobutanesulfonyl) kanamycin A, 1-N-(2-aminoethanesulfonyl)3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl) kanamycin A, 1-N-(2-aminoethanesulfonyl) kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl) kanamycin A, 1-N-(2-aminopropanesulfonyl) kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B, 1-N-DL-isoserylkanamycin, 1-N-DL-isoserylkanamycin B, 1-N—[L-(−)-(alpha-hydroxy-gamma-aminobutyryl)]-XK-62-2,2′,3′-dideoxy-2′-fluorokanamycin A,2-hydroxygentamycin A3,2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin,3′,4′-dideoxyneamine,3′,4′-dideoxyribostamycin, 3′-deoxy-6′-N-methyl-kanamycin B, 3′-deoxyneamine,3′-deoxyribostamycin, 3′-oxysaccharocin,3,3′-nepotrehalosadiamine, 3-demethoxy-2″-N-formimidoylistamycin B disulfate tetrahydrate, 3-demethoxyistamycin B,3-O-demethyl-2-N-formimidoylistamycin B, 3-O-demethylistamycin B,3-trehalosamine,4″, 6″-dideoxydibekacin, 4-N-glycyl-KA-6606VI, 5″-Amino-3′,4′,5″-trideoxy-butirosin A, 6″-deoxydibekacin,6′-epifortimicin A, 6-deoxy-neomycin (structure 6-deoxy-neomycin B),6-deoxy-neomycin B, 6-deoxy-neomycin C, 6-deoxy-paromomycin, acmimycin, AHB-3′,4′-dideoxyribostamycin,AHB-3′-deoxykanamycin B, AHB-3′-deoxyneamine,AHB-3′-deoxyribostamycin,AHB-4″-6″-dideoxydibekacin, AHB-6″-deoxydibekacin,AHB-dideoxyneamine,AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gamma1, coumamidine gamma2,D,L-1-N-(alpha-hydroxy-beta-aminopropionyl)-XK-62-2, dactimicin,de-O-methyl-4-N-glycyl-KA-6606VI,de-O-methyl-KA-6606I, de-O-methyl-KA-7038I,destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, zygomycin and analogs, salts and derivatives thereof.

Ansa-type antibiotics include, but are not limited to, 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, tolypomycin and analogs, salts and derivatives thereof.

Antibiotic anthraquinones include, but are not limited to, auramycin, cinerubin, ditrisarubicin, ditrisarubicin C, figaroic acid fragilomycin, minomycin, rabelomycin, rudolfomycin, sulfurmycin and analogs, salts and derivatives thereof.

Antibiotic azoles include, but are not limited to, azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, voriconazol and analogs, salts and derivatives thereof.

Antibiotic glycopeptides include, but are not limited to, acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, zorbamycin and analogs, salts and derivatives thereof.

Macrolides include, but are not limited to, acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, venturicidin and analogs, salts and derivatives thereof.

Antibiotic nucleosides include, but are not limited to, amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, vengicide and analogs, salts and derivatives thereof.

Antibiotic peptides include, but are not limited to, actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, berninamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, melittin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, zervacin and analogs, salts and derivatives thereof.

In one or more embodiments, the antibiotic peptide is a naturally-occurring peptide that possesses an antibacterial and/or an antifungal activity. Such peptide can be obtained from a herbal or a vertebrate source.

Polyenes include, but are not limited to, amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, trichomycin and analogs, salts and derivatives thereof.

Polyethers include, but are not limited to, 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, salinomycin and analogs, salts and derivatives thereof.

Quinolones include, but are not limited to, an alkyl-methylendioxy-4(1H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin and analogs, salts and derivatives thereof.

Antibiotic steroids include, but are not limited to, aminosterol, ascosteroside, cladosporide A, dihydrofusidic acid, dehydro-dihydrofusidic acid, dehydrofusidic acid, fusidic acid, squalamine and analogs, salts and derivatives thereof.

Sulfonamides include, but are not limited to, chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfacarbamide and analogs, salts and derivatives thereof.

Tetracyclines include, but are not limited to, dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, tetracycline and analogs, salts and derivatives thereof. Tetracyclines are very sensitive to water and therefore, there inclusion in a formulation that contains no water is beneficial to their shelf life stability.

Dicarboxylic acids, having between about 6 and about 14 carbon atoms in their carbon atom skeleton are particularly useful in the treatment of disorders of the skin and mucosal membranes that involve microbial. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments of the present invention, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred. It has antibacterial effects on both aerobic and anaerobic organisms, particularly propionibacterium acnes and staphylococcus epidermidis, normalizes keratinization, and has a cytotoxic effect on malignant or hyperactive melanocytes. In a preferred embodiment, the dicarboxylic acid is azelaic acid in a concentration greater than 10%. Preferably, the concentration of azelaic acid is between about 10% and about 25%. In such concentrates, azelaic acid is suitable for the treatment of a variety of skin disorders, such as acne, rosacea and hyperpigmentation.

In one or more embodiments, the antibiotic agent is an antibiotic metal. A number of metals ions been shown to possess antibiotic activity, including silver, copper, zinc, mercury, tin, lead, bismutin, cadmium, chromium and ions thereof. It has been theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the antibiotic metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The antibiotic metal can further be intercalated in a chelating substrate.

In further embodiments, the antibiotic metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

In one or more embodiments, the antibiotic metal or metal ion is embedded into a substrate, such as a polymer, a mineral (such as zeolite, clay and silica).

Oxidizing agents and substances that release free radicals and/or active oxygen. In one or more embodiments, the antibiotic agent comprises strong oxidants and free radical liberating compounds, such as oxygen, hydrogen peroxide, benzoyl peroxide, elemental halogen species, as well as oxygenated halogen species, bleaching agents (e.g., sodium, calcium or magnesium hypochloride and the like), perchlorite species, iodine, iodate, and benzoyl peroxide. Organic oxidizing agents are also included in the definition of “oxidizing agent” according to the present invention, such as quinones. Such agents possess a potent broad spectrum activity

In one or more embodiments the antibiotic agent is a cationic antimicrobial agent. The outermost surface of bacterial cells universally carries a net negative charge, making them sensitive to cationic substances. Examples of cationic antibiotic agents include: quaternary ammonium compounds (QAC's)—QAC's are surfactants, generally containing one quaternary nitrogen associated with at least one major hydrophobic moiety; alkyltrimethyl ammonium bromides are mixtures of where the alkyl group is between 8 and 18 carbons long, such as cetrimide (tetradecyltrimethylammonium bromide); benzalkonium chloride, which is a mixture of n-alkyldimethylbenzyl ammonium chloride where the alkyl groups (the hydrophobic moiety) can be of variable length; dialkylmethyl ammonium halides; dialkylbenzyl ammonium halides; and QAC dimmers, which bear bi-polar positive charges in conjunction with interstitial hydrophobic regions.

In one or more embodiments, the antibiotic agent is selected from the group of biguanides, triguanides, bisbiguanides and analogs thereof.

Guanides, biguanides, biguanidines and triguanides are unsaturated nitrogen containing molecules that readily obtain one or more positive charges, which make them effective antimicrobial agents. The basic structures a guanide, a biguanide, a biguanidine and a triguanide are provided below.

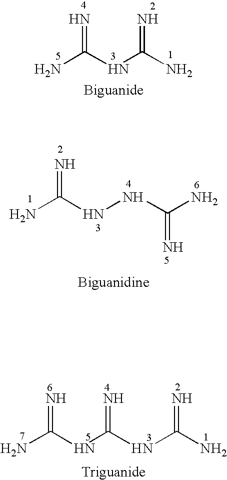

In one or more preferred embodiments, the guanide, biguanide, biguanidine or triguanide, provide bi-polar configurations of cationic and hydrophobic domains within a single molecule.

Examples of guanides, biguanides, biguanidines and triguanides that are currently been used as antibacterial agents include chlorhexidine and chlorohexidine salts, analogs and derivatives, such as chlorhexidine acetate, chlorhexidine gluconate and chlorhexidine hydrochloride, picloxydine, alexidine and polihexanide. Other examples of guanides, biguanides, biguanidines and triguanides that can conceivably be used according to the present invention are chlorproguanil hydrochloride, proguanil hydrochloride (currently used as antimalarial agents), metformin hydrochloride, phenformin and buformin hydrochloride (currently used as antidiabetic agents).

In one or more embodiments, the cationic antimicrobial agent is a polymer.

Cationic antimicrobial polymers include, for example, guanide polymers, biguanide polymers, or polymers having side chains containing biguanide moieties or other cationic functional groups, such as benzalkonium groups or quarternium groups (e.g., quaternary amine groups). It is understood that the term “polymer” as used herein includes any organic material comprising three or more repeating units, and includes oligomers, polymers, copolymers, block copolymers, terpolymers, etc. The polymer backbone may be, for example a polyethylene, ploypropylene or polysilane polymer.

In one or more embodiments, the cationic antimicrobial polymer is a polymeric biguanide compound. When applied to a substrate, such a polymer is known to form a barrier film that can engage and disrupt a microorganism. An exemplary polymeric biguanide compound is polyhexamethylene biguanide (PHMB) salts. Other exemplary biguanide polymers include, but are not limited to poly(hexamethylenebiguanide), poly(hexamethylenebiguanide) hydrochloride, poly(hexamethylenebiguanide) gluconate, poly(hexamethylenebiguanide) stearate, or a derivative thereof. In one or more embodiments, the antimicrobial material is substantially water-insoluble.

Yet, in one or more embodiment, the antibiotic is a non-classified antibiotic agent, including, without limitation, aabomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis immune substance, anti-tuberculosis immune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′,5′-cyclohexadienyl) derivative, lanomycin, leinamycin, leptomycin, libanomycin, lincomycin, lomofungin, lysolipin, magnesidin, manumycin, melanomycin, methoxycarbonylmethylmonate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, prop-2-enylmonate, protomycin, pseudomonas antibiotic, pseudomonic acid, purpuromycin, pyrinodemin, pyrroInitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs, salts and derivatives thereof.

In one or more embodiments, the antibiotic agent is a naturally occurring antibiotic compound. As used herein, the term “naturally-occurring antibiotic agent” includes all antibiotic that are obtained, derived or extracted from plant or vertebrate sources. Non-limiting examples of families of naturally-occurring antibiotic agents include phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine and novoimanine.

Ciclopirox and ciclopiroxolamine possess fungicidal, fungistatic and sporicidal activity. They are active against a broad spectrum of dermatophytes, yeasts, moulds and other fungi, such as trichophyton species, microsporum species, epidermophyton species and yeasts (candida albicans, candida glabrata, other candida species and cryptococcus neoformans). Some aspergillus species are sensitive to ciclopirox as are some penicillium. Likewise, ciclopirox is effective against many gram-positive and gram-negative bacteria (e.g., escherichia coli, proteus mirabilis, pseudomonas aeruginosa, staphylococcus and streptococcus species), as well as mycoplasma species, trichomonas vaginalis and actinomyces.

Plant oils and extracts which contain antibiotic agents are also useful. Non limiting examples of plants that contain agents include thyme, perilla, lavender, tea tree, terfezia clayeryi, Micromonospora, putterlickia verrucosa, putterlickia pyracantha putterlickia retrospinosa, Maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover and yellow dock, garlic and St. John's wort.

Mixtures of these antibiotic agents may also be employed according to the present invention.

Anti-fungal agents

Fungal infections are another object of treatment using the composition of the present invention. Superficial fungal infection of the skin is one of the most common skin diseases seen in general practice. Dermatophytosis is probably the most common superficial fungal infection of the skin. It is caused by a group of fungi, which are capable of metabolizing the keratin of human epidermis, nails or hair. There are three genera of dermatophytes causing dermatophytosis, i.e, microsporum, trichophyton and epidermophyton.

Candidiasis is an infection caused by the yeast like fungus candida albicans or occasionally other species of candida. Clinical syndromes of candidiasis include: (a) oral candidiasis (thrush); (b) candidiasis of the skin and genital mucous membrane; and (c) candida paronychia, which inflicts the nail and nail bed.

The antifungal agent, also termed “antimycotic”. The terms “antifungal” and “antimycotic” as used herein include, but is not limited to, any substance being destructive to or inhibiting the growth of fungi and yeast or any substance having the capacity to inhibit the growth of or to destroy fungi and/or yeast.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina. Thus, in one or more embodiments, the antifungal agent is selected from the group including but not limited to, azoles, diazoles, triazoles, miconazole, fluconazole, ketoconazole, clotrimazole, itraconazole, Climbazole, griseofulvin, ciclopirox, ciclopirox-olamine, amorolfine, terbinafine, Amphotericin B, potassium iodide and flucytosine (5FC) at a therapeutically effective concentration.

The foam composition of the present invention may comprise an antifungal drug, which is active against dermatophytes and candida, selected from the group of, but not limited to azoles, diazoles, triazoles, miconazole, fluconazole, ketoconazole, clotrimazole, itraconazole griseofulvin, ciclopirox, amorolfine, terbinafine, Amphotericin B, potassium iodide, flucytosine (5FC) and any combination thereof at a therapeutically effective concentration.

The composition of the present invention is useful for example for the treatment and prevention of tinea corporis, tinea pedis, tinea rubrum, tinea unguium, tinea cruris, tinea barbae and tinea versicolor, as well as yeast infections, such as candidiasis, and candidal vaginitis.

Azoles are pharmaceutically active compounds that are unsaturated five member ring heterocyclic compound, wherein one, two or three members of the ring are nitrogen atoms, as exemplified in a non-limiting way and illustrated in the following schemes:

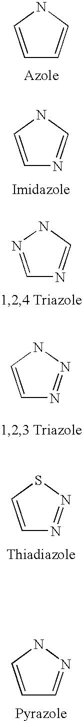

The azole is a compound including an unsaturated five member ring heterocyclic compound, wherein one, two or three members of the ring are nitrogen atoms.

Examples of therapeutic azoles include, but are not limited to, azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol and salts and derivatives thereof. Such azoles are mainly used as antifungal agents, yet several of them also possess other therapeutic benefits, such as anti-inflammatory, antibacterial and antiviral effects.

Additional non-limiting exemplary classes of azoles include oxazoles, thiazoles, thiadiazoles and thiatriazoles, benzimidazoles, and salts and derivatives thereof.

In an embodiment of the present invention, the azole is metronidazole.

In one or more embodiments, the antifungal agent is a peptide. In certain embodiments, antifungal agent is a naturally-occurring peptide that possesses an antibacterial and/or an antifungal activity. Such peptide can be obtained from a herbal or a vertebrate source.

In an embodiment of the present invention, the antifungal agent is a polyene. Polyene compounds are so named because of the alternating conjugated double bonds that constitute a part of their macrolide ring structure. Polyenes include, but are not limited to, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, oxypricin, partricin, pentamycin, perimycin, pimaricin, primycin, proticin, rimocidin, sistomycosin, sorangicin, trichomycin and analogs, salts and derivatives thereof.

In an embodiment of the present invention, the antifungal agent is a pyrimidine, such as Flucytosine.

In an embodiment of the present invention, the antifungal agent is an allylamine, such as terbinafine and naftifine.

In an embodiment of the present invention, the antifungal agent is a morpholine derivative, such as amorolfine.

In an embodiment of the present invention, the antifungal agent is selected from the group consisting of Ciclopirox, ciclopiroxolmine, griseofulvin,

In an embodiment of the present invention, the antifungal agent is a Thiocarbamate, such as tolnaftate.

In an embodiment of the present invention, the antifungal agent is a Sulfonamide, such as Mafenide and Dapsone.

In an embodiment of the present invention, the antifungal agent consists of a plant oil or a plant extract possessing antifungal activity; or a plant oil or extract which contains antifungal agents. Non-limiting examples of plants containing agents include, but are not limited to, anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, garlic, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.

In an embodiment of the present invention, the antifungal agent is an anti-microbial metal. A number of metals ions been shown to possess antibiotic activity, including silver, copper, zinc, mercury, tin, lead, bismutin, cadmium, chromium and ions thereof. It has been theorized that these anti-microbial metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the anti-microbial metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The anti-microbial metal can further be intercalated in a chelating substrate.

In further embodiments, the anti-microbial metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

Yet, in another embodiment, the antifungal agent is an oxidizing agent or a substance that releases free radicals and/or active oxygen. Exemplary oxidizing agents are hydrogen peroxide, benzoyl peroxide, elemental halogen species (compounds), as well as oxygenated halogen species (compounds), bleaching agents (e.g., sodium, calcium or magnesium hypochloride and the like), perchlorite species (compounds), iodine and iodate compounds. Organic oxidizing agents are also included in the definition of “oxidizing agent” according to the present invention, such as quinones. Such agents possess a potent broad spectrum activity

In further embodiments the antifungal agent is a cationic antimicrobial agent. The outermost surface of bacterial and fungal cells universally carries a net negative charge, making them sensitive to cationic substances. Examples of cationic antibiotic agents include: quaternary ammonium compounds, such as alkyltrimethyl ammonium bromides, benzalkonium chloride, dialkylbenzyl ammonium halides, and dimmers thereof, which bear bi-polar positive charges in conjunction with interstitial hydrophobic regions.

Anti-Viral Agents

The composition of the present invention is particularly beneficial in treating and preventing viral infections. Cold sores are caused by the herpes simplex Type 1 virus and are sometimes referred to as facial herpes. Mollusca are small viral growths that appear singly or in groups on the face, trunk, lower abdomen, pelvis, inner thighs, or penis. Shingles (herpes zoster), which usually only occurs once in a lifetime, appears as a rash (clusters of blisters with a red base). It is caused by the same virus responsible for chickenpox. Warts are a common, benign skin tumor caused by viral infection.

Any known antiviral agent, in a therapeutically effective concentration, can be incorporated in the foam composition of the present invention. The composition of the present invention, which comprises a hydrophobic solvent, would facilitate an enhanced rate of penetration and better topical distribution of any of the above listed antiviral drugs.

Steroids

In the context of the present invention, steroids are compounds possessing the skeleton of cyclopenta[a]phenanthrene or a skeleton derived therefrom by one or more bond scissions or ring expansions or contractions. Methyl groups are normally present at C-10 and C-13. An alkyl side chain may also be present at C-17. Sterols are steroids carrying a hydroxyl group at C-3 and most of the skeleton of cholestane. Additional carbon atoms may be present in the side chain.

Steroids are numbered and rings are lettered as in formula 1. If one of the two methyl groups attached to C-25 is substituted it is assigned the lower number (26); if both are substituted, that carrying the substituent cited first in the alphabetical order is assigned the lower number.

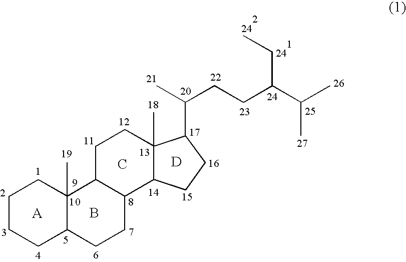

The steroids can have substituents on the steroid side chain as exemplified in formula 4-7:

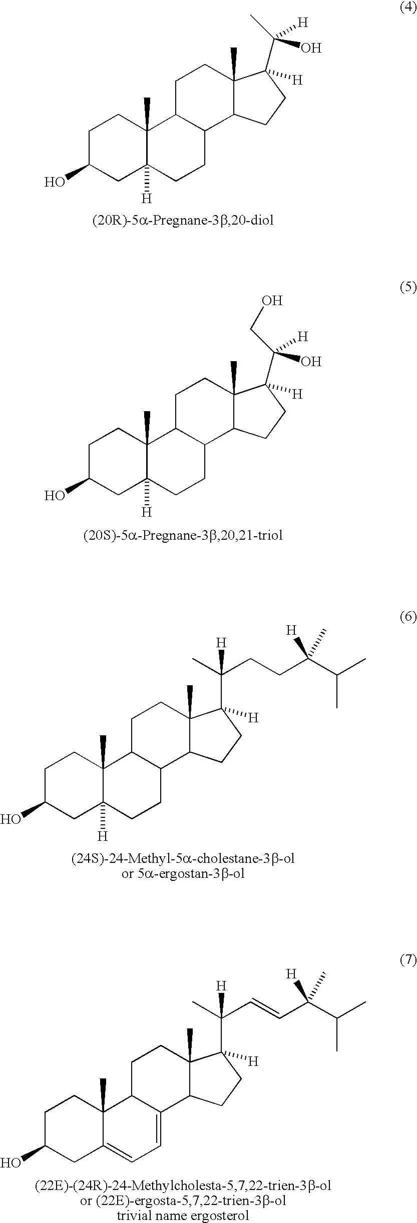

The steroids can have the formalae as exemplified in formula 9-18. In one or more embodiments, the steroid or sterol has no substitution at C-17, as exemplified by gonane, e.g., formulae 9 and 10, estrange (also termed oestrane), e.g. formulae II and 12, and androstane, e.g., formulae 13 and 14. In one or more embodiments, the steroid or sterol has methyl groups at both C-10 and C-13 and a side chain R at C-17 (formulae 15 and 16), as exemplified in Table 1.

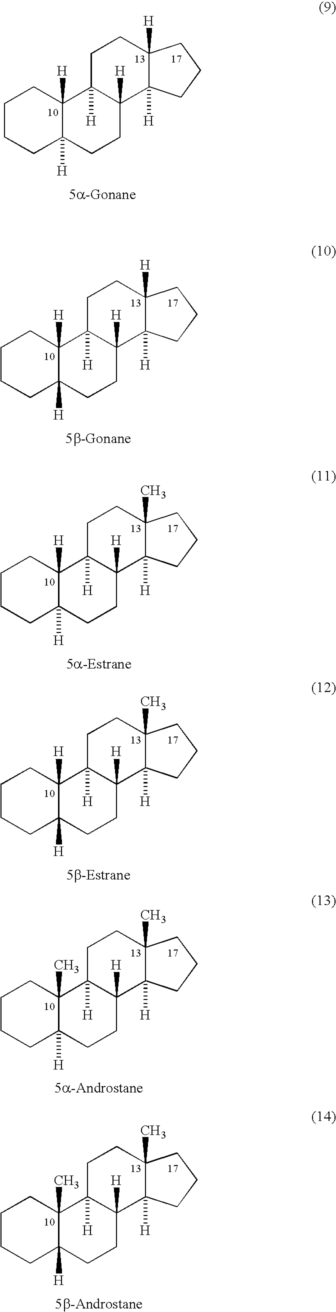

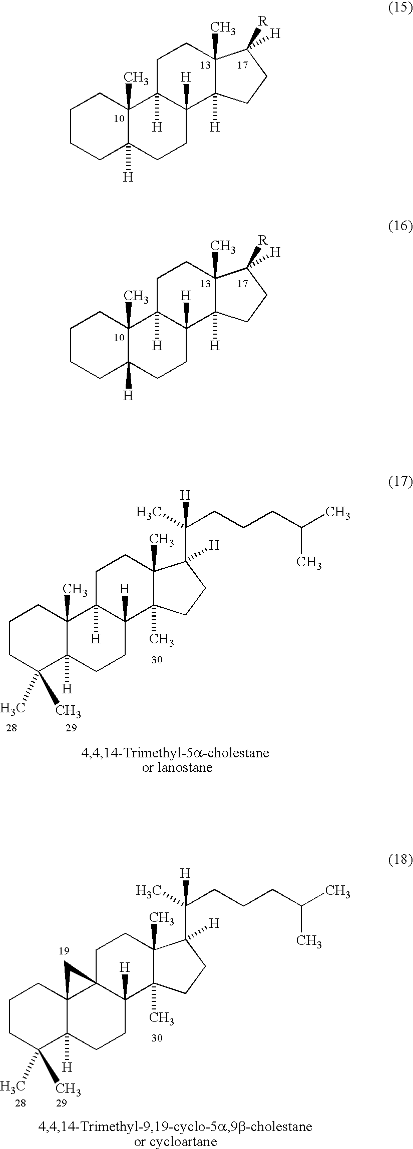

TABLE 1
Hydrocarbons with side chain at C-17

| Side chain | 5α-Series (15) | 5^{β}-Series (16) |
|---|---|---|
| 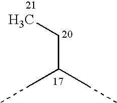 | 5α-pregnane (allopregnane) | 5^{β}-pregnane |
| 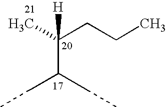 | 5α-cholane (allocholane) | 5^{β}-cholane |
| 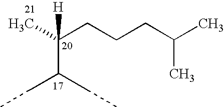 | 5α-cholestane | 5^{β}-cholestane (coprostane) |
| 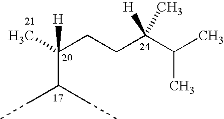 | 5α-ergostane | 5^{β}-ergostane |
| 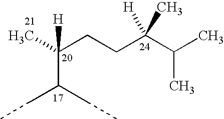 | 5α-campestane | 5^{β}-campestane |
| 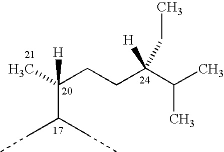 | 5α-poriferastane | 5^{β}- poriferastane |
| 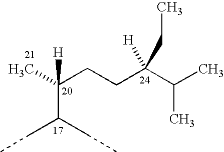 | 5α-stigmastane | 5^{β}-stigmastane |
| 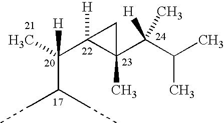 | 5α-gorgostane | 5^{β}-gorgostane |

Examples of unsaturated steroids and sterols are provided in formulae 19-22:

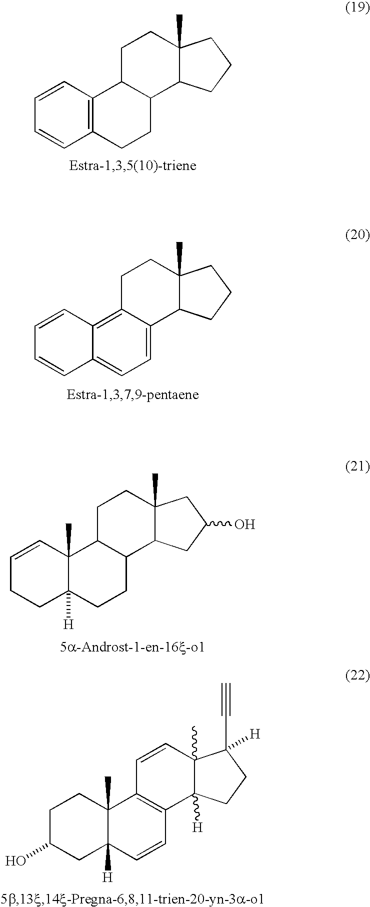

The stereochemistry of double bonds in the side chain is indicated using the E,Z convention. The same applies to the seco compounds of the vitamin D series (example in formula 23). In certain cases, the steroid has two carbon chains attached at position 17, e.g. 17-methyl-5α-pregnane 24, 17-methyl-5α, 17β-pregnane 25, and 17-ethyl-5-cholestane and 17-(2-bromoethyl)-5α, 17α-cholestane 26. Other examples of a steroid that has two carbon chains attached at position 17, are 17,17-dimethyl-5α-androstane 27 and 17β-methyl-17α-propyl-5α-androstane 28. In certain embodiments, the carbon skeleton of a steroid a carbon atom is replaced by a hetero atom, as exemplified by 17β-hydroxy-4-oxaandrost-5-en-3-one 29. Yet, in additional embodiments, an additional ring is formed by means of a direct link between any two carbon atoms of the steroid ring system or the attached side chain, as exemplified by formulae 30, 31 and 32.

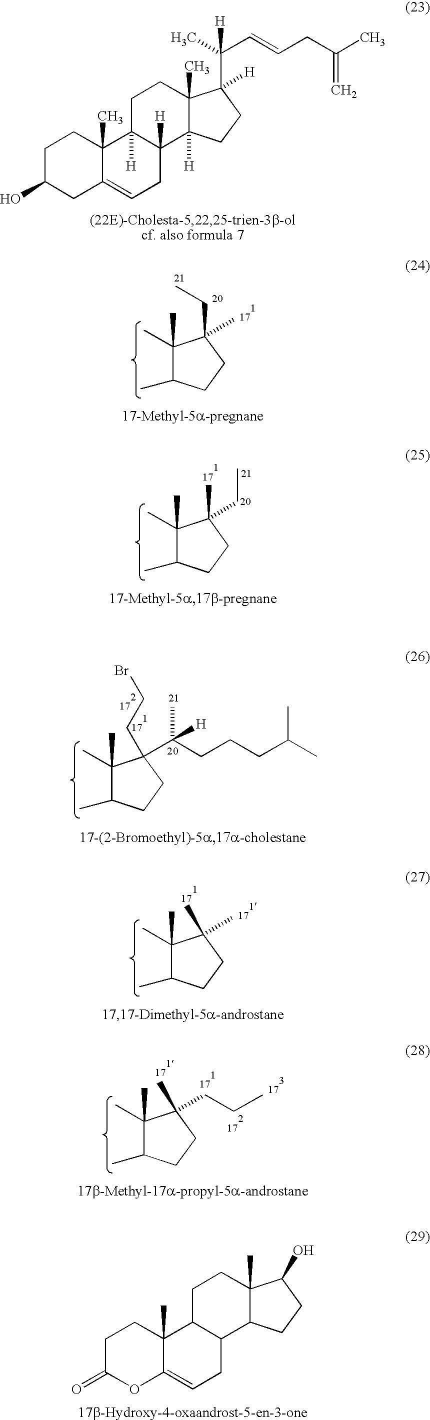

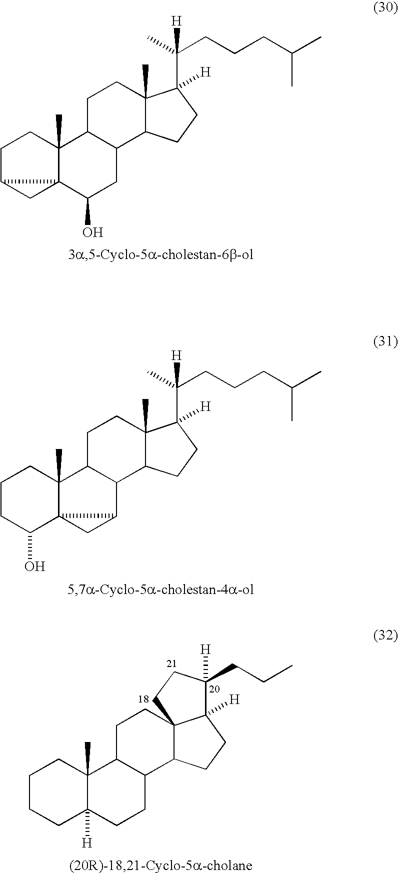

Many important naturally occurring steroids contain one or more additional heterocyclic ring(s), fused or attached to ring D, formed by modifications of the side chain. These steroids can be grouped into the following families: (a) cardanolides, e.g., 5β-cardanolide 33, 3β,14-dihydroxy-5β-card-20(22)-enolide (digitoxigenin) 34 and 3β,5,14-trihydroxy-19-oxo-5β,-card-20(22)-enolide (strophanthidin) 35, as well as epoxycardanolides, containing a 14,21- or a 16,21-oxygen bridge, as shown in 36, (b) bufanolides, e.g., structures 37-39, (c) spirostans, e.g., structures 40-43, (d) furostans, e.g., structures 44-45, and (e) steroid alkaloids.

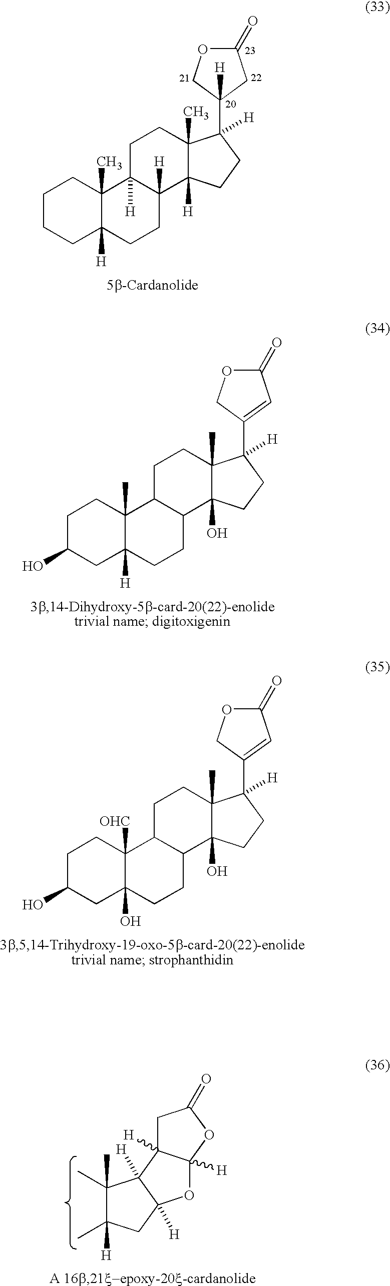

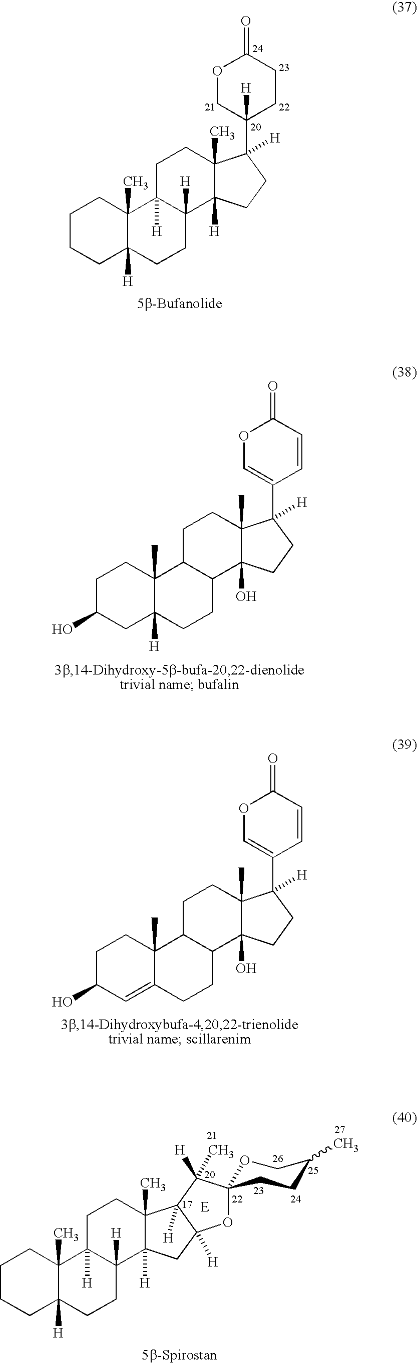

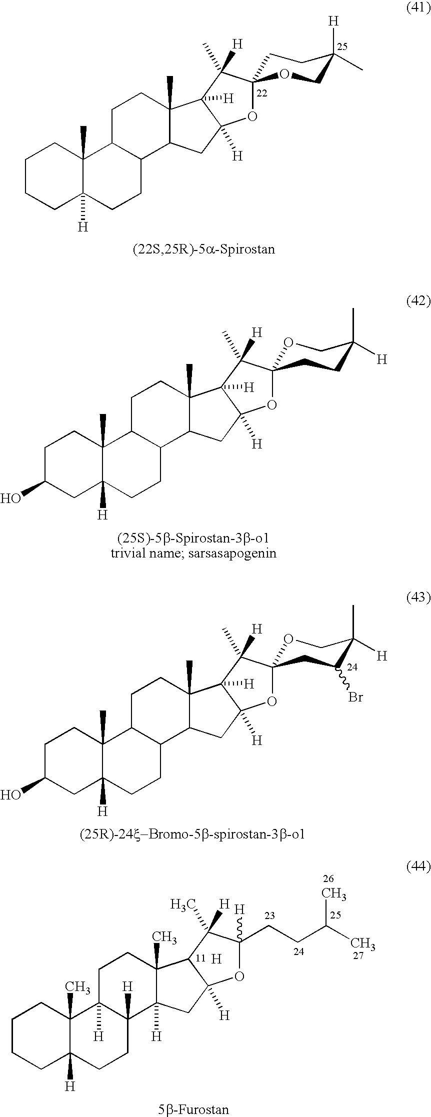

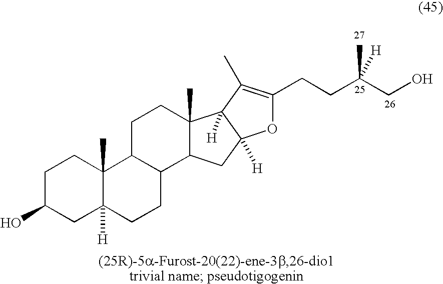

Several biologically important steroids are derivatives of the parent hydrocarbons carrying various functional groups. Some of the common functional groups include but are not limited to halogens, alkyl groups, aryl groups, benzyl groups, carboxy groups and alkoxy groups.

In one or more embodiments, the steroid is selected from the group consisting of an acid, a salt of an acid, as exemplified in formulae 46-49, and esters, as exemplified in formulae 50 and 51. In one or more embodiments, the steroid is a lactone, as exemplified in formulae 52-54.

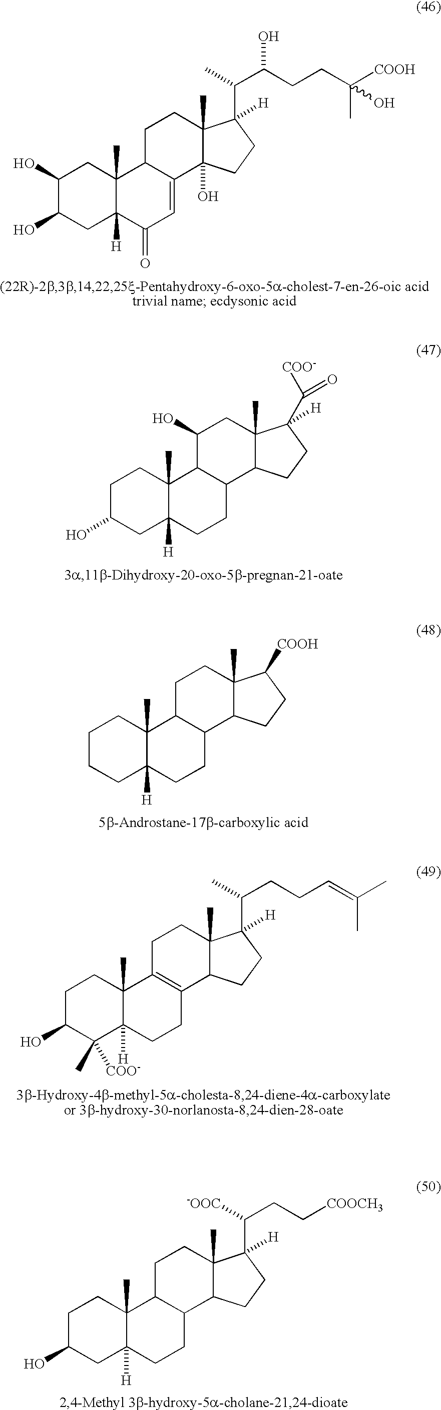

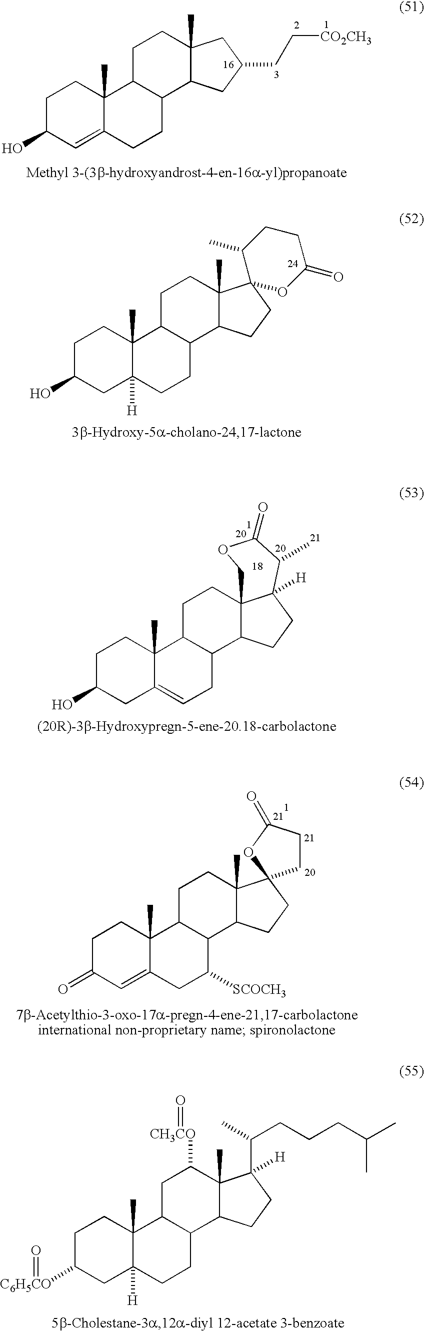

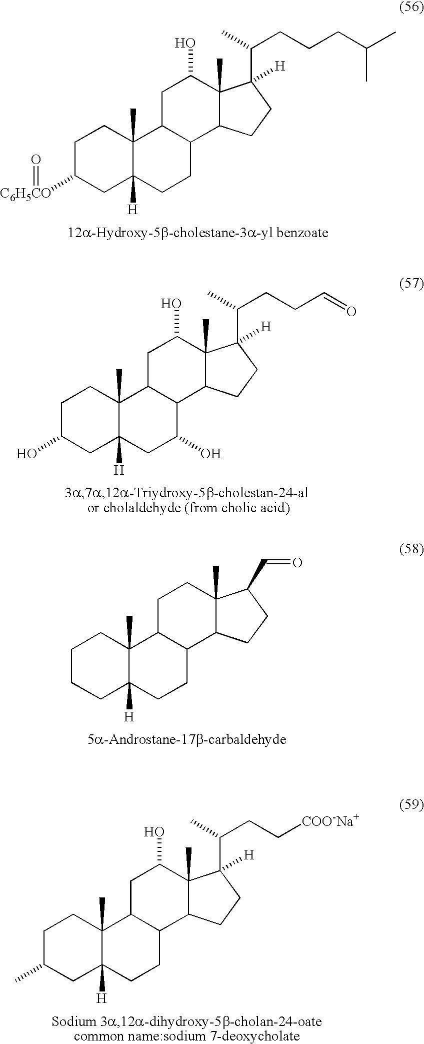

In one or more embodiments, the steroid is an ester of a steroid alcohol, as exemplified by 5-cholestan-3-yl acetate, 5-cholestane-3,12-diyl diacetate, 3-oxoandrost-4-en-17-yl acetate (trivial name testosterone acetate), 17-hydroxy-20-oxopregn-5-en-3-yl sulfate, 3-acetoxy-5-cardanolide, 3-benzoyloxy-11-hydroxy-20-oxo-5-pregnan-21-oate (monobenzoate of 47), 3-acetoxy-5-cholano-24,17-lactone (acetate of 52), 3-O-acetylcholic acid, 17-O-benzoylestradiol-17, 3-O-linolenoylcholesterol, as well as in formulae 55 and 56.

In one or more embodiments, the steroid is an oxo compound. The oxo compound can be an aldehde, as exemplified by 5-androstan-19-al, 5-cholan-24-al, 3-formyl-5-cholan-24-oic acid and by formulae 57 and 58, or a ketone, as exemplified by 5-androstan-3-one, pregn-5-ene-3,20-dione and 11-oxo-5-cholan-24-oic acid.

In one or more embodiment, the steroid is an alcohol as exemplified by 5-cholestane-3,11-diol, 3-hydroxy-5-androstan-17-one (trivial name: androsterone) and by formulae 59.

In additional embodiments, the steroid is an amine as exemplified by androst-5-en-3-amine and formula 60, an ether as exemplified by 17-methoxyandrost-4-en-3-one, (20S)-3,17,20-trimethoxy-5-pregnane, (20S)-3,17-dimethoxy-5-pregnan-20-ol, 21-O-methylcortisol and formula 61, an acetal or a ketal of an oxo steroid (also named as dialkoxy steroids) as exemplified by 3,3-dimethoxycholest-4-ene, 2,3-(methylenedioxy)pregn-5-ene and formula 62.

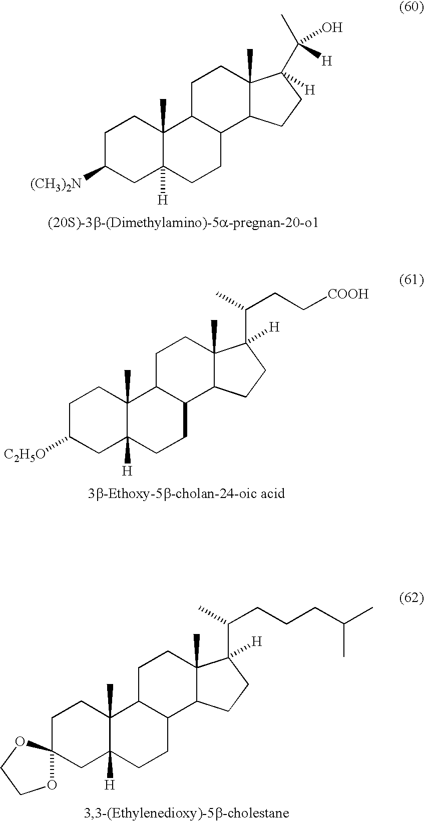

Examples of trivial names retained for important steroid derivatives, these being mostly natural compounds of significant biological activity, are given in Table 2.

TABLE 2
Trivial names of some important steroid derivatives

| Trivial name | Systematic steroid name |
|---|---|
| Aldosterone | 18,11-hemiacetal of 11^{β},21-dihydroxy-3,20-dioxopregn-4-en- |
| | 18-al or 11^{β},18-epoxy-18^{ξ},21-dihydroxypregn-4-ene-3,20- |
| | dione |
| Androsterone | 3α-hydroxy-5α-androstan-17-one |
| Brassinolide | (22R,23R)-2α,3α,22,23-tetrahydroxy-6,7-seco-5α- |
| | cmpestano-6,7-lactone |
| Calcidiol (93) | (5Z,7E)-(3S)-9,10-secocholesta-5,7,10(19)-triene-3,25-diol |
| Calciol = cholecalciferol (92) | (5Z,7E)-(3S)-9,10-secocholesta-5,7,10(19)-trien-3-ol |
| Calcitriol (94) | (5Z,7E)-(1S,3R)-9,10-secocholesta-5,7,10(19)-triene-1,3,25- |
| | triol |
| Cholesterol | cholest-5-en-3^{β}-ol |
| Cholic acid | 3α,7α,12α-trihydroxy-5^{β}-cholan-24-oic acid |
| Corticosterone | 11^{β},21-dihydroxypregn-4-ene-3,20-dione |
| Cortisol | 11^{β},17,21-trihydroxypregn-4-ene-3,20-dione |
| Cortisol acetate | 21-O-acetylcortisol |
| Cortisone | 17,21-dihydroxypregn-4-ene-3,11,20-trione |
| Cortisone acetate | 21-O-acetylcortisone |
| Deoxycorticosterone | 21-hydroxypregn-4-ene-3,20-dione (i.e. the 11-deoxy |
| | derivative of corticosterone) |
| Ecdysone | (22R)-2^{β},3^{β},14α,22,25-pentahydroxy-5^{β}-cholest-7-en-6-one |
| Ercalciol = ergocalciferol | (5Z,7E,22E)-(3S)-9,10-secoergosta-5,7,10(19),22-tetren-3-ol |
| Ergosterol (7) | (22E)-ergosta-5,7,22-trien-3^{β}-ol |
| Estradiol-17α | estra-1,3,5(10)-triene-3,17α-diol |
| Estradiol-17^{β} | estra-1,3,5(10)-triene-3,17^{β}-diol |
| Estriol | estra-1,3,5(10)-triene-3,16α,17^{β}-triol |
| Estrone | 3-hydroxyestra-1,3,5(10)-trien-17-one |
| Lanosterol | lanosta-8,24-dien-3^{β}-ol |
| Lithocholic acid | 3α-hydroxy-5^{β}-cholan-24-oic acid |
| Progesterone | pregn-4-ene-3,20-dione |
| Pseudotigogenin | (25R)-5α-furost-20(22)-ene-3^{β},26-diol |
| Sarsasapogenin | (25S)-5^{β}-spirostan-3^{β}-ol |
| Smilagenin | (25R)-5^{β}-spirostan-3^{β}-ol |
| Testosterone (63) | 17^{β}-hydroxyandrost-4-en-3-one |
| Tigogenin | (25R)-5α-spirostan-3^{β}-ol |

Additional non-limiting examples of steroids that are applicable according to the present invention are provided in formulae 63-79.

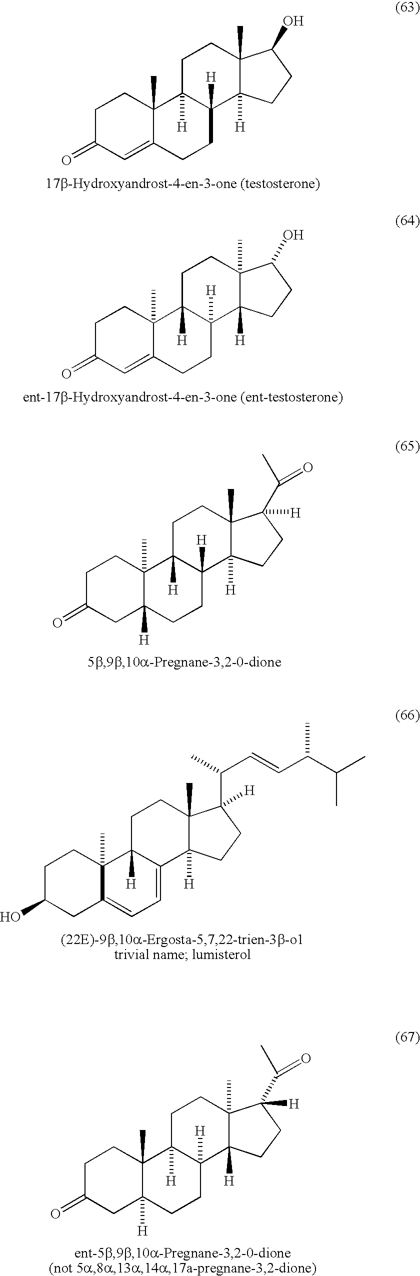

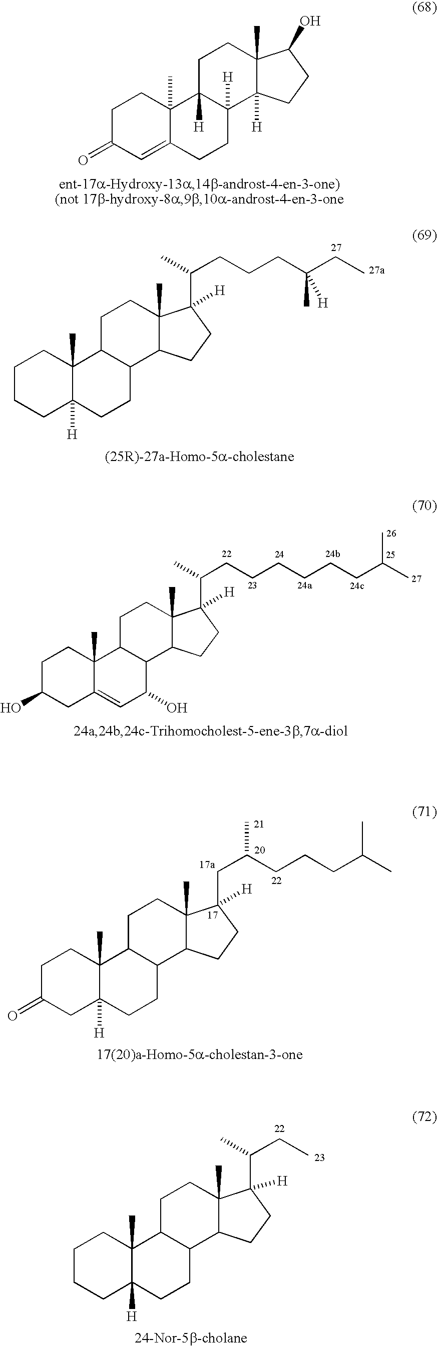

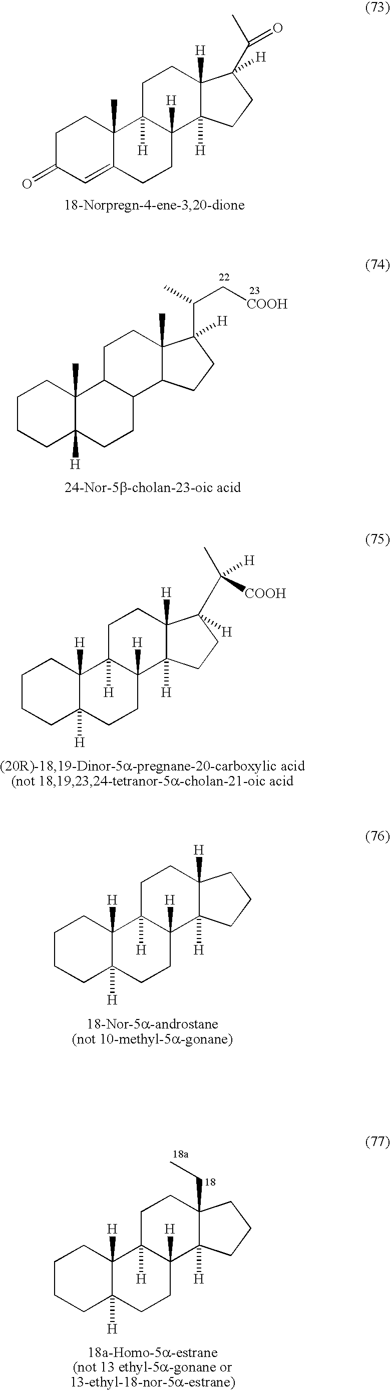

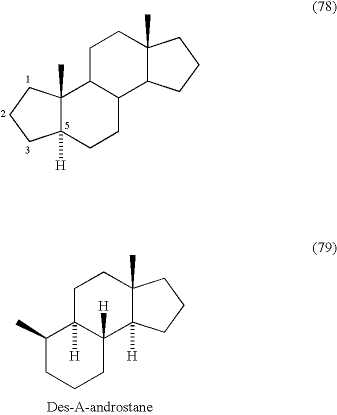

In one or more embodiments according to the present invention, the steroid is a compound, in which one or more of the cyclopenta[a]phenanthrene rings is contracted by loss of an unsubstituted methylene group, as exemplified by 4-nor-5-androstane (78), where C-4 is missing. In other embodiments one or more of the cyclopenta[a]phenanthrene rings is expanded by inclusion of a methylene group, as exemplified by formulae 80-86.

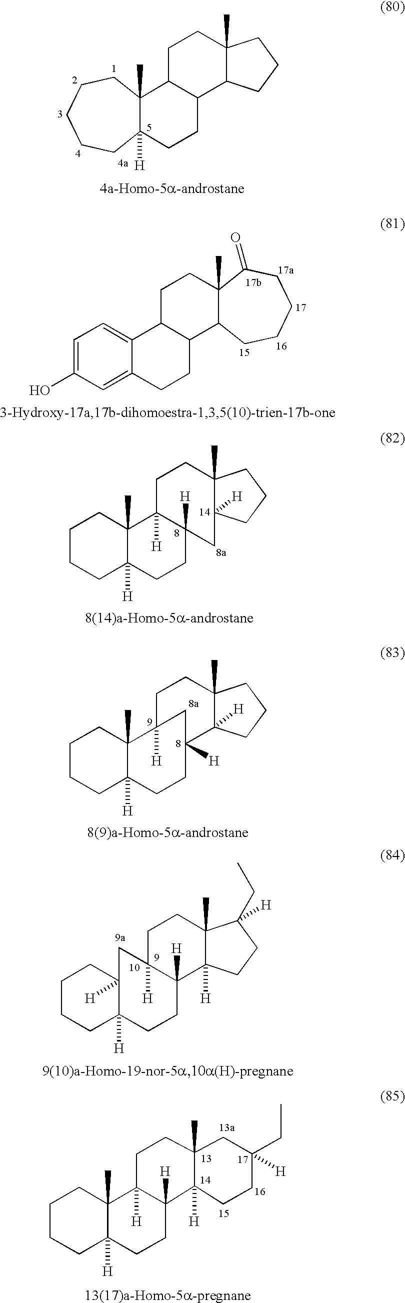

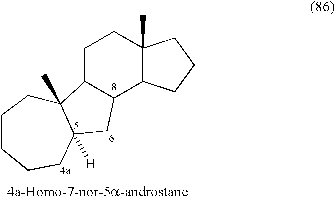

In one or more embodiments, the steroid contains additional rings that are formed within, or on, a steroid nucleus. In additional embodiments, the steroids contains a bivalent bridge such as —O—O—, —[CH₂]ₙ—, linking non-adjacent ring positions as exemplified by formulae 99-102.

In one or more embodiments, the steroid contains a cyclopenta[a]phenanthrene skeleton and a carbocyclic or heterocyclic ring component fused to it, as exemplified by formulae 103-111, and in other embodiments, an additional ring is linked to the cyclopenta[a]phenanthrene skeleton through a spiro system, as exemplified by formula 112.

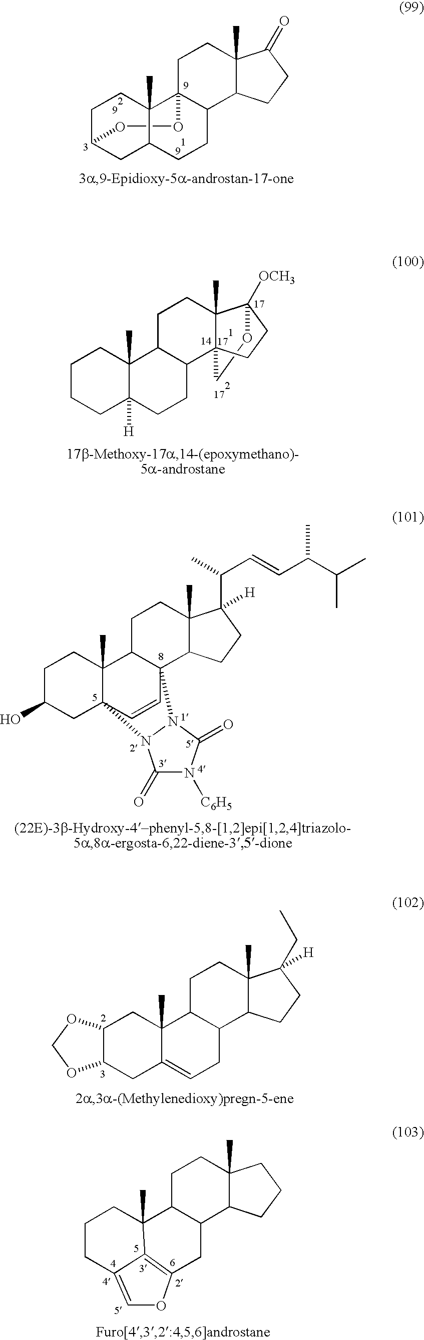

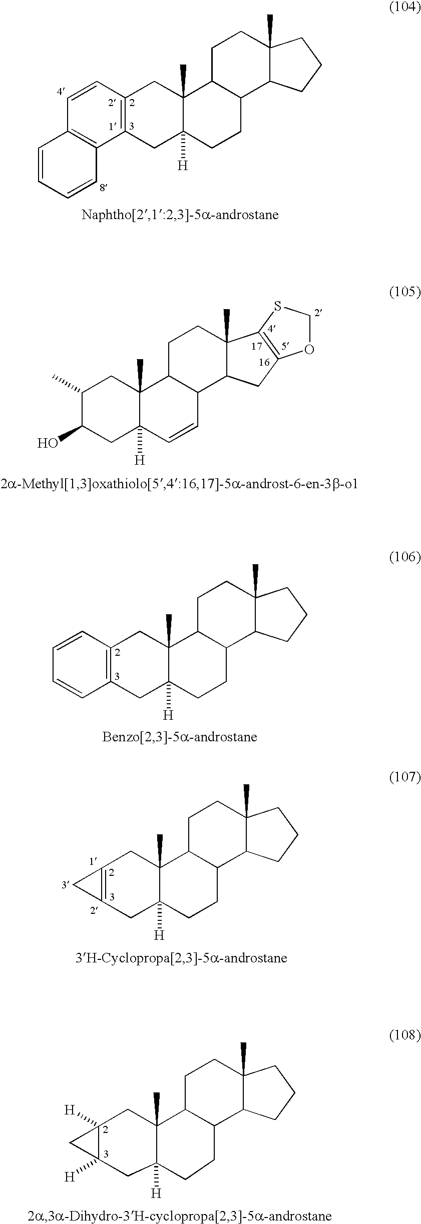

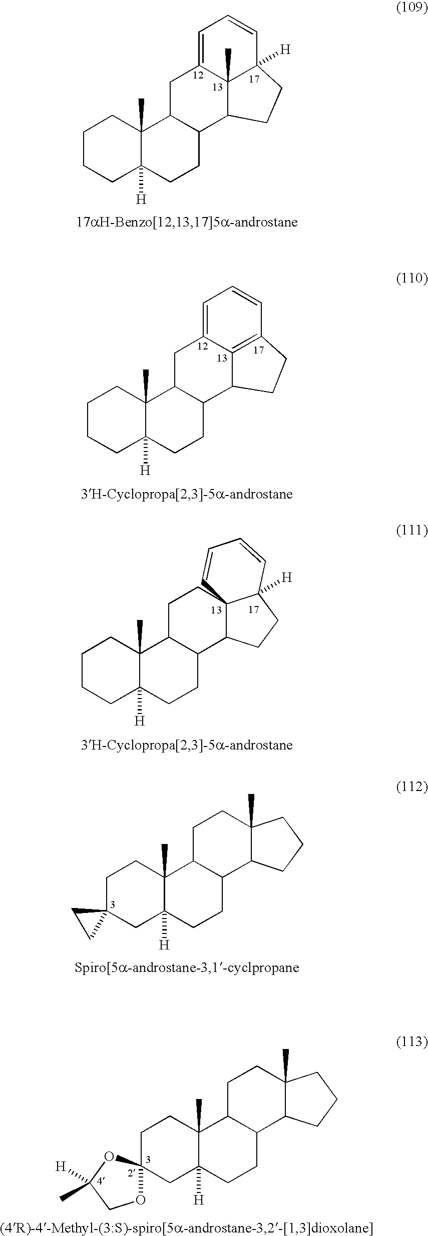

Yet, in certain embodiments, two or more steroid molecules are linked together covalently, as exemplified by formulae 3a and 3b.

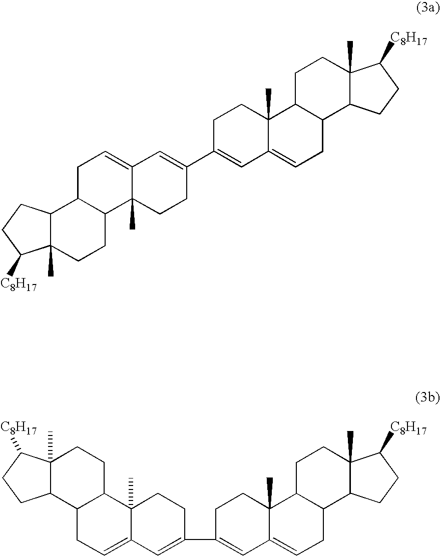

Table 3 provides examples of steroids that are useful according to the present invention.

TABLE 3
Exemplary steroids that are useful according to the present invention.

| | | Molecular |
|---|---|---|
| Trivial name | Chemical name | formula |
| Acrihellin | 5,14-dihydroxy-3^{β}-[(3-methylcrotonoyl)oxy]-19-oxo-5^{β}- | C₂₉H₃₈O₇ |
| | bufa-20,22-dienolide | |
| Actodigin | 3^{β}-(^{β}-D-glucopyranosyloxy)-14-hydroxy-24-nor-5^{β},14^{β}- | C₂₉H₄₄O₉ |
| | chol-20(2)-eno-21,23-lactone | |
| Alfacalcidol | (5Z,7E)-(1S,3R)-9,10-secocholesta-5,7,10(19)-triene-,3- | C₂₇H₄₄O₂ |
| | diol | |
| Betamethasone | 9-fluoro-11^{β},17,21-trihydroxy-16^{β}-methylpregna-1,4- | C₂₂H₂₉FO₅ |
| | diene-3,20-dione | |
| Canrenone | 3-oxo-17α-pregna-4,6-diene-21,17-carbolactone | C₂₂H₂₈O₃ |
| Clomegestone | 6-chloro-17-hydroxy-16α-methylpregna-4,6-diene-3,20- | C₂₂H₂₉ClO₃ |
| | dione | |
| Cyproterone | 6-chloro-1^{β},2^{β}-dihydro-17-hydroxy-3′H- | C₂₂H₂₇ClO₃ |
| | cyclopropa[1,2]pregna-4,6diene-3,20-dione | |
| Dexamethasone | 9-fluoro-11^{β},17,21-trihydroxy-16α-methylpregna-1,4- | C₂₂H₂₉FO₅ |
| | diene-3,20-dione | |
| Disogluside | (25R)-3^{β}-(^{β}-D-glucopyranosyloxy)spirost-5-ene | C₃₃H₅₂O₈ |
| Ethinylestradiol | 19-nor-17α-pregna-1,3,5(10)-trien-20-yne-3,17-diol | C₂₀H₂₄O₂ |
| Fluazacort | 21-acetoxy-9-fluoro-11^{β}-hydroxy-2′-methyl-16bH- | C₂₅H₃₀FNO₆ |
| | oxazolo[5′,4′:16,17]pegna--1,4-diene-3,20-dione | |
| Fluocortin | 6α-fluoro-11^{β}-hydroxy-16α-methyl-3,20-dioxopregna- | C₂₂H₂₇FO₅ |
| | 1,4-dien-21-oic acid | |
| Fusidic Acid | (17Z)-ent-16α-acetoxy-3^{β},11^{β}-dihydroxy-4^{β},8,14- | C₃₁H₄₈O₆ |
| | trimethyl-18-nor-5^{β},10α-cholesta--17(20),24-dien-21- | |
| | oic acid | |
| Gestrinone | 17-hydroxy-18α-homo-19-nor-17α-pregna-4,9,11-trien | C₂₁H₂₄O₂ |
| | 20-yn-3-one | |
| Halometasone | | |
| | 2-chloro-6α,9-difluoro-11^{β},17,21-trihydroxy-16α- | C₂₂H₂₇ClF₂O₅ |
| | methylpregna-1,4-diene-3,20-dione | |
| Hydrocortisone | 11^{β},17,21-trihydroxypregn-4-ene-3,20-dione | C₂₁H₃₀O₅ |
| Mebolazine | 17^{β}-hydroxy-2α,17-dimethyl-5α-androstan-3-one azine | C₄₂H₆₈N₂O₂ |
| Medroxyprogesterone | 17-hydroxy-6α-methylpregn-4-ene-3,20-dione | C₂₂H₃₂O₃ |
| Meproscillarin | 3^{β}-(6-deoxy-4-O-methyl-α-L-mannopyranosyloxy)-14- | C₃₁H₄₄O₈ |
| | hydroxybufa-4,20,22-rienolide | |
| Mespirenone | 7α-acetylthio-15α,16α-dihydro-3-oxo-3′H- | C₂₅H₃₀O₄S |
| | cyclopropa[15,1]-17α-pregna--1,4-diene-21,17- | |
| | carbolactone | |
| Mestranol | 3-methoxy-19-nor-17α-pregna-1,3,5(10)-trien-20-yn-17- | C₂₁H₂₆O₂ |
| | ol | |
| Naflocort | 9-fluoro-1′,4′-dihydro-11^{β},21-dihydroxy-16bH- | C₂₉H₃₃FO₄ |
| | naphtho[2′,3′:16,17]prena--1,4-diene-3,20-dione | |
| Norenthisterone | 17-hydroxy-19-nor-17α-pregn-4-en-20-yn-3-one | C₂₀H₂₆O₂ |
| Norgesterone | 17-hydroxy-19-nor-17α-pregna-5(10),20-dien-3-one | C₂₀H₂₈O₂ |
| Norgestrel | rac-17-hydroxy-18α-homo-19-nor-17α-pregn-4-en-20- | C₂₁H₂₈O₂ |
| | yn-3-one | |
| Oxandrolone | 17^{β}-hydroxy-17α-methyl-2-oxa-5α-androstan-3-one | C₁₉H₃₀O₃ |
| Oxymetholone | 17^{β}-hydroxy-2-(hydroxymethylene)-17α-methyl-5α- | C₁₉H₂₈O₃ |
| | androstan-3-one | |
| Pancuronium | 1,1′-(3α,17^{β}-diacetoxy-5α-androstane-2^{β},16^{β}-diyl)bis(- | C₃₅H₆₀Br₂N₂O₄ |
| bromide | methylpiperidinium) dibromide | |
| Prednisolone | 11^{β},17,21-trihydroxypregna-1,4-diene-3,20-dione | C₂₁H₂₈O₅ |
| Prednisone | 17,21-dihydroxypregna-1,4-diene-3,11,20-trione | C₂₁H₂₆O₅ |
| Proscillardin | 3^{β}-(6-deoxy-α-L-mannopyranosyloxy)-14-hydroxybufa- | C₃₀H₄₂O₈ |
| | 4,20,22-trienolide | |
| Roxibolone | 11^{β},17^{β}-dihydroxy-17α-methyl-3-oxoandrosta-1,4- | C₂₁H₂₈O₅ |
| | diene-2-carboxylic acid | |
| Spironolactone | 7α-acetylthio-3-oxo-17α-pregn-4-ene-21,17- | C₂₄H₃₂O₄S |
| | carbolactone | |
| Timobesone | S-methyl 9-fluoro-11^{β},17α-dihydroxy-16^{β}-methyl-3- | C₂₂H₂₉FO₄S |
| | oxoandrosta--1,4-diene-17^{β}-carbothioate | |
| Triamcinolone | 9-fluoro-11^{β},16α,17,21-tetrahydroxypregna-1,4-diene- | C₂₁H₂₇FO₆ |
| | 3,20-dione | |
| Ursodeoxycholic | 3α,7^{β}-dihydroxy-5^{β}-cholan-24-oic acid | C₂₄H₄₀O₄ |
| acid | | |

Mixtures of these steroids may also be employed according to the present invention.

The steroid is included in the composition of the present invention in a concentration that provides a desirable ratio between the efficacy and safety. Typically, steroids are included in the composition in a concentration between about 0.005% and about 12%. However, in some embodiments, the concentration is between about 0.005% and about 0.5%, in other embodiment between about 0.5% and about 2%, and in additional embodiments between about 2% and about 5% or between about 5% and about 12%.

In one or more embodiments, the steroid possesses immunomodulating and/or anti-inflammatory properties. Without being bound to a specific theory, immunomodulating and/or anti-inflammatory steroids act, among other mechanisms, through inhibition of the activity of phospholipase A₂. They also may have anti-proliferative effects on keratinocytes and other cell types. They can suppress collagen synthesis by fibroblasts, but this may lead to adverse effects. Anti-inflammatory steroids are roughly grouped according to relative anti-inflammatory activity, but activity may vary considerably depending upon the vehicle, the site of application, disease, the individual patient and whether or not an occlusive dressing is used, as exemplified in the Table 4.

TABLE 4
Exemplary anti-inflammatory steroids that are useful according to the
present invention.

| | | Typical concentration |
|---|---|---|
| Relative Potency | Generic Name | in topical products |
| Low Potency | Hydrocortisone | 0.5%-1% |
| | hydrocortisone acetate | 0.5-1.0% |
| | Desonide | 0.02-0.2% |
| Medium Potency | Betamethasone valerate | 0.05%-0.1% |
| | Prednicarbate | 0.02-0.2% |
| | Clobetasone-17-butyrate | 0.05% |
| | Flucinonide | 0.01%-0.05% |
| | Fluocinolone acetonide | 0.01-0.01% |
| | Alcometasone dipropionate | 0.01% |
| | Mometasone furoate | 0.1% |
| | Triamcinolone acetonide | 0.025%-0.1% |
| High Potency | Betamethasone-17-benzoate | 0.025% |
| | Methylprednisolone aceponate | 0.1% |
| | Betamethasone dipropionate | 0.025%, 0.05% |
| | Halcinonide | 0.1% |
| | Triamcinolone acetonide | 0.5% |
| Highest Potency | Halobetasol | 0.05% |
| | Clobetasol-17-propionate | 0.05% |

In one or more embodiments, the steroid is selected from the group of low-potency anti-inflammatory steroids, medium potency anti-inflammatory steroids and high potency anti-inflammatory steroids.

In one or more embodiments, the anti-inflammatory steroid is included in the composition at a concentration between about 0.005% and about 1%.

The McKenzie vasoconstrictor assay, as described, for example, in the British Journal of Dermatology 1975; 93:563-71 and versions thereof, has been the primary method used for classifying the potency of a product, containing an anti-inflammatory steroids. Thus, in one or more embodiments, the anti-inflammatory steroid is a steroid that positively affects the vasoconstrictor assay.

In one or more embodiments, the steroid is a hormone. Hormones are known to affect a variety of biological processes in any organism, and thus, their inclusion in the composition of the present invention, which is intended for local treatment of the skin, the vagina, the rectum as well as other body surfaces and cavities provided an advantageous treatment modality. Such compositions containing hormones can be further administered systemically, via the transdermal or transmucosal route, in order to alleviate a disorder that is affected by the specific hormone, or in order to tune the hormonal balance of the body in order to attain certain effects controlled by hormones, such as contraception and birth induction.

In one or more embodiments, the steroid hormone is a male hormone or an androgen. Non-limiting examples of male hormones/androgens include testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone.

In one or more embodiments, the steroid hormone is a female hormone or an estrogen. Non-limiting examples of female hormones/estrogens include estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin.

In one or more embodiments, the steroid hormone is a progestogen. Non-limiting examples of progestogens include progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate.

Yet, in additional embodiments, the steroid an inhibitor of a steroid hormone. Non-limiting examples of such inhibitors are finasteride, dutasteride and spironolactone.

In one or more embodiments, the steroid is a vitamin D. The term vitamin D is used to describe all steroids that exhibit qualitatively the biological activity of calciol (vitamin D₃). Non limiting examples of vitamin D compounds are provided in Table 5.

Yet, in additional embodiments, the steroid is a vitamin D₃ analogue. Exemplary vitamin D₃ analogs include calcipotriol, tacalcitol, maxacalcitol, and calcitriol, with calcipotriol being especially preferred. Vitamin D₃ analogues and derivatives are known to degrade at low pH levels. Therefore, in certain preferred embodiments, the steroid is a vitamin D₃ or an analogue or a derivative thereof, the pH is adjusted to the range between about 7 and about 10, or between about 7.5 and about 9. In one or more embodiments, the pH is adjusted using a buffering agent, suitable of maintaining a pH level between about 7 and about 10, or between about 7.5 and about 9.

TABLE 5
Examples of vitamin D compounds

| Vitamin D name | Systematic steroid name |
|---|---|
| Cholecalciferol (also termed calciol, | (5Z,7E)-(3S)-9,10-seco-5,7,10(19)- |
| cholecalciferol, vitamin D₃ and colecalciferol) | cholestatrien-3-ol |
| 25-Hydroxycholecalciferol (also termed | (5Z,7E)-(3S)-9,10-seco-5,7,10(19)- |
| calcidiol | cholestatriene-3,25-diol |
| 1α,25-Dihydroxycholecalciferol (also termed | (5Z,7E)-(1S,3R)-9,10-seco-5,7,10(19)- |
| calcitriol) | cholestatriene-1,3,25-triol |
| Ergocalciferol (also termed ercalciol and | (5Z,7E,22E)-(3S)-9,10-seco-5,7,10(19),22- |
| ergocalciferol) | ergostatetraen-3-ol |
| 1α,25-Dihydroxyergocalciferol (also termed | (5Z,7E,22E)-(1S,3R)-9,10-seco- |
| ercalcitriol) | 5,7,10(19),22-ergostatetraen-1,3,25-triol |
| 22,23-Dihydroergocalciferol (also termed | (5Z,7E)-(3S)-9,10-seco-5,7,10(19)- |
| (24S)-methylcalciol and 22,23- | ergostatrien-3-ol |
| dihydroercalciol) | |
| 1α,24R,25-Trihydroxycholecalciferol (also | (5Z,7E)-(1S,3R,24R)-9,10-seco-5,7,10(19)- |
| termed calcitetrol) | cholestatriene-1,3,24,25-tetrol |
| Previtamin D₃ (also termed precalciferol and | (6Z)-(3S)-9,10-seco-5(10),6,8-cholestatrien- |
| (6Z)-tacalciol) | 3-ol |
| Tachysterol₃ (also termed tacalciol) | (6E)-(3S)-9,10-seco-5(10),6,8-cholestatrien- |
| | 3-ol |
| Isovitamin D₃ (also termed (5E)-isocalciol) | (5E,7E)-(3S)-9,10-seco-1(10),5,7- |
| | cholestatrien-3-ol |
| Dihydrotachysterol₃ (also termed | (5E,7E)-(3S,10S)-9,10-seco-5,7- |
| dihydroercalciol) | cholestadien-3-ol |

Further examples of vitamin D compounds include, but are not limited to (1S)-Hydroxycalciol (also termed 1α-hydroxycholecalciferol and alfacaleidol), (24R)-Hydroxycalcidiol (also termed 24(R),25-dihydroxycholecalciferol), 25-Fluorocalciol (also termed 25-fluorocholecalciferol), Ercalcidiol (also termed 25-hydroxyergocalciferol), Ertacalciol (also termed tachysterol₂, (5E)-lsocalciol (also termed isovitamin D₃, 22,23-Dihydroercalciol), (24S)-methylcalciol (also termed vitamin D₄), (5E)-(10S)-10,19-Dihydroercalciol, (also termed dihydrotachysterol₂, hytakerol, and dihydrotachysterol), (24S)-Ethylcalciol (also termed vitamin D₅) and (22E)-(24R)-Ethyl-22,23-didehydrocalciol, (also termed vitamin D₆).

In one or more embodiments, the steroid is a phytosteroid or a phytosterol. As used herein, the term “phytosteroid” or “phytosterol” includes all steroids that are obtained, derived or extracted from plant sources. Non-limiting examples of families of phytosteroids and phytosterols include ecdysones, withanolids, sterines, steroid saponins and soflavonoids. Non-limiting examples of phytosteroid and phytosterol compounds include alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol, as well as all natural or synthesized forms and derivatives thereof, such as fatty acid esters, such as ferulic acid esters, oleoyl esters, and cinnamic acid esters, including isomers.

Plant oils and extracts which contain steroids are also useful. Non limiting examples of plants that contain steroids include nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis

In one or more embodiments, the steroid is a compound that is positively identified using a laboratory method, suitable of detecting a steroid. Anti-inflammatory or anti-allergic agents

Yet, according to another embodiment according to the present invention the active agent is an anti-inflammatory or anti-allergic agent. Anti-inflammatory agents can be selected from the group of corticosteroids, non-steroidal anti-inflammatory drugs (NSAIDs), anti-histamines, immunosuppressant agents, immunomodulators; and any combination thereof at a therapeutically effective concentration.

The following table provides a summary of currently available corticosteroid agent and their typical therapeutically effective concentration.

| Potency | Compound | Current products |
|---|---|---|
| Very high | Clobetasol proprionate | Cream or ointment 0.05% |
| | Halobetasol proprionate | Cream or ointment 0.05% |
| High | Betamethasone diproprionate | Cream or ointment 0.05% |
| | Betamethasone valerate | Ointment 0.1% |
| | Fluocinolone acetonide | Cream 0.02% |
| | Halcinonide | Cream or ointment 0.1% |
| Medium | Betamethasone valerate | Cream 0.1% |
| | Fluocinolone acetonide | Cream or ointment 0.020% |
| | Hydrocortisone valerate | Cream or ointment 0.2% |
| | Triamcinolone acetonide | Cream, ointment, or lotion |
| | | 0.1% or 0.020% |
| Low | Hydrocortisone | Cream, ointment, or lotion |
| | | 1.0% or 2.5% |

The concentrations of corticosteroid drugs, as presented in the above table are provided herein only as example, and any therapeutically effective concentration of such corticosteroids can be incorporated in the composition of the present invention.

Since corticosteroid drugs are typically hydrophobic, the composition of the present invention, comprising a hydrophobic solvent, is most suitable as a vehicle to facilitate better topical distribution, improved occlusion and an enhanced rate of penetration of any of the above listed drugs.

Corticosteroids are used for treating psoriasis. Psoriasis is a very common chronic skin disease, which may be the target of treatment using the composition of the present invention. It is marked by periodic flare-ups of sharply defined red patches covered by a silvery, flaky surface.

Corticosteroid ointments, greasy preparations containing little or no water, are typically used for treating psoriasis. Their main disadvantage is in their sticky feeling, which remains so long after treatment is over. By contrast, the foam of the present invention, while comprising considerable concentration of an oil (hydrophobic solvent), spreads very easily throughout the afflicted area and absorbs into the skin without leaving any untoward sensation or look.

Other non-limiting examples of inflammatory disorders, which can be prevented or treated by the oleaginous compositions of the present invention, wherein the drug is a steroid are atopic dermatitis, seborrhea, seborrheic dermatitis of the face and trunk, seborrheic blepharitis, contact dermatitis, stasis dermatitis (gravitational eczema; varicose eczema), exfoliative dermatitis (erythroderma), lichen simplex chronicus, pityriasis rosea and pemphigus.

It is pointed out that certain of the solvents that may be used in the preparation of the composition of the present invention including polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA) are themselves beneficial in the treatment of psoriasis and other skin inflammation conditions.

A second class of anti-inflammatory agents, which is useful in the foam of the present invention, includes the nonsteroidal anti-inflammatory agents (NSAIDs). The variety of compounds encompassed by this group is well-known to those skilled in the art. Specific non-steroidal anti-inflammatory agents useful in the composition invention include, but are not limited to:

Oxicams, such as piroxicam, isoxicam, tenoxicam, sudoxicam;

Salicylates, such as salicylic acid, ethyl salicylate, methyl salycilate, aspirin, disalcid, benorylate, trilisate, safapryn, solprin, diflunisal, and fendosal;

Acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;

Fenamates, such as mefenamic, meclofenamic, flufenamic, niflumic, and tolfenamic acids;

Propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and

Pyrazoles, such as phenylbutazone, oxyphenbutazone, feprazone, azapropazone, and trimethazone.

Any further steroidal and nonsteroidal compounds, having the capacity to prevent, alleviate the symptoms of, treat or cure inflammation processes, are generally included, as possible anti-inflammatory agents, according to the present invention.

Topical antihistaminic preparations currently available include 1% and 2% diphenhydramine, 5% doxepin, phrilamine maleate, chlorpheniramine and tripelennamine, phenothiazines, promethazine hydrochloride and dimethindene maleate. These active agents, as well as additional antihistamines can also be incorporated in the composition of the present invention.

The therapeutic composition of the present invention may also comprise an anti-inflammatory or antiallergic agent, wherein said agent reduces the occurrence of pro-inflammatory cytokines or inhibits the effect of pro-inflammatory cytokines. Mixtures of such anti-inflammatory agents may also be employed, as well as the dermatologically acceptable salts, esters, amides, prodrugs and derivatives of these agents.

Non-steroidal Antiinflammatory Agents (NSAID)

In the context of the present invention, a nonsteroidal immunomodulating agent (also termed herein “nonsteroidal anti-inflammatory agent” and “NSAID”) is a pharmaceutically active compound, other than a corticosteroid, which affects the immune system in a fashion that results in a reduction, inhibition, prevention, amelioration or prevention of an inflammatory process and/or the symptoms of inflammation and or the production pro-inflammatory cytokines and other pro-inflammatory mediators, thereby treating or preventing a disease that involves inflammation.

In one or more embodiments, the NSAID is an inhibitor of the cyclooxygenase (COX) enzyme. Two forms of cyclooxygenase are known today: the constitutive cyclooxygenase (COX-1); and the inducible cyclooxygenase (COX-2), which is proinflammatory. Thus, in one or more embodiments of the present invention, the NSAID is selected from the group consisting of a COX-1 inhibitor, a COX-2 inhibitor or a non-selective NSAID, which simultaneously inhibits both COX-1 and COX-2.

The term “selective COX-2 inhibitor” relates o a compound able to inhibit cyclooxygenase-2 without significant inhibition of COXe-1. Typically, it includes compounds that have a COX-2 IC₅₀ of less than about 0.2 micro molar, and also have a selectivity ratio of COX-2 inhibition over COX-1 inhibition of at least 50, and more typically, of at least 100. Inhibitors of the cyclooxygenase pathway in the metabolism of arachidonic acid used in the present invention may inhibit enzyme activity through a variety of mechanisms. By the way of example, and without limitation, the inhibitors used in the methods described herein may block the enzyme activity directly by acting as a substrate for the enzyme.

Selective COX-2 Inhibitors include, in an exemplary manner diaryl-substituted furanones (e.g., Rofecoxib); diaryl-substituted pyrazoles (e.g., Celecoxib); indole acetic acids (e.g., Etodolac); and sulfonanilides (e.g., Nimesulide) and salts and derivatives thereof.

In one or more embodiments, the selective COX-2 inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)-phenyl]-3(2H)-pyridazinone, 2-[(2,4-dichloro-6-methylphenyl)amino]-5-ethyl-1-benzeneacetic acid, (3Z)-3-[(4-chlorophenyl)[4-(methylsulfonyl)phenyl]met-hylene]dihydro-2(3H)-furanone, and (S)-6,8-dichloro-2-(trifluoromethyl)-2H—-1-benzopyran-3-carboxylic acid.

In additional embodiments, the selective COX-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, and 2-fluoro-a-methyl[1,1′-biphenyl]-4-ace-tic acid, 4-(nitrooxy)butyl ester.

In one or more embodiments, the NSAID is salicylic acid a salicylic acid derivatives. Exemplary salicylic acid derivative include, in a non limiting fashion, aspirin, sodium salicylate, choline magnesium trislicylate, salsalate, diflunisal, salicylsalicylic acid, sulfasalazine, olsalazine, esters of salicylic acid with a carboxylic acid, esters of salicylic acid with a dicarboxylic acid, esters of salicylic acid with a fatty acid, esters of salicylic acid with a hydroxyl fatty acid, esters of salicylic acid with an essential fatty acid, esters of salicylic acid with a polycarboxylic acid, and any compound wherein salicylic acid is linked to an organic moiety through a covalent bond.

In one or more embodiments, the NSAID is para-aminophenol (e.g., acetaminophen) and salts and derivatives thereof.

In one or more embodiments, the NSAID is an indole or an indole—acetic acid derivative (e.g., indomethacin, sulindac, etodolac) and salts and derivatives thereof.

In one or more embodiments, the NSAID is an aryl acetic acids (e.g., tolmetin, diclofenac, ketorolac) and salts and derivatives thereof.

In one or more embodiments, the NSAID is an arylpropionic acid and salts and derivatives thereof. Exemplary arylpropionic acid derivative include, in a non limiting fashion, are ibuprofen, naproxen, flubiprofen, ketoprofen, fenoprofen, oxaprozin.

In one or more embodiments, the NSAID is anthranilic acids or an anthranilic acid derivative, also termed “fenamates” (e.g., mefenamic acid, meclofenamic acid) and salts and derivatives thereof.

In one or more embodiments, the NSAID is selected from the group of enolic acids, enolic acid salts, enolic acid esters, amides, anhydrides and salts and derivatives thereof. Non-limiting examples of enolic acid derivatives include oxicams (piroxicam, tenoxicam) and pyrazolidinediones (phenylbutazone, oxyphenthratrazone).

Yet, in additional embodiments, the NSAID is an alkanone (e.g., nabumetone).

Certain imidazole drugs (e.g., ketoconazole) also possess anti-inflammatory properties. (See: J. Am. Acad. Dermatol. 1991 August; 25(2 Pt 1):257-61).

Another group of nonsteroidal immunomodulating agents includes agents, which inhibit pro-inflammatory cytokines, such as TNF-alpha, TNF-beta, interleukin-1, interleukin-4, interleukin-6, interleukin-10, interleukin-12 and IFN-gamma from T cells, which are especially important in the induction of inflammation or inhibit the release of cytokines and pro-inflammatory mediators from mast cells.

Agents that are used to affect the untoward influence of pro-inflammatory cytokines are chemically or biologically-originated materials that suppress the pro-inflammatory effect of a pro-inflammatory cytokine via various mechanisms, including, but not limited to (a) inhibiting the formation of a pro-inflammatory cytokine; (b) suppressing the interaction of a pro-inflammatory cytokine with its receptors; or (c) neutralization the proinflammatory cytokine by direct or indirect interaction.

Examples of chemical anti TNF-α agents are known pharmaceutical materials, such as pentoxifylline, propentofylline, torbafylline (and other related xanthines), amiloride, chloroquine, thalidomide and structural analogs thereof. Examples for biological anti-TNF-α agents are anti-TNF-α antibodies and soluble TNF-α receptors. Additional compounds are those that impair the signal transduction cascade from the receptor to other functional organs of the living cell. Such active agents, as well additional compounds, which are capable of inhibiting the production or otherwise suppressing the pro-inflammatory effects of TNF-α can be used in the composition of the present invention.

Immunosuppressant agents, immunoregulating agents and immunomodulators constitute an additional class of nonsteroidal anti-inflammatory agents, which are used according to the present invention. Such agents are chemically or biologically-derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod. In one or more embodiments, the non steroidal immunomodulating agent is a calcineurin Inhibitor.

In one or more embodiments, the NSAID is a nitric oxide inhibitor. Nitric oxide (NO) is a potent secondary messenger that is both highly reactive and highly diffusible. It is generated physiologically by a family of enzymes, referred to as NO synthases (NOS). Overproduction of NO plays a key role in the pathology of a wide range of disorders including disorders that involve inflammation, and NOS inhibitors have been suggested as anti-inflammatory agents. Agents that neutralize NO (also called “NO scavengers”) are considered as potential anti-inflammatory agents as well.

Also useful are compounds that inhibit or slow down the migration of leucocytes (white blood cell), e.g., macrophages, neutrophils, and monocytes towards an afflicted skin surface or mucosal membrane, which is known to accelerate the inflammatory process.

Among other inhibitors of leucocyte chemoaxis, dicarboxylic acids, having between about 6 and about 14 carbon atoms in their carbon atom skeleton are particularly useful in the treatment of disorders of the skin and mucosal membranes that involve inflammation. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments of the present invention, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred.

Certain preferred dicarboxylic acid derivatives include a dicarboxylic acid wherein at least one ester moiety of the compound comprises a keratolytic agent, selected from the group consisting of alpha-hydroxy acids and derivatives thereof, beta-hydroxy acids and derivatives thereof, hydroxybenzoic acid and their ester, anhydride and amine derivatives, alkylhydroxybenzoate, dihydroxy benzene and their ester, anhydride and amide derivatives, cresols and their ester, anhydride and amide derivatives. Keratolytic agents also include alcohol derivatives of Vitamin A (retinoic acid), e.g., retinol and derivatives thereof, as provided in U.S. Pat. No. 6,180,669. Additional preferred dicarboxylic acid derivatives comprise at least one ester of a active alcohol moiety, selected from the groups of steroid hormones, corticosteroids, vitamin E and vitamin D, as provided in US Patent Application 20040191196.

In one or more embodiments, the NSAID is an ion channel modulator. Ion channels are protein macromolecules located in the cell membranes that enable the selective movement of sodium, potassium, and calcium from outside the cell to inside the cell and vice-versa.

In one or more embodiments, the NSAID is a potassium ion channel modulator. It has been shown that the potassium ion channel modulator play important roles in controlling T-cell activation and thus, they can be used to control inflammation.

In one or more embodiments, the potassium ion channel modulator is selected from the group consisting of dendrotoxin, dendrotoxin 1, dendrotoxin K, alpha-dendrotoxin, beta-dendrotoxin, gamma-dendrotoxin, margatoxin, stichodactyla toxin, tityustoxin K, apamin, charylotoxin, clotrimazole, dequalinium chloride, iberiotoxin, kaliotoxin, neuropeptide Y, noxiustoxin, tolbutamide, chlorpropamide, glibenclamide, glipizide, nategliniide, repagliniide, glyburide, tolazamide, nicorandil, fampridine and penitrem A, or is a pharmaceutically acceptable salt or prodrug thereof.

In an embodiment of the present invention, the potassium ion channel modulator is selected from the list of potassium ion channel modulators, provided in WO 2004/093895.

In one or more embodiments, the NSAID is a sodium ion channel modulator. In one or more embodiments, the sodium ion channel blocker is selected from the group consisting of disopyramide, procainimide, quinidine, tocamide, mexiletene, lidocane, phenyloin, fosphenyloin, flecamide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encamide, and flunarizine or is an isomer, a pharmaceutically acceptable salt, ester, or prodrug thereof.

In an embodiment of the present invention, the potassium ion channel modulator is selected from the list of potassium ion channel modulators, provided in US Pat. Pub. Nos. 20040224940 and 20040220187.

In one or more embodiments, the NSAID is a modulator of serotonin (5-hydroxytryptamine, 5-HT) activity. 5-HT is known to affect inflammation through its modulation effect on cytokine production (Cloëz-Tayarani et al. Int. Immunol. 2003, 15 233). In certain embodiments, the serotonin activity modulator is a serotonin reuptake inhibitor. It has been shown that serotonin reduces inflammation and assists healing of experimental skin wounds, and thus, serotonin reuptake inhibitor can be used to control inflammation and associated disorders.

In one or more embodiments, the serotonin reuptake inhibitor is selected from the group consisting of citalopram, fluoxetine, fluvoxamine, paroxetine, escitalopram oxalate, sertraline, norfluoxetine and N-demethylsertraline.

In an embodiment of the present invention, the serotonin reuptake inhibitor is selected from the list of potassium ion channel modulators, provided in US Pat Pub. No.

20040171664.

In one or more embodiments, the NSAID is an antioxidant. Reactive oxygen species play an important role in mediating skin inflammation, and antioxidants may provide protection.

Non-limiting examples of antioxidant agents include 21-[4-[2-amino-6-(diethylamino)-4-pyrimidinyl]-1-piperazinyl]-17α-hydroxypregna-4,9(11)-diene-3,20-dione, 17α-hydroxy-21-[4-[2,6-bis(dimethylamino)-4-pyrimidinyl]-1-piperazinyl]pregna-4,9(11)-diene-3,20-dione, 21-[4-[2-(diethylamino)-6-(1-pyrrolidinyl)-4-pyrimidinyl]-1-piperazinyl]-17α-hydroxypregna-4,9(11)-diene-3,20-dione, 17α-hydroxy-21-[4-[2-(diethylamino)-6-(4-methyl-1-piperazinyl(4-pyrimidinyl)]-1-piperazinyl]pregna-4,9(11)-diene-3,20-dione, 17α-hydroxy-21-[4-[2,6-bis(diethylamino)-4-pyrimidinyl]1-piperazinyl]pregna-4,9(11)-diene-3,20-dione, 1α-hydroxy-21-[4-[2-(diethylamino-)-6-(1-piperidinyl)-4-pyrimidinyl]-1-piperazinyl]pregna-4,9(11)-diene-3,20-dione, 21-[4-[2,6-bis(diethylamino)-b4-pyrimidinyl)-4-pyrimidinyl]-1-piperazinyl]-1-piperazinyl]-17α-hydroxy-16α-methylpregna-1,4,9(11)-triene-3,20-dione, 17α-hydroxy-21-[4-[2,6-bis(4-methyl-1-piperazinyl]pregna-4,9(11)-diene-3,20-dione, 17α-hydroxy-6α-methyl-21 [4-2,6-bis-(1-pyrrolidinyl-4-pyrimidinyl]-1-piperazinyl]pregna-1,4,9(11)-triene-3,20-dione, 21-[4-2,6-bis(diethylamino)-4-pyrimidinyl]-1-piperazinyl]-1-1α,17α-dihydroxypregn-4-ene-3,20-dione, 21-[4-[2,6-bis(diethylamino)-4-pyrimidinyl[-1 piperazinyl]-17α-hydroxypregn-4-ene-3,20-dione, 21-[4-[2,6-bis(diethylamino)-4-pyrimidinyl]-1-piperazinyl]-17α-hydroxy-6α-methylpregna-1,4,9(11)-triene-3,20-dione, 17α-hydroxy-21-[4-[2,6-bis(1-pyrrolidinyl)-4-pyrimidinyl]-1-piperazinyl]pregna-4,9(11)-diene-3,20-dione, 21-[4-[2,6-bis(diethylamino)-4-pyrimidinyl]-1-piperazinyl]-11α-hydroxypregn-4-ene-3,20-dione, 21-[4-[2,6-bis(diethylamino)-4-pyrimidinyl]-1-piperazinyl]-11α,17α-dihydroxypregn-4-ene-3,20-dione, 17α-hydroxy-16α-methyl-21-[4-[2,6-bis-(1-pyrrolidinyl)-4-pyrimidinyl]-1-piperazinyl]pregna-1,4,9(-11)-triene-3,20-dione17α-hydroxy-21-[4-[2,6-bis(1-pyrrolidinyl)-4-pyrimidinyl]-1-piperazinyl]pregna-1,4,9(11)-triene-3,20-dione, 21-[4-[2,6-bis(diethylamino)-4-pyrimidinyl]-1-piperazinyl]-17α-hydroxypregna-1,4,9(11)-triene-3,20-dione, 21-[4-[4,6-bis(diethylamino)-2-pyrimidinyl]-1-piperazinyl]-17α-hydroxypregna-1,4,9(11)-triene-3,20-dione, 21-[4-[2,6-bis(diethylamino)-4-pyrimidinyl]-1-piperazinyl]-16α-methylpregna-1,4,9(11)-triene-3,20-dione, 21-[4-[2,6-bis(diethylamino)-4-pyrimidinyl]-1-piperazinyl]-11.alpha.-hydroxy-16α-methylpregna-1,4-dien-e-3,20-dione, 21-[4-[2,6-bis(diethylamino)-4-pyrimidinyl]-1-piperazinyl]-1-6α-methylpregna-1,4-diene,3,20-dione, 16α-methyl-21-[4-[2,6-bi-s(1-pyrrolidinyl)-4-pyrimidinyl]-1-piperazinyl]pregna-1,4,9(11)-triene-3,2-O-dione, 11α-hydroxy-16α21-[4-[2,6-bis(1-pyrrolidinyl)-4-pyrimidinyl]piperazinyl]pregna-1,4-diene-3,20-dione, 16α-methyl-21-[4-[2,6-bis(1-pyrrolidinyl)-4-pyrimidinyl]-1-piperazinyl]pregna-1,4-diene-3,20-dione, 16.alpha.-methyl-21-[4-[2,6-bis(4-morpholino)-4-pyrimidinyl]-1-piperazinyl]pregna-1,4,9(11)-triene-3,20-dione, 11α-hydroxy-16α-methyl-21-[4-[2,6-bis(4-morpholino)-4-pyrimidinyl]-1-piperazinyl]pregna-1,4-diene-3,20-dione, 16.alpha.-methyl-21-[4-[2-,6-bis(4-morpholino(4-pyrimidinyl]-1-piperazinyl]pregna-1,4-diene-3,20-dione, 21-[4-[2,6-bis(allylamino)-4-pyrimidinyl]-1-piperazinyl[-16α-methylpregna-1,4,9(11)-triene-3,20-dione, 21-[4-[2,6-bis(allylamino)-4-pyrimidinyl]-1-piperazinyl]-11α-hydroxy-16α-methylpregna-1,4-ene-3,2-O-dione, 21-[4-[2,6-bis(allylamino)-4-pyrimidinyl]-1-piperazinyl]-16α-methylpregna-1,4-ene-3,20-dione, 21-[4-[2,6-bis(1-pyrrolidinyl)-4-pyrimidinyl]-1-piperazinyl]pregn-4-ene-3,11,20-trione, 21-[4-[2,6-bis(1-pyrrolidinyl)-4-pyrimidinyl]-1-piperazinyl]pregna-4,9(11)-diene-3,20-dione, 21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]pregna-4,9(11)-diene-3,20-dione, 21-[4-(2,6-bis(4-morpholino)-4-pyrimidinyl)-1-piperazinyl]-17α-hydroxypregna-4,9(11)-diene-3,20-dione, 21-[4-(2,6-bis(1-pyrrolidinyl)-4-pyrimidinyl)-1-piperazinyl]pregna-4-en-3-one, 21-[4-(2,6-bis(1-pyrrolidinyl)-4-pyrimidinyl)-1-piperazinyl]pregn-4-en-3-one, 16α-methyl-21-[4-[2,6-bis(1-pyrrolidinyl)-4-pyrimidinyl]-1-piperazinyl]pregna-1,4,9(11)-triene-3,20-dione, 21-[4-(2,6-bis(1-pyrrolidinyl)-4-pyrimidinyl)-1-piperazinyl]pregna-1,4,9(11)-triene-3,20-dione, 21-[4-(2,6-bis(1-pyrrolidinyl)-4-pyrimidinyl)-1-piperazinyl]-20-methylpregna-1,4-dien-3-one, 21-[4-(2,6-bis(1-pyrrolidinyl)-4-pyrimidinyl)-1-piperazinyl]pregna-1,4,9(11),16-tetraene-3,20-dione, 21-[4-[2,6-bis(4-morpholino)-4-pyrimidinyl]-1-piperazinyl]pregna-1,4-diene-3,20-dione, 21-[4-[2,6-bis(diethylamino)-4-pyrimidinyl]-1-piperazinyl]-6α-fluoro-17α-hydroxy-16β-methylpregna-4,9(11)-diene-3,20-dione, 6α-fluoro-17α-hydroxy-16β-methyl-21-[4-[2,6-bis(1-pyrrolidinyl)-4-pyrimidinyl]-1-piperazinyl]pregna-4,9(11)-diene-3,20-dione, 16α-methyl-21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]-16α, 17α-dimethylpregna-1,4,9(11)-riene-3,2-O-dione, 3β-hydroxy-16α-methyl-21-[4-[2,6-bis(1-pyrrolidinyl)-4-1-pyrimidinyl]-1-piperazinyl]-pregn-5-en-20-one, 16α-methyl-21-[4-[2,-6-bis-(1-pyrrolidinyl)-4-pyrimidinyl]-1-piperazinyl]pregna-1,4,6,9(11)-tetraene-3,20-dione, 3β-hydroxy-16α-methyl-21-[4-[2,6-bis(1-pyrrolidinyl)-4-pyrimidinyl]-1-piperazinyl]pregn-5-en-20-one, 16α-methyl-17β-(1-oxo-4-[4-[2,6-bis(1-pyrrolidinyl)-4-pyrimidinyl]-1-piperazinyl]butyl)-androsta-4,9(11)-dien-3-one, tocopherol, vitamin C, beta-carotene, lycopene, coenzyme Q, idebenone, lipoic acid, and ginkgo biloba; or is an isomer, a pharmaceutically acceptable salt, ester, or prodrug thereof.

In one or more embodiments, the NSAID is a cannabinoid. Cannabnoids are known to affect inflammation through suppression of runaway inflammation and other untoward effects of immune system activation, as well as pain.

In certain embodiments, the cannabinoid agent is selected from the group consisting of: 2-arachidonylglycerol; N-arachidonyl-1-(2,3-dichlorobenzoyl)-2-methyl-3-(2-[1-morpholino]ethyl)-5-methoxyindole; 2-methyl-1-propyl-3-(1-naphthoyl)indole; 1-methoxy-N,N-dimethylmethanamide; 1-methoxy-endo-4-hydroxy-9-oxabicyclo(3.3.1)nonane; dronabinol; (2-methyl-1-propyl-1H-indol-3-yl)-1-naphthalenylmethanone; 3-(1,1-dimethylbutyl)-6a,7,10,10a-tetrahydro-6,6,9-6h-dibenzo[b,d]pyran; [2,3-dihydro-5-methyl-3(4-morpholinylmethyl)pyrrolo[1,2,3-de]methane; 5-(1,1-dimethylheptyl)-2-[(1R,2R,5R)-5-hydroxy-2-(3-hydroxypropyl)cyclohexyl]phenol; 5-(4-chlorophenyl)-1-(2,4-dichlorophenyl)-4-methyl-N-1-piperidinyl-1H-pyr-azole-3-caroxamide; [6-methoxy-2-(4-methoxyphenyl)benzo[b]furan-3-yl](4-cyanophenyl)methanone; [6-iodo-2-methyl-1-[2-(4-morpholinyl)ethyl]-1H-indol-3-yl](4-methoxy phenyl)methanone; 5-(4-chloro-3-methylphenyl)-1-[(4-methylphenyl)methyl]-N-(1,3,3-trimethyl-bicyclo[2.2.1]hept-2-yl)-(1S-endo)-1H-pyrazole-3-carboxamide; 1-(2,4-dichlorophenyl)-5-(4-iodophenyl)-4-methyl-n-1-piperidinyl-1H-pyraz-ole-3-carboxamide; 1-(2,4-dichlorophenyl)-5-(4-iodophenyl)-4-methyl-N-4-morpholinyl-1H-pyraz-ole-3-carboxamide; 3-(6-azido-2-hexynyl)-6a,7,10,10a-tetrahydro-6,6,9-trimethyl-(6aR,10aR)-6-H-dibenzo[b,d]pyran-1-ol; 3-[(2Z)-6-azido-2-hexynyl]-6a,7,10,10a-tetrahydro-6,6,9-trimethyl-(6aR,10-aR)-6H-dibenzo[b,d]pyran-1-ol; (−)-6,7-dichloro-1,4-dihydro-5-[3-(methoxymethyl)-5-(3-pyridinyl)-4H-1,2,-4-triazol-4-yl]-2,3-quinoxalinedione; (2R,4S)-rel-5,7-dichloro-1,2,3,4-tetrahydro-4-[[(phenylamino)carbonyl]ami-no]-2-quinolinecarboxylic acid; (2R,6S)-1,2,3,4,5,6-hexahydro-3-[(2S)-2-methoxypropyl]-6,11,11-trimethyl-2,6-methano-3-benzazocin-9-ol; (3E)-2-amino-4-(phosphonomethyl)-3-heptenoic acid; (3R,4S)-rel-3,4-dihydro-3-[4-hydroxy-4-(phenylmethyl)-1-piperidinyl]-2H-1-1-benzopyran-4,7-diol; (3S,4aR,6S,8aR)-decahydro-6-(phosphonomethyl)-3-isoquinoline carboxylic acid; (R)-9-bromo-2,3,6,7-tetrahydro-2,3-dioxo-N-phenyl-1H,5H-pyrido[1,2,-3-de]quinoxaline-5-acetamide; (.alpha.R)-.alpha.-amino-5-chloro-1-(phosphonomethyl)-1H-benzimidazole-2-propanoic acid; [2-(8,9-dioxo-2,6-diazabicyclo[5.2.0]non-1(7)-en-2-yl)ethyl]-phosphonic acid; [5-(aminomethyl)-2-[[[(5S)-9-chloro-2,3,6,7-tetrahydro-2,3-dioxo-1H,5H-py-rido[1,2,3-de]quinoxalin-5-yl]acetyl]amino]phenoxy]-acetic acid; 1,4-dihydro-6-methyl-5-[(methylamino)methyl]-7-nitro-2,3-quinoxaline-dion-e monohydrochloride; 1-[2-(4-hydroxyphenoxy)ethyl]-4-[(4-methylphenyl)methyl]-4-piperidinol hydrochloride; 1-[4-(1H-imidazol-4-yl)-3-butynyl]-4-(phenylmethyl)-piperidine; 1-aminocyclopentane-carboxylic acid (ACPC); 2-[(2,3-dihydro-1H-inden-2-yl)amino]-acetamide monohydrochloride; 2-hydroxy-5-[[(pentafluorophenyl)methyl]amino]-benzoic acid (PBAS); 2-methyl-6-(phenylethynyl)-pyridine (MPEP); 3-(phosphonomethyl)-L-phenylalanine; 3-[(1E)-2-carboxy-2-phenylethenyl]-4,6-dichloro-1H-indole-2-carboxylic acid; 4,6-dichloro-3-[(E)-(2-oxo-1-phenyl-3-pyrrolidinylidene)methyl]-1H-indole-2-carboxylic acid; 6-chloro-2,3,4,9-tetrahydro-9-methyl-2,3-dioxo-1H-indeno[1,2-b]pyrazine-9-1-acetic acid; 7-chlorothiokynurenic acid; 8-chloro-2,3-dihydropyridazino[4,5-b]quinoline-1,4-dione 5-oxide salt with 2-hydroxy-N,N,N-trimethyl-ethanaminium; aptiganel; besonprodil; budipine; conantokin G; delucemine; dexanabinol; felbamate; fluorofelbamate; gacyclidine; glycine; ipenoxazone; kaitocephalin; lanicemine; licostinel; midafotel; milnacipran; N′-[2-chloro-5-(methylthio)phenyl]-N-methyl-N-[3-(methylthio)phenyl]-guan-idine; N′-[2-chloro-5-(methylthio)phenyl]-N-methyl-N-[3-[(R)-methylsulfiny-l]phenyl]-guanidine; neramexane; orphenadrine; remacemide; topiramate; .alpha.-amino-2-(2-phosphonoethyl)-cyclohexanepropanoic acid; .alpha.-amino-4-(phosphonomethyl)-benzeneacetic acid; 8-[4-(1,1-dimethylheptyl)-2-hydroxyphenyl]decahydro-2-naphthalene methanol; 5,6,6a,7,8,9,10,10a-octahydro-6-methyl-3-[(1R)-1-methyl-4-pheny-l butoxy]-1,9-phenanthridinediol; Desacetyl-L-nantradol; R-(+)-methanandamide; 11-hydroxy-9,15-dioxoprosta-8,12,13-dienoic acid; 2-[3-methyl-6-(1-methylethenyl)-2-cyclohexen-1-yl]-5-pentyl-(1R-trans)-1,-3-benzenediol (cannabidiol); 3-amyl-1-hydroxy-6,6,9-trimethyl-6H-dibenzo[b,d]pyran (cannabinol); 3-(1,1-dimethylheptyl)-6a,7,8,9,10,10a-hexahydro-1-hydroxy-6,6-dimethyl-(-6aR,9R,10aR)-6H-dibenzo[b,d]pyran-9-methanol; 7-(1,1-dimethylheptyl)-1,2,3,4,4a,9b-hexahydro-2,2-dimethyl-4-methylene-1-,3-methanodibenzofuran-9-ol; 7-(1,1-dimethylheptyl)-1,2,3,4,4a,9b-hexahydro-2,2-dimethyl-4-methylene-1-(s),3-methanodibenzofuran-9-ol; 2-[4-[(acetyloxy)methyl]-6,6-dimethylbicyclo[3.1.1]hept-3-en-2-yl]-5-(1,1-dimethylheptyl)-diacetate[1R-(1a,2a,5a)]-1,3-benzenediol; 2-[4-[(acetyloxy)methyl]-6,6-dimethylbicyclo[3.1.1]hept-3-en-2-yl]-5-(1,1-dimethylheptyl)-diacetate[1S-(1a,2a,5a)]-1,3-benzenediol; 5-(1,1-dimethylheptyl)-2-[4-(hydroxymethyl)-6,6-dimethylbicyclo[3.1.1]hep-t-3-en-2-yl]-[1S-(1a,2a,5a)]-1,3-benzenediol; and 5-(1,1-dimethylheptyl)-2-[4-(hydroxymethyl)-6,6-dimethylbicyclo[3.1.1]hep-t-3-en-2-yl]-[1R-(1a,2a,5a)]-1,3-benzenediol; or is an isomer, a pharmaceutically acceptable salt, ester, or prodrug thereof.

In one or more embodiments, the NSAID is an angiotensin II receptor antagonist. angiotensin II receptor antagonists are known to affect inflammation and pain, as shown, for example in J Pharmacol Exp Ther. 2003 October; 307(1):17-23. Epub 2003 Aug. 27.

In certain embodiments, the angiotensin II receptor antagonist is selected from the group consisting of candesartan, eprosartan, irbesartan, losartan, olmesartan, tasosartan, telmisartan, valsartan, saralasin, and 1-[[4-(dimethylamino)-3-methylphenyl]methyl]-5-(diphenylac-etyl)-4,5,6,7-tetrahydro-1H-imidazo[4,5-c]pyridine-6-carboxylic acid ditrifluoroacetate, or an isomer, a pharmaceutically acceptable salt, ester, or prodrug thereof.

In one or more embodiments, the NSAID is an UDP-glucuronosyltransferase inhibitor (UGT inhibitor).

In certain embodiments, the UGT inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin and silymarin.

Mixtures of these non-steroidal immunomodulators may also be employed according to the present invention.

Topical application of a foam, comprising a safe and effective dose of an NSAID can be useful in the prevention and/or alleviation of the symptoms of rheumatoid arthritis, osteoarthritis and pain. Topical NSAIDs, incorporated in the foam of the present invention can be also used in the treatment of dermatological disorders, such as acne, rosacea, hair growth disorders, actinic keratosis and certain skin cancer conditions.

Immunosuppressant agents, immunoregulating agents and immunomodulators are chemically or biologically-derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod. Such compounds, delivered in the foam of the present invention, are especially advantageous in skin disorders such as psoriasis, eczema and atopic dermatitis, where the large skin areas are to be treated. The oleaginous foam compositions of the present invention provide excellent vehicles for such applications and are superior to conventional creams and ointments.

Topical Anesthetics

The compositions of the present invention may comprise a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof. Mixtures of such anesthetic agents may be synergistically beneficial.

Keratolytically Active Agents

The term “keratolytically active agent” refers herein to a compound which loosens and removes the stratum corneum of the skin, or alters the structure of the keratin layers of skin.

Keratolytically active agents are used in the treatment of many dermatological disorders, which involve dry skin, hyperkeratinization (such as psoriasis), skin itching (such as xerosis), acne and rosacea.

Suitable keratolytic agents include alpha-hydroxy acids. Alfa hydroxy acids are keratolytic, and they are also capable of trapping moisture in the skin and initiating the formation of collagen. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

Yet, another preferred keratolytic agent is urea, as well as derivatives thereof. Urea possesses both keratolytic and skin-hydration properties which are beneficial to the damaged tissue of the skin.

Another preferred group of keratolytic agents, suitable for inclusion in the therapeutic composition according to the present invention is beta-hydroxy acids, such as salicylic acid (o-hydroxybenzoic acid). Beta hydroxyl acids are keratolytic, and they are also have anti-inflammatory and antibacterial properties.

Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). In the context of the present invention, di-carboxylic acids having up to 6 carbon atoms in their skeleton are also suitable under the definition of short chain carboxylic acids having up to 6 carbon atoms in their skeleton. Non-limiting examples of suitable dicarboxylic acids are malonic acid (propanedioic acid), succinic acid (butanedioic acid), glutaric acid (Pentanedioic acid) and adipic acid (Hexanedioic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH2FCO2H), chloroethanoic acid (CH₂ClCO₂H) and dichloroethanoic acid (CHCl₂CO₂H). Dicarboxylic acids, having between about 6 and about 14 carbon atoms in their carbon atom skeleton also possess leratolytic properties. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid.

Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinol, retinal, retinoic acid, retinyl acetate, retinyl palmitate, retinyl ascorbate, isotretinoin, tazarotene, adapalene, 13-cis-retinoic acid, acitretin all-trans beta carotene, alpha carotene, lycopene, 9-cis-beta-carotene, lutein and zeaxanthin are another class of keratolytic agents, which alter the structure of the skin and promote peeling.

In certain embodiments, the keratolytic agent includes at least two keratolytic agents. At least two or more keratolytic agents in the therapeutic composition, a safe and effective peeling agent is attained, which breaks down the keratin layer of the skin, where the microorganisms reside. As a result of such breaking down of the keratin layer, the microorganisms cannot further survive in the infected area. The combination of at least two keratolytic agents enables a selective breaking down of keratin in infected skin areas, while non-infected skin areas are not affected. This phenomenon is explained by the fact that the keratin layer in infected skin areas is deformed and thus it is more vulnerable to keratolytic disintegration. Furthermore, combining at least two keratolytic agents facilitates use of each agent in a substantially minimally-irritating concentration, thus decreasing the overall irritation of the therapeutic composition.

Suitable keratolytically active agents include phenol and substituted phenolic compounds. Such compounds are known to dissolve and loosen the intracellular matrix of the hyperkeratinized tissue. As such, they are used in the treatment of dermatological disorders. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic. These compounds also exhibit antiseptic properties. Cresols also possess bactericidal and keratolytic properties.

Vitamin A and its derivatives, such as retinoic acid, isoretinoic acid, retinol and retinal are another preferred class of keratolytically active agents.

Another group of keratolytically active agents include alpha-hydroxy acids, such as lactic acid and glycolic acid and their respective salts and derivatives; and beta-hydroxy acids, such as Salicylic acid (o-hydroxybenzoic acid) and its salts and pharmaceutically acceptable derivatives, which typically possess anti-inflammatory, as well as keratolytic, activity.

Yet, another class of preferred keratolytically active agents includes urea and its derivatives.

In one or more embodiments, the keratolytic agent includes at least two keratolytic agents, from different families of chemicals. Thus, in preferred embodiments of the present invention, the keratolytic agent includes at east two agents, from different chemical families, selected from the group consisting of: (1) an alpha-hydroxy acid; (2) a beta-hydroxy acid; (3) a short-chain carboxylic acid; (4) a hydroxyl benzene; (5) a vitamin A derivative; and (6) urea. As detailed above, each of these keratolytic agent families possess, in addition to their keratolytic property, additional therapeutically-beneficial feature, such as anti-inflammatory, skin hydration and antibacterial properties for readily contributing to the overall therapeutic benefit of the therapeutic composition.

Retinoids

Another preferred group of active agents includes, for example, retinol, retinal, all trans retinoic acid and derivatives, isomers and analogs thereof, collectively termed “retinoids”. Etretinate, actiretin, isotretinoin, adapalene and tazarotene are further examples of said retinoid isomers and analogs.

In the context of the present invention, a retinoid is a compound a class of compounds consisting of four isoprenoid units joined in a head-to-tail manner, and derivatives, salts, structural analogs and functional analogs thereof, as reviewed herein in a non-limiting fashion. Typically, retinoids may be formally derived from a monocyclic parent compound containing five carbon-carbon double bonds and a functional group at the terminus of the acyclic portion.

Suitable, but non-limiting, retinoids for use in the present invention are listed below.

It is convenient to omit the explicit representation of C and H atoms in the parent skeletal structure of retinoids as follows:

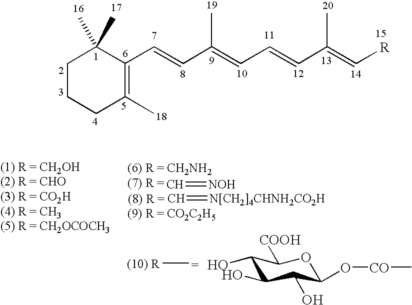

Compound (1) (2E,4E,6E,8E)-3,7-dimethyl-9-(2,6,6-trimethylcyclohex-1-en-l-yl)nona-2,4,6,8-tetraen-1-ol is also known as vitamin A, vitamin A alcohol, retinal, vitamin A₁, vitamin A₁ alcohol, axerophthol or axerol. Compound (2) also known as vitamin A aldehyde, vitamin A₁ aldehyde, retinene or retinene, and retinal or, if liable to be confused with the adjective retinal (pertaining to the retina), retinaldehyde. Compound (3) also known as tretinoin (see note), vitamin A acid or vitamin A₁ acid should be designated retinoic acid. Compound (4), is known as axerophthene. Functional substitution at the 15 position of the basic hydrocarbon is denoted by the use of the group names retinyl (R is CH₂—) or retinylidene (R is CH═), with retention of the original numbering of the basic hydrocarbon. For example (5) is retinyl acetate and (6) is retinylamine. Derivatives of retinal include for example Compound (7)—retinal oxime and Compound (8)—N⁶-retinylidene-L-lysine. Other derivatives of retinoic acid, named as carboxylic acid derivatives Compound (9)—ethyl retinoate and Compound (10)—1-O-retinoyl-b-D-glucopyranuronic acid.

Retinoids that differ in hydrogenation level from the parent structure (displayed above) are named by use of the prefixes ‘hydro’ and ‘dehydro’ together with locants specifying the carbon atoms at which hydrogen atoms have been added or removed. Examples of such retinoid compounds are Compound (11)—3,4-Didehydroretinol (also known as dehydroretinol or vitamin A₂) and Compound (12)—4,5-Didehydro-5,6-dihydroretinol (also known as alpha-vitamin A).

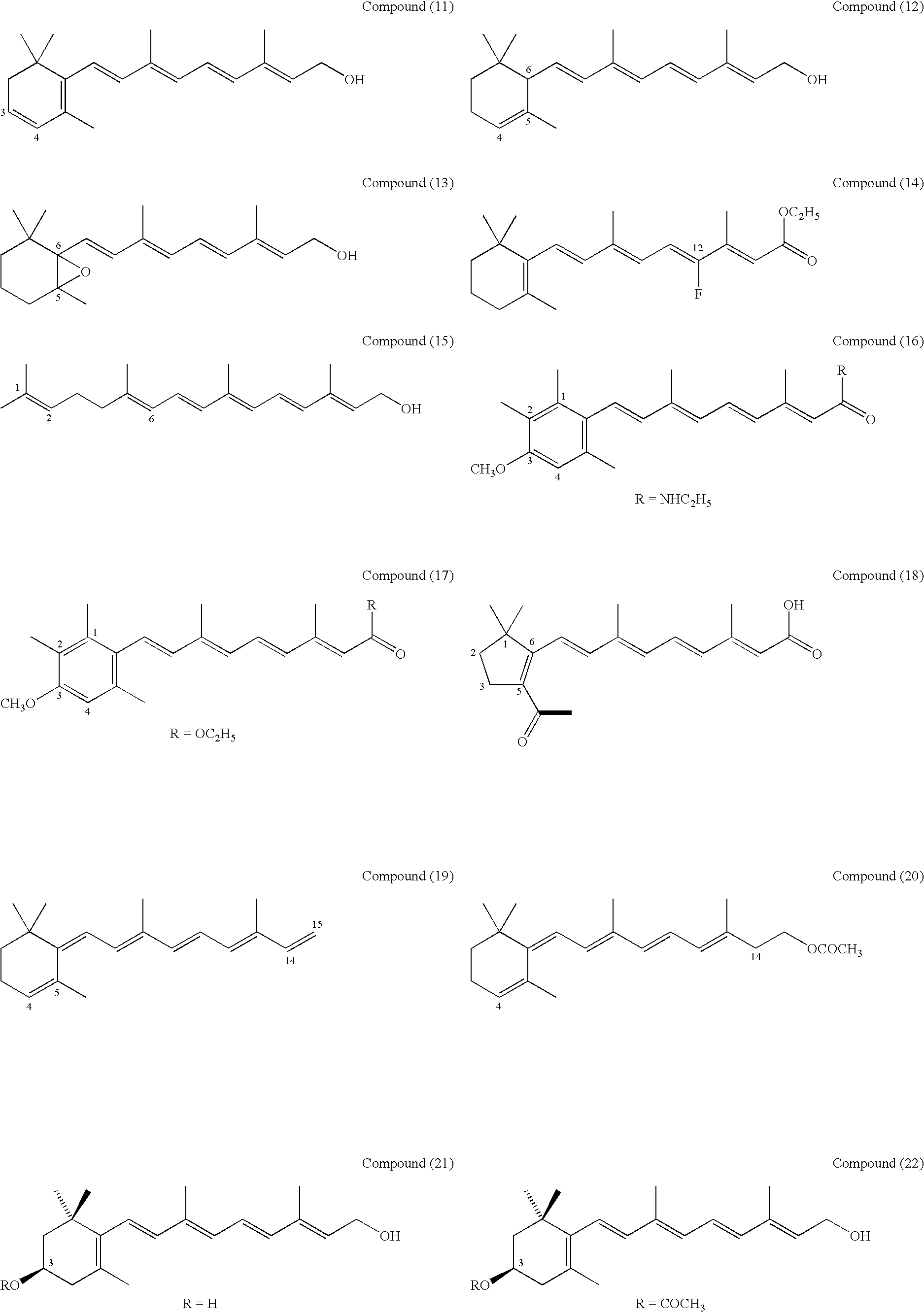

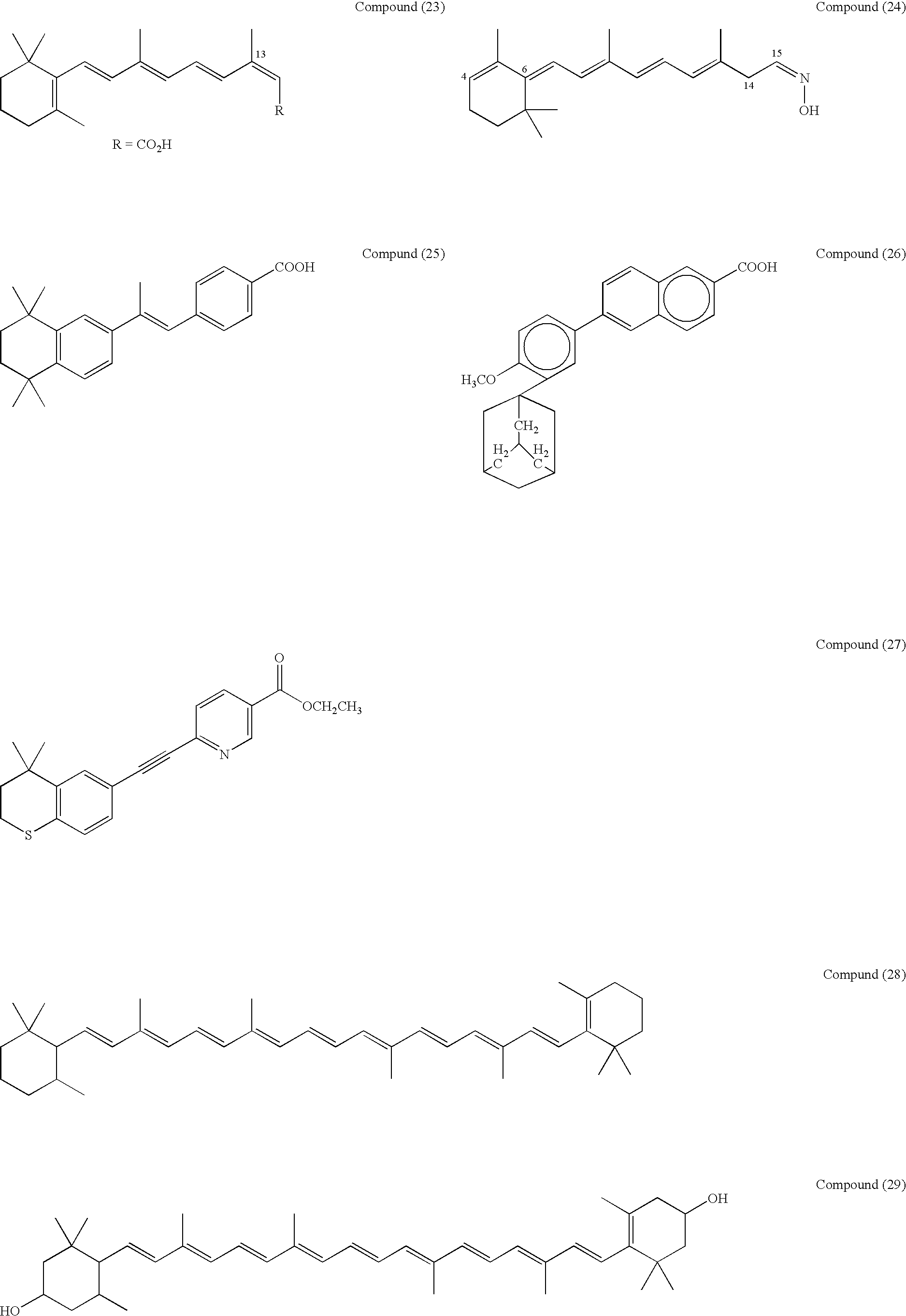

Substituted derivatives of retinoids are exemplified by Compound (13)—5,6-Epoxy-5,6-dihydroretinol (also known as hepaxanthin) and Compound (14)—Ethyl 12-fluororetinoate. Seco Retinoids are exemplified by Compound (15)—1,6-Seco-1,2-didehydroretinol, also known as g-vitamin A, and Nor Retinoids, which result from the elimination of a CH₃, CH₂, CH or C group from a retinoid are exemplified by Compound (16)—N-Ethyl-3-methoxy-2-methyl-17-nor-1,2,3,4-tetradehydroretinamide (also known as motretinide), Compound (17)—Ethyl 3-methoxy-2-methyl-17-nor-1,2,3,4-tetradehydroretinoate (also known as etretinate), acitretin (Compound (17), wherein R═H) and Compound (18)—5-Acetyl-4,18-dinor-retinoic acid. Retro Retinoids are exemplified by Compound (19)—4,5-Didehydro-15,5-retro-deoxyretinol (also known as anhydro vitamin A and Compound (20)—4,14-retro-Retinyl acetate. Stereoisomers of retinoids are exemplified by Compound (21)—(3R)-3-Hydroxyretinol and Compound (22)—(3R)-3-Acetoxyretinol. Other stereochemical isomers can are exemplified by Compound (23)—13-cis-Retinoic acid or (7E,9E,11E,13Z)-retinoic acid (also known as isotretinoin) and Compound (24)—(6E,8E,10E,12E,15Z)-4,14-retro-Retinaloxime.

‘Arotinoids or ’ retinoidal benzoic acid derivatives' contain, aromatic rings replacing either the basic β-ionone type ring structure or unsaturated bonds of the tetraene side chain of the parent retinoid skeleton, as exemplified by Compound (25) and Compound (26)—6-[3-(1-adamantyl)-4-methoxyphenyl]-2-naphthoic acid, also known as adapalene. Several artinoids, possessing potent retinoid properties, including but not limited to short retinoids, short heterocyclic retinoids, isoxazole-containing retinoids, heterocyclic isoxazole-containing retinoids, isoxazoline-containing retinoids, stilbene retinoid analogs, are disclosed in Pure Appl. Chem., Vol. 73, No. 9, pp. 1437-1444, 2001. Tazarotene (Ethyl 6-[2-(4,4-dimethylthiochroman-6-yl)ethynyl]nicotinate) is exemplary to a retinoid precursor—Compound (27), suitable as retinoid for use in the present invention.

Yet, other non-limiting exemplary retinoid precursors are carotenes, such as all-trans beta carotene—Compound (28), alpha carotene, lycopene and 9-cis-beta-carotene, as well as xanthophils (also termed “oxicarotenoids”), such as lutein and zeaxanthin—Compound (29).

Salts and derivatives of retinoid compounds are also suitable as “retinoid” for use in the present invention.

Retinoid compounds can be ascertained recognized and identified by methods known in the art. One method involves the use of competitive nuclear retinoic acid (RA and RX) receptor binding assays for identifying compounds which bind directly to the receptors. For instance, J. J. Repa et al., “All-trans-retinol is a ligand for the retinoic acid receptors”, Proc. Natl. Acad. Sci. USA, Vol. 90, pp. 7293-7297, 1993, discloses a competitive RA receptor binding assay based on human neuroblastoma cell nuclear extracts. H. Torma et al. ((1994) “Biologic activities of retinoic acid and 3,4-dehydroretinoic acid in human keratinoacytes are similar and correlate with receptor affinities and transactivation properties,” J. Invest. Dermatology, Vol. 102, pp. 49-54) discloses assays for measuring binding affinities for the nuclear retinoic acid receptors and for measuring transcriptional activation induction. M. F. Boehm et al. ((1994) “Synthesis of high specific activity [.sup.3H]-9-cis-retinoic acid and its application for identifying retinoids with unusual binding properties,” J. Med. Chem., Vol. 37, pp. 408-414) discloses a ligand-binding assay and a receptor/reporter cotransfection assay for monitor regulation of gene expression. EP 0 552 612 A2, published Jul. 28, 1993, describes ligand-binding trapping assays based on incubation of radiolabeled compounds with transfected COS-1 cells which express RA and RX receptors.

Mixtures of these retinoids may also be employed according to the present invention.

Compositions according to the present invention, which comprise retinoids as the active agent, can be used for the treatment of acne, seborrhea, various dermatoses, inflammation of the skin, mucosal membranes, vagina and the rectum, psoriasis, actinic keratosis and skin cancers, by application onto the affected area.

Calcium Channel Blockers

Calcium channel blockers are a chemically and pharmacologically heterogeneous group of drugs, but physiologically they all share the ability to selectively antagonize the calcium ion movements that are responsible for the excitation-contraction coupling in the cardiovascular system. Beyond their cardiovascular effects, calcium channel blockers are known to possess other effects, such as inhibition of the growth and proliferation of vascular smooth muscle cells and fibroblasts, inhibition of the synthesis of extracellular matrix proteins, immunomodulation, inhibition of mast cell degranulation and platelet aggregation and suppression of neutrophil adhesion and superoxide anion (0-2) production. Some calcium channel blockers also have analgesic effects.

Current therapeutic uses of calcium channel blockers include (but are not limited to) hypertension, angina, arrhythmia and subarachnoid hemorrhage. Calcium channel blockers may further relieve or prevent reactive vasodilation of migraine sufferers by inhibiting the vasoconstriction during the prodromal phase.

There are two main classes of calcium channel blockers: dihydropyridines (e.g., nifedipine, nicardipine, amlodipine, felodipine and nimodipine) and nondihydropyridines which include diltiazem (a benzothiazepine) and verapamil (a phenylalkylamine). Flunarizine is an antihistamine with calcium channel blocking activity.

In an embodiment of the present invention, the calcium channel blocker can be selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexyline, tiapamil, verapamil, pharmaceutically acceptable salts, isomers, analogs and derivatives thereof.

Cholinerqic Drugs

Cholinergic drugs produce the same effects as acetylcholine. Acetylcholine is the most common neurohormone of the parasympathetic nervous system, the part of the peripheral nervous system responsible for the every day work of the body. A cholinergic agent, also known as a parasympathomimetic agent, is a chemical which functions to enhance the effects mediated by acetylcholine in the central nervous system, the peripheral nervous system, or both. These include acetylcholine receptor agonists muscarine and nicotine, as well as anticholinesterases.

Suitable cholinergic drugs in accordance with the present invention are selected from a cholinergic agonist of acetylcholine, bethanechol, carbachol, methacholine, and pilocarpine, or an anticholinesterase of ambenonium, neostigmine, physostigmine, pyridostigmine, dyflos, and ecothinopate, and pharmaceutically acceptable salts, isomers, analogs and derivatives thereof.

Nitric Oxide Donors

Nitric oxide is an inorganic free radical, which has the chemical formula of N=O and abbreviated to NO, and is a remarkably versatile biological messenger. The chemical properties of NO are crucial in defining its biological roles, both as a transcellular signal in the cardiovascular and nervous systems and as a cytotoxic antipathogenic agent released during an inflammatory response. Endogenous NO is synthesized from the amino acid L-arginine by three isoforms of the enzyme NO synthase (NOS). The endothelial (eNOS) and neuronal (nNOS) isoforms that synthesize NO for transcellular signaling are constitutively expressed tightly regulated by a number of cofactors. These NOS isoforms typically synthesize small amounts of NO and require activation by Ca²⁺-calmodulin, making them sensitive to agents and processes that increase intracellular calcium levels. The NO generated diffuses to neighboring target cells where it acts primarily through activation of soluble guanylate cyclase (sGC) to generate cGMP from GTP, and bring about the cellular response through a reduction in intracellular calcium levels.

In an embodiment of the present invention, the nitric oxide donors can be selected from several classes, including, but not limited to inorganic nitrites and nitrates (e.g., sodium nitrite), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil, FK-409 (4-Ethyl-2-[(Z)-hydroxyiminol]-5-nitro-3(E)-hexeneamide) and derivatives thereof and hybrid NO donor drugs.

In an embodiment of the present invention, the organic nitric oxide donor includes at least one organic nitrate, which includes esters of nitric acid and may be an acyclic or cyclic compound. For instance, the organic nitrate may be ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and pharmaceutically acceptable salts, isomers, analogs and derivatives thereof.

In one embodiment of the present invention, vasoactive drugs that act via eNOS activity enhancement, such as sildenafil, vardenafil and tadalafil are also regarded “nitric oxide donors.”

Dicarboxylic acid and esters thereof.

In an embodiment of the present invention, the organic carrier comprises an ester of a dicarboxylic acid. In the context of the present invention, a dicarboxylic acid is an organic material, having two carboxylic acid moieties on its carbon atom skeleton. They have the general molecular formula HOOC—(CH₂)ₙ—COOH.

In an embodiment of the present invention, the dicarboxylic acid is a short-chain dicarboxylic acid. The simplest Short-chain dicarboxylic acid are oxalic acid (n=0), malonic acid (n=1), succinic acid (n=2) and glutaric acid (n=3).

Additional members of dicarboxylic acid group are derived from natural products or from synthesis, having “n” value from 4 up to 21. In one or more embodiments of the present invention, the dicarboxylic acid is selected from the group consisting of adipic acid (hexanedioic acid; n=4), pimelic acid (heptanedioic acid; n=5), suberic acid (octanedioic acid; n=6), azelaic acid (nonanedioic acid; n=7), sebacic acid (decanedioic acid; n=8) and dodecanedioic acid (n=10).

In an additional embodiment, the dicarboxylic acid contains 10 to 32 carbon atoms in their carbon atom skeleton, such as brassylic acid (n=11), thapsic acid (n=14), 14-methylnonacosanedioic acid (C29) and 14,15-dimethyltriacontanedioic acid (C30).

The carbon atom skeleton of the dicarboxylic acid can be saturated or unsaturated, such as in the case of maleic acid and fumaric acid.

An ester of a dicarboxylic acid is a chemical compound produced by the reaction between a dicarboxylic acid and at least one alcohol, with the elimination of a molecule of water. The reaction of a dicarboxylic acid with one alcohol molecule results in a mono ester of said dicarboxylic acid, and the reaction of a dicarboxylic acid with two alcohol molecules results in a diester of the dicarboxylic acid.

The alcohol molecule, to be linked to the dicarboxylic acid, can be selected from the group of an alkyl an aryl alcohol. Exemplary alcohol, suitable according to the present invention include methyl alcohol, ethyl alcohol, propyl alcohol, isopropyl alcohol, butyl alcohol, isobutyl alcohol, t-butyl alcohol, pentyl alcohol, hexyl alcohol, octyl alcohol, decyl alcohol, capryl alcohol, phenol, benzyl alcohol and the like.

In one or more embodiments, the alcohol is a biologically active alcohol. In an embodiment, biologically active alcohol possesses keratolytic activities. Examples of keratolytically active alcohol suitable according to the present invention include ortho-, meta- and para-hydroxyalkylbenzoate, salicylic acid, ortho-, meta-, and para-dihydroxybenzene, ortho-, meta-, and para-hydroxytoluene, alpha-hydroxy acid, retinol, and derivatives thereof such as provided in U.S. Pat. No. 6,180,669. 22. In an embodiment, the biologically active alcohol is selected from the group consisting of steroidal hormones, steroidal anti-inflammatory agents, vitamin E and vitamin D, such as provided in U.S. Pat. Appl. 20040191196.

Insecticide and Insect Repellents Agents

Insects, such as mosquitoes, biting flies, mites, gnats, fleas, chiggers, punkies, sand flies, lice and ticks can be annoying and sometimes pose a serious risk to human and animal health. In certain areas of the United States, mosquitoes can transmit diseases like equine and St. Louis encephalitis. Biting flies can inflict a painful bite that can persist for days, swell, and become infected. Ticks can transmit serious diseases like Lyme disease and Rocky Mountain spotted fever.

There are several types of insect repellents to use when protecting people and animals from flying or biting insects, spiders, ticks and mites. By way of example, these may include DEET (N,N-diethyl-m-toluamide), dimethyl phthalate, piperonyl butoxide and permethrin. Insect repelling terpenoids, have been reported by Hwang, et al, J. Chem. Ecol., 11, 1297 (1985); and Ruledge, J. Am. Mosquito Control Assoc. 4, 414 (1988).

A particularly preferred group of insect repellents includes the terpenoid compounds, described in U.S. Pat. No. 5,411,992, including:

Terpenoid-alcohol or terpene-ols are terpenoids which have at least one hydroxyl group. Examples of terpene-ols include: C10H16O compounds, perillyl alcohol, carveol, myrtenol, and cis-verbenol; C10H18O compounds, myrtanol, iso-pinocampheol, dihydrocarveol, isopulegol, terpineol, terpinen-4-ol, nerol, geraniol, and linalool, and C10H20O compounds, menthol, beta-citronellol, and dihydro-myrcenol.

Terpenoid-esters are terpenoids, which have at least one ester group which is the product of the bonding of the hydroxyl group of a terpene-ol with an aliphatic carboxylic acid that can contain functional groups such as the hydroxyl or amine on the aliphatic chain. Examples of suitable aliphatic carboxylic acids include acetic acid, propionic acid, lactic acid, and various amino acids. Examples of terpenoid-esters include: carvyl acetate, carvyl propionate, and menthyl lactate.

Essential oils which contain terpenoids and perfumes which contain terpenoids. Non-limiting examples of essential oils which have high content of terpene-ols and esters include bergamot (62% terpenoids); sage (>50% terpenoids); styrax (>50% terpenoids); peppermint (>50% terpenoids); and pine Siberian (75% terpenoids %). Terpenes, aldehydes and ketones vary in their usefulness but as a general group have potential as insect-repellent.

The oleaginous foams of the present invention are particularly suitable for the effective uniform spreading of an insect repellent agent onto large areas of the skin of humans and animals. The hydrophobic solvent present in the foam composition helps retain the insect repellent on the skin surface for an extended period of time.

Yet, in a further embodiment, the foams of the present invention are suitable for delivery of insect-killing agents (insecticides) to an afflicted external surface area of humans and animals. Thus, the pharmaceutical or cosmetic composition of the present invention may comprise an insecticide, known in the art of parasitology. By way of example, such insecticide can be selected from the group of permethrin, hexachlorobenzene, carbamate, naturally occurring pyrethroids, permethrin, allethrin, malathion, piperonyl butoxide and any combination thereof at a therapeutically effective concentration. Its application is very convenient and it spreads easily, even over hairy areas. The hydrophobic solvent present in the foam composition helps retain the insecticide on the treated area for an extended period of time. Furthermore, the presence of a hydrophobic solvent in the foam of the present invention eases mechanical removal of lice and nits with a comb.

Anti Cancer Agents

Anti cancer agents can also be used according to the present invention as the drug of choice from skin malignant tumors, such as basal cell carcinoma, squamous sell carcinoma, melanoma and Kaposi's sarcoma, as well as the pre-cancerous condition actinic keratosis. In certain cases, topical cytotoxic and antiproliferative drugs are used to treat or prevent such cancers, including 5-fluorouracil, also called 5-FU. 5-FU, as well as any other anti-cancer agents, know in the art of cancer medicine, can be incorporated in the foam at therapeutically effective levels.

A preferred family of anticancer drugs, suitable for usage in the foam of the present formulation comprises anti-estrogens, such as tamoxifen.

Photodynamic Therapy Agents

The foam compositions of the present invention are also useful to deliver photo-sensitizing agents, known in the art of photodynamic therapy. By way of example, such photosensitizers can be selected from the group comprising modified porphyrins, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives, as well as photosensitizer precursors, such as aminolevulinic acid (ALA).

Active Agents for Burns, Wounds, Cuts and Ulcers

The treatment of burns, wounds, cuts and ulcers, using the composition of the present invention is particularly advantageous. The oleaginous foam compositions of the present invention may comprise a combination of anti-infective agents (against bacteria, fungi and/or viruses), anti-inflammatory agents (steroidal and/or NSAIDs) and pain relieving components. Upon application, the foam spreads easily, covering the surface of the affected area, and without causing pain.

Cosmetic Active Agents

The oleaginous foams of the present invention are useful and advantageous for skin care and cosmetic care. The combination of oil, having refatting, protective and moisture-retaining properties, in a spreadable foam form, can be used to substitute currently used cosmetic skin care creams, lotions, gels, etc. The foam compositions of the present invention, with or without further active ingredients, are suitable for the further application as “cosmeceutical” preparation (cosmetic products with therapeutic benefit), to treat “cosmetic” skin disorders, such as aging skin, wrinkles, hyperpigmentation (melasma, chloasma, freckles, etc.), scaly skin and other skin undesirable properties.

The CTFA Cosmetic Ingredient Handbook describes a wide variety of non-limiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, astringents, etc. (e.g., clove oil, menthol, camphor, eucalyptus oil, eugenol, menthyl lactate, witch hazel distillate), anti-acne agents, anti-caking agents, antifoaming agents, anti-microbial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins and derivatives thereof.

In one embodiment the active agent is a cosmetic agent selected from a retinoid, an anti-wrinkle agent, a radical scavenger, a self-tanning agent, a skin whitening agent, a skin protective agent, an anti-cellulite agent, a massaging oil and an anti-wart agent.

Anti-Acne and Anti-Wrinkle Active Agents

The compositions of the present invention may comprise a safe and effective amount of one or more pharmaceutically or cosmetically acceptable anti-acne active agents. Examples of useful anti-acne actives include resorcinol, sulfur, salicylic acid and salicylates, alpha-hydroxy acids, nonsteroidal anti-inflammatory agents, benzoyl peroxide, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration. Certain anti-acne agents from this list are also useful in the treatment of other skin disease, such as psoriasis, eczema and atopic dermatitis.

Anti-Wrinkle Active Agents/Anti-Atrophy Active Agents and Agents to Treat Dry and Scaly Skin (xerosis and ichthyosis)

The compositions of the present invention may further comprise a safe and effective amount of one or more anti-wrinkle actives or anti-atrophy actives, which can be easily delivered by spreading a foam onto the skin. Exemplary anti-wrinkle/anti-atrophy active agents suitable for use in the compositions of the present invention include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate). In the case of dry, scaly skin (xerosis) and ichthyosis such agents can alleviate the symptoms by temporary relief of itching associated with these conditions.

Anti-Oxidants/Radical Scavengers

A safe and effective amount of an anti-oxidant/radical scavenger may be added to the compositions of the subject invention, preferably from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox.sup.R), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

The foam of the present invention is suitable for delivering skin protecting and revitalizing anti-oxidants/radical scavengers. It is further pointed out that polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA) are beneficial in the treatment of psoriasis and other skin inflammation conditions. Likewise, emollients and silicone oils exert moisture-retaining and skin protective effects on the skin. Thus in a preferred embodiment, a skin protective foam is provided, wherein the hydrophobic solvent comprises in full or in part, a solvent, selected from the group of emollients, silicone oil and oils, rich in unsaturated fatty acids, thus, affording a synergistic therapeutic effect of the anti-oxidants/radical scavenger agent and the vehicle components.

Self-Tanning Active Agents

The oleaginous foams of the present invention are particularly suitable for the uniform delivery of a tanning active agent onto large areas of the skin. It is preferable that the compositions comprise from about 0.1% to about 20%, more preferably from about 2% to about 7%, and still more preferably from about 3% to about 6% of the composition, of dihydroxyacetone, or any other compound, know in the art as an artificial tanning active agent.

Solid Matter Agents

According to a preferred embodiment of the present invention, the at least one active agent comprises solid matter or particulate matter i.e., material that is not soluble in the liquid carrier composition of the foamable composition. For definition purposes, solid matter shall mean material that is not soluble in the foamable composition more than 10% of the concentration intended to be included in said foamable composition. The concentration of the solid matter in the foamable composition is from about 1% to about 20% w/w. In one or more embodiments, the concentration of solid matter in the composition is from about 2% to about 16% w/w.

By way of example, the following classes of solid matter substances are presented.

Metallic oxides, such as titanium dioxide, zinc oxide, zirconium oxide, iron oxide. Preferably, as used in the present invention, titanium dioxide has an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. In one embodiment the metal oxides are present in the amount of from about 0.1% to about 20%, preferably from about 0.5% to about 16%, more preferably from about 1% to about 10%, of the composition. In yet another embodiment, such solids are micronized to form particles having primary size of less than 15 nm.

Silicon containing solid matter includes silicone oxide, also termed “silica”, “fumed silica” and “silica gel”, a white or colorless insoluble solid (SiO2); and talc, which is fine grained mineral consisting of hydrated magnesium silicate;

Carbon, for example in the form of amorphous carbon or graphite;

Oxidizing agents, such as benzoyl peroxide, calcium and magnesium hypochlorite;

Metallic Silver, in small particles, including nanocrystalline silver, which is used for antibacterial and wound healing purposes; other metal particles and mineral particles

Cosmetic scrub materials, including, for example meals of strawberry seeds, raspberry seeds, apricot seeds, sweet almond, cranberry seeds;

Pigments, which are insoluble in the foamable composition.

When such solid matter agents are included in the oleaginous foamable composition of the present invention, a novel foam product, combining the refatting, occlusive and protective properties of the oleaginous foam carrier and the beneficial properties of the solid matter agent is afforded. Thus, several unique products can be provided, as exemplified herein:

Generally, products for the prevention and treatment of diaper dermatitis and for skin protection are provided in the form of paste that is intended for application on the baby's posterior, under the diaper. The paste typically includes about 30% oil and/or petrolatum, and about 10% zinc oxide, which are intended to provide a protective barrier between the baby's skin and the irritating environment inside the diaper. While containing the right ingredients, current baby pastes are very viscous and thick, and therefore hard to spread on the target area.

The oleaginous foam for treating or preventing diaper rash of the present invention comprises the following ingredients:

at least one solvent selected from a hydrophobic solvent, a co-solvent, an emollient and mixtures thereof, at a concentration of about 30% to about 90%, preferably between about 30% to about 70%

water at a concentration of 1% to about 60%;

about 6% to about 20% zinc oxide (or an alternative metal oxide)

at least one non-ionic lipophilic surface active agent, preferably having an HLB value of about 3 to about 10, more preferably about 3.5 to about 9 at a concentration of about 0.1% to about 10%, or between about 0.1% and about 5%;

at least one gelling agent at a concentration of about 0.1% to about 5%;

a liquefied or compressed gas propellant at a concentration of about 3% to about 25% of the total composition, in an aerosol container.

Such foam is superior to current pastes in that it is very fluffy and light. Upon discharge from the aerosol can, it creates a mass, having density between 0.04 g/mL and 0.2 g/mL, which is very easy to spread evenly and uniformly on the target area. There is no need to rub thoroughly and therefore, application of the foam does not cause any discomfort to the baby, unlike conventional baby pastes. Following application and spreading of the foam, a protective layer is formed, which is water resistant, and does not wash out under a stream of tap water.

Foam for diaper dermatitis and/or skin protection can further comprise anti-irritant and/or infective agents, such as corticosteroids, anti-inflammatory, anti-allergic, anti-fungal and anti-microbial agents.

Skin-Lightening and Whitening Agents

The foam of the present invention is particularly suitable for the uniform delivery of a skin-lightening agent. When used, the compositions preferably comprise from about 0.1% to about 10%, more preferably from about 0.2% to about 5%, of the composition, of a skin-lightening agent. Suitable skin lightening or whitening agents include those known in the art, including hydroquinone, azelaic acid and other related dicarboxylic acids, and salts and derivatives thereof, retinoids, kojic acid, arbutin, nicotinic acid and its precursors, salts and derivatives, ascorbic acid and salts and derivatives thereof (e.g., magnesium ascorbyl phosphate or sodium ascorbyl phosphate), and herbal extracts (e.g., mulberry extract, placental extract).

In one or more embodiments of the present invention, the foam composition comprises a combination of a skin-whitening agent and a sunscreen agent.

In one or more embodiments of the present invention, the foam composition comprises a combination of a skin-whitening agent and an inorganic sunscreen agent. When inorganic sunscreen agents, e.g. Ti02, are rubbed onto the skin, they leave a white coating, which provides an immediate (although transient) whitening effect, which is highly desirable by the consumer, who wishes to see instant change in his/her appearance. The whitening agent, in combination with the inorganic sunscreen agent in the foam carrier can be easily and uniformly distributed on the skin surface, thereby affording an even instant whitening effect, unlike creams that are difficult to spread evenly on skin areas.

Use of a Solvent, Surface Active Agent, Foam Adjuvant and Polymeric Agent as an Active Agent.

According to one embodiment, the at least one active agent is selected from the group of solvent, surface active agent, foam adjuvant and gelling agent, which are, on a case by case basis known to possess a therapeutic benefit.

Composition and Foam Physical Characteristics

Composition Flow Properties

It is desirable to have an oleaginous foam composition, including solvents, formulation excipients, water (as applicable), active agents and propellant, in a stable formulation, which provides acceptable shelf-life of the product.

Yet, another crucial property of a composition is its level of flow, since a composition that is not free flowing cannot flow through the dip-tube of the aerosol container and create acceptable foam. It has been noted that in the context of the composition of the present invention, compositions comprising semi-solid hydrophobic solvents, e.g., white petrolatum, are excessively viscous and demonstrate poor flowability.

The combination of at least one surface active agent, at least one foaming adjuvant and at least one gelling agent, according to one or more embodiments of the invention provides a low specific gravity foam having superior expandability, flow properties and sheer breakability (among other attributes). According to one or more embodiments of the present invention, the total amount of at least one surface active agent, at least one foam adjuvant (optional) and at least gelling agent, in combination does not exceed 8% (w/w) of foamable composition. In other embodiments, the combined amounts of at least one surface active agent, at least one foaming adjuvant and at least one gelling agent is less than 5% (w/w) of foam composition. The low solid content improves the flow properties of the foam, reduces unpleasant skin residue and reduces the cost of manufacture. As is demonstrated herein, the foam stability and expandability are excellent, despite the low levels of these components in the foam.

Expandability

Expandability is an important feature of a product, intended to treat large surface areas and internal cavities of the body. Thus, in one embodiment of the present invention, the specific gravity of the foam, upon discharge from the aerosol can is between about 0.02 g/mL and 0.5 g/mL, more preferably between about 0.04 g/mL and about 0.2 g/mL.

Foam Physical Characteristics

In terms of foam consistency and texture an acceptable foam is one, that exhibits the following characteristics:

Upon release from the aerosol can, creates a foam mass, which is sustained on a surface for at least one minute;

Foam texture should vary from a very fine creamy foam to a fine bubble structure;

Foam has to have specific gravity in the range of about 0.02 g/mL to about 0.5 g/mL, more preferably between about 0.04 g/mL and about 0.2 g/mL.

In terms of spreadability and absorption an acceptable foam is one, that:

Does not readily collapse upon dispensing on the skin;

Spreads easily on a skin surface;

Substantially absorbed following rubbing onto the skin.

In terms of organoleptic properties an acceptable foam is one, that:

Creates a pleasant feeling after application;

Leaves minimal oily residue;

Leaves minimal shiny residual look.

The following scale for foam quality is used to evaluate foams.

E (excellent): very rich and creamy in appearance, does not show any bubble structure or shows a very fine (small) bubble structure.

G (good): rich and creamy in appearance, very small bubble size, “dulls” more rapidly than an excellent foam.

FG (fairly good): a moderate amount of creaminess noticeable, bubble structure is noticeable.

F (fair): very little creaminess noticeable, larger bubble structure than a “fairly good” foam.

P (poor): no creaminess noticeable, large bubble structure.

VP (very poor): dry foam, large very dull bubbles, difficult to spread on the skin.

Foam Stability and Breakability

In one or more embodiments, the foam compositions are desirably stable for a long period of time. Thus, the foam composition does not undergo phase separation following at least two freeze/thaw cycles.

According to further embodiments, upon discharge from an aerosol can onto a mucosal membrane at about 37° C., the foam expands to reach its designated volume and stays stable as a foam for at least 60 seconds following application, or about 2 minutes, or even about 3 minutes.

A crucial aspect of foam properties, according to the present invention is breakability. Sheer-force breakability of the foam, as attained by the composition of the present invention is clearly advantageous to thermally-induced breakability, present, for example in U.S. Pat. No. 6,126,920, and the respective Olux® and Luxiq® products, as demonstrated by the fact that according to the use instructions of Olux® and Luxiq®, the foam cannot be applied on the hand and afterwards delivered to the afflicted area, since it collapses upon exposure to skin temperature.

Further Technical Parameters

The composition of the present invention may be contained in and dispensed from a container capable of withstanding the pressure of the propellant gas and having an appropriate valve/nozzle for dispensing the composition as foam under pressure. A customary liquefied or compressed gas propellant can be added, in the amount of about 3 to about 25% of the total composition. Liquefied propellants are gases that exist as liquids under pressure, including high purity hydrocarbons such as propane, isobutane and n-butane, dimethyl ether and chlorofluorocarbons (CFCs). Compressed gasses are exemplified by air, nitrogen and carbon dioxide.

A specific embodiment according to the present invention comprises placing the composition of the present invention on a patch, occlusive tape or the skin-contact compartment of a transdermal delivery apparatus and applying such object onto the skin, in order to attain effective superficial treatment or enhanced penetration of the drug into the skin or through the skin.

Utilizing such strategy, one can apply drugs, which are currently administered systemically or that require transdermal delivery, in the preferred therapeutic system of the present invention. Examples for such drugs are nicotine, testosterone and other male hormones and male hormone precursors, estrogen and other female hormones and hormone precursors, growth hormone, insulin, caffeine, steroidal and non-steroidal antiinflammatory agents and thyroid hormone substitutes.

The therapeutic composition according to the present invention can also be used to prepare cosmetics for beauty purpose by adding into skin care agents and perfume.

Metered Dosing

In order to provide proper therapy, precise dosing is advantageous. According to one preferred embodiment, the foam therapeutic product is adapted for storage in an aerosol container having a metered dose valve associated therewith for dispensing an accurate dose of drug in the form of a foam. More preferably, the metered dose valve is selected to release a foam in a volume that will allow effective spreading of the active agent throughout the body surface with substantially minimal overdose.

In one or more embodiments, the meter dose valve provides a unit dose of between about 10 μL and about 1000 μL. Assuming a representative foam density (specific gravity) of 0.06 g/mL, a 10 μL valve provides a volume of about 0.17 mL of foam, and a 1000 μL metered dose valve provides about 17 mL of foam. Thus, by selecting a specific metered dosing valve and adjusting the foam density by fine tuning formulation parameters and adjusting the ration between the liquid components of the composition and the propellant, one can design an adequate dosage form according to the specific target body surface.

Fields of Pharmaceutical Applications

By including an appropriate therapeutic agent in the foamable carrier, the foam composition of the present invention is useful in treating a patient having a any one of a variety of dermatological disorders (also termed “dermatoses”), such as classified, in a non-limiting exemplary manner, according to the following groups: Dermatitis including Contact Dermatitis, Atopic Dermatitis, Seborrheic Dermatitis, Nummular Dermatitis, Chronic Dermatitis of the hands and feet, Generalized Exfoliative Dermatitis, Stasis Dermatitis; Lichen Simplex Chronicus; Diaper rash; Bacterial Infections including Cellulitis, Acute Lymphangitis, Lymphadenitis, Erysipelas, Cutaneous Abscesses, Necrotizing Subcutaneous Infections, Staphylococcal Scalded Skin Syndrome, Folliculitis, Furuncles, Hidradenitis Suppurativa, Carbuncles, Paronychial Infections, Erythrasma; Fungal Infections including Dermatophyte Infections, Yeast Infections; Parasitic Infections including Scabies, Pediculosis, Creeping Eruption; Viral Infections; Disorders of Hair Follicles and Sebaceous Glands including Acne, Rosacea, Perioral Dermatitis, Hypertrichosis (Hirsutism), Alopecia, including male pattern baldness, alopecia areata, alopecia universalis and alopecia totalis; Pseudofolliculitis Barbae, Keratinous Cyst; Scaling Papular Diseases including Psoriasis, Pityriasis Rosea, Lichen Planus, Pityriasis Rubra Pilaris; Benign Tumors including Moles, Dysplastic Nevi, Skin Tags, Lipomas, Angiomas, Pyogenic Granuloma, Seborrheic Keratoses, Dermatofibroma, Keratoacanthoma, Keloid; Malignant Tumors including Basal Cell Carcinoma, Squamous Cell Carcinoma, Malignant Melanoma, Paget's Disease of the Nipples, Kaposi's Sarcoma; Reactions to Sunlight including Sunburn, Chronic Effects of Sunlight, Photosensitivity; Bullous Diseases including Pemphigus, Bullous Pemphigoid, Dermatitis Herpetiformis, Linear Immunoglobulin A Disease; Pigmentation Disorders including Hypopigmentation such as Vitiligo, Albinism and Postinflammatory hypopigmentation and Hyperpigmentation such as Melasma (chloasma), Drug-induced hyperpigmentation, Postinflammatory hyperpigmentation; Disorders of Cornification including Ichthyosis, Keratosis Pilaris, Calluses and Corns, Actinic keratosis; Pressure Sores; Disorders of Sweating; Inflammatory reactions including Drug Eruptions, Toxic Epidermal Necrolysis; Erythema Multiforme, Erythema Nodosum, Granuloma Annulare.

The oleaginous compositions of the present invention are useful in the therapy of non-dermatological disorders, which respond to topical/transdermal delivery of an active agent. By way of example, such disorders include localized pain in general, as well as joint pain, muscle pain, back pain, rheumatic pain, arthritis, ostheoarthritis and acute soft tissue injuries and sports injuries. Other disorders of this class include conditions, which respond to hormone therapy, such as hormone replacement therapy, transdermal nicotine administration, and other respective disorders, known in the art of drug delivery.

The oleaginous compositions of the present invention are further useful for the treatment and prevention of disorders and diseases of other body cavities including the rectum, vagina, penile urethra and ear canal.

Thus, the oleaginous foam compositions of the present invention are useful in treating a patient having any one of a variety of gynecological disorders, such as classified, in a non-limiting exemplary manner, according to the following groups:

Pelvic pain, including premenstrual syndrome (PMS), mittelschmerz (severe midcycle pain due to ovulation), dysmenorrhea (pain related to the menstrual cycle), endometriosis, ectopic pregnancy, ovarian cysts and masses, acute pelvic inflammatory disease, pelvic congestion syndrome and vulvodynia; vulvovaginal infections, including bacterial vaginosis, candidal vaginitis, trichomonas vaginalis, herpes simplex genital ulcers and warts, pelvic inflammatory disease (PID), cervicitis, acute and chronic salpingitis; endometriosis; gynecological neoplasms, including endometrial Cancer, ovarian cancer, cervical cancer, vulvar cancer, vaginal cancer, fallopian tube cancer and gestational trophoblastic disease; benign tumors; sexually transmitted diseases; sexual dysfunction disorders that respond to pharmacological therapy, including sexual arousal disorder, female orgasmic disorder, dyspareunia and vaginismus; and various gynecological disorders that respond to hormonal therapy.

The foam according to one or more embodiments of the present invention can be used as a lubricating foam. Without limitation, the lubricating foam is useful in lubrication of the birth canal for easy passage of a newborn baby or the vaginal cavity during intercourse.

Rectal applications include, for example, anal abscess/fistula, anal cancer, anal warts, Crohn's disease, haemorrhoids, anal and perianal pruritus, soreness, and excoriation, perianal thrush, anal fissures, fecal incontinence, constipation, polyps of the colon and rectum.

The oleaginous foam compositions of the present invention are further useful for intra-vaginal and rectal treatment of sexually-transmitted and non-sexually-transmitted infectious disease (STDs).

In one or more embodiments, the invention provides a method of treatment of a disorder of the skin, mucosal membrane, ear channel, vaginal, rectal and penile urethra disorders, comprising topical application of the foam composition of the present invention, whereby one or more active agents, in a therapeutically effective concentration to the afflicted area.

In a further embodiment, the invention provides a method of treatment of a non-dermatological disorder, which responds to topical delivery of an active agent, comprising topical application of the foam composition of the present invention, whereby one or more active agents, in a therapeutically effective concentration to the skin.

Treatment/Therapy

The terms “therapy” and “treatment” as used herein interchangeably, cover any treatment of a disease or disorder, and includes, for example:

(i) Curing the disease or disorder;

(ii) preventing the disease or disorder from occurring in a subject which may be predisposed to the disease but has not yet been diagnosed as having it;

(iii) inhibiting the disease or disorder;

(iv) relieving the disease or disorder;

(v) causing regression of the disease;

(vi) providing a beneficial immunological effect;

(vii) improving the quality of life of a subject afflicted by a disease or disorder; and, in the case of cosmetic treatment (viii) cleansing, beautifying, promoting attractiveness, or altering the appearance without affecting the body's structure or functions In the following, some non-limiting examples and experiments are described in detail. This invention is not limited to these examples and experiments. Many variations will suggest themselves are within the full intended scope of the appended claims.

The waterless formulas of the present invention may be made in the following general methodology set out below with appropriate adjustments for each formulation as will be appreciated by someone skilled in the art. Polymers are mixed, swelled and solubilized in the main solvent, when necessary, with appropriate heating to 70 C or cooling as appropriate for specific polymer until it forms a clear solution. Stabilizing surfactants added usually with heat, until a homogeneous mixture is obtained, the mixture is then allowed to cool to below 40 C. The remainder of the ingredients are then added with mixing until they have dissolved in the medium. The active agent is usually added at the end. Cool to room temperature. The canisters are then filled with the above waterless formula, sealed and crimped with a valve and pressurized with the propellant.

For emulsion formulas:
1. Mix oily phase ingredients and heat to 75 C to melt all ingredients and obtain homogeneous mixture.
2. Mix polymers in water with heating or cooling as appropriate for specific polymer.
3. Add all other water soluble ingredients to water-polymer solution and heat to 75 C.
4. Add slowly internal phase to external phase at 75 C under vigorous mixing and homogenize to obtain fine emulsion.
5. Cool to below 40 C and add sensitive ingredients with mild mixing.
6. Cool to room temperature

For Oily Waterless Foam:
1. Mix all ingredients excluding polymers and heat to 75 C to melt and dissolve and obtain homogeneous mixture.
2. Mix well and cool to below 40 C and add the polymers and sensitive ingredients with moderate mixing.
3. Cool to room temperature

### EXAMPLE 1

### Anhydrous Foam Comprising a Potent Solvent and MCT Oil

The components of the anhydrous foam are listed in the table below.

| Ingredient | Synonym | Function | % | % | % | % | % |
|---|---|---|---|---|---|---|---|
| n-Methyl | NMP | Potent solvent | 68.4 | 0 | 0 | 0 | 0 |
| pyrrolidone | | | | | | | |
| Propylene glycol | | Potent solvent | 0 | 69.5 | 0 | 0 | 0 |
| Glycofurol | | Potent solvent | 0 | 0 | 69.5 | | 69.5 |
| Dimethyl | Arlasolve | Potent solvent | 0 | 0 | 0 | 70.0 | 0 |
| isosorbide | | | | | | | |
| MCT oil | Caprylic/Capric | hydrophobic | 9.0 | 9.0 | 9.0 | 9.0 | 9.0 |
| | Triglycerides | solvent | | | | | |
| Hexylene glycol | | Co-solvent | 2.1 | 2.1 | 2.1 | 2.1 | 2.1 |
| Glyceryl | | Stabilizer | 1.8 | 1.8 | 1.8 | 1.8 | 1.8 |
| monostearate | | | | | | | |
| Stearyl alcohol | | Stabilizer | 1.8 | 1.8 | 1.8 | 1.8 | 1.8 |
| Oleylalcohol | | Foam adjuvant | 2.3 | 2.3 | 2.3 | 2.3 | 2.3 |
| Sisterna SP-30 | Sucrose ester | Surfactant | 0.9 | 0.9 | 0.9 | 0.9 | 0.9 |
| Sisterna SP70 | Sucrose ester | Surfactant | 0.9 | 0.9 | 0.9 | 0.9 | 0.9 |
| Klucel MF | Hydroxypropyl | Gelling agent | 0.4 | 0.4 | 0.4 | 0.4 | 0.4 |
| | methylcellulose | | | | | | |
| Phenonip | Methyl, butyl, | Preservative | 0.3 | 0.3 | 0.3 | 0.3 | 0.3 |
| | propyl paraben, | | | | | | |
| | phenoxyethanol | | | | | | |
| Betamethasone | | Active agent | 0.1 | 0 | 0 | 0 | 0 |
| valerate | | | | | | | |
| Mupirocin | | Active agent | 0 | 1.0 | 0 | 0 | 0 |
| Ketoconazole | | Active agent | 0 | 0 | 1.0 | 0 | 0 |
| Cyclosporine | | Active agent | 0 | 0 | 0 | 0.5 | 0 |
| Acyclovir | | Active agent | 0 | 0 | 0 | 0 | 5 |
| Propane/butane | | Propellant | 12.0 | 10.0 | 10.0 | 10.0 | 10.0 |

Notes:
The liquefied or gas propellant can be added at a concentration of about 3% to about 25%.
The compositions used only non-ionic surface active agents, in a concentration of about 2%, and the total amount of surface active agent, foam adjuvants and polymeric agent ranged from about 4% to about 6% (w/w).
The foam of this example having a density of about 0.2 g/mL is useful as a carrier of additional active agents. It is also useful as lubricating foam, for various purposes.

### EXAMPLE 2

The components of the oil/glycerin foam are listed in the table below.

| Ingredient | Synonym | Function | % | % | % | % | % | % |
|---|---|---|---|---|---|---|---|---|
| Caprylic/Capric | MCT oil | hydrophobic | 60.9 | 60.0 | 59.0 | 60.0 | 60.0 | 56.0 |
| Triglycerides | | solvent/potent | | | | | | |
| | | solvent | | | | | | |
| Propylene glycol | | Co-solvent/potent | 10.0 | 10.0 | | | 5.0 | 5.0 |
| | | solvent | | | | | | |
| Hexylene glycol | | Co-solvent/potent | | | 10.0 | 5.0 | | |
| | | solvent | | | | | | |
| Purified water | De-ionized | Solvent | 10.0 | 10.0 | 10.0 | 10.0 | 10.0 | 10.0 |
| | Distilled water | | | | | | | |
| Potent solvent | | | — | — | — | 5.0 | 5.0 | 5.0 |
| Lecithin | Phospholipids | Surfactant | 10.0 | 10.0 | 10.0 | 10.0 | 10.0 | 10.0 |
| Stearyl alcohol | Stearyl alcohol | Stabilizer | 5.0 | 5.0 | 5.0 | 5.0 | 5.0 | 5.0 |
| Glyceryl | Glyceryl | Stabilizer | 2.0 | 2.0 | 2.0 | 2.0 | 2.0 | 2.0 |
| monostearate | monostearate | | | | | | | |
| PVP K90 | Polyvinyl | Gelling agent | 2.0 | 2.0 | 2.0 | 2.0 | 2.0 | 2.0 |
| | pyrrolidone | | | | | | | |
| Preservative | | | 0.3 | 0.3 | 0.3 | 0.3 | 0.3 | 0.3 |
| Betamethasone | | Active agent | 0.1 | | | | | |
| valerate | | | | | | | | |
| Mupirocin | | Active agent | | 1.0 | | | 1.0 | |
| Ketoconazole | | Active agent | | | 2.0 | | | |
| Tacrolimus | | Active agent | | | | 1.0 | | |
| Acyclovir | | Active agent | | | | | | 5.0 |
| Propane/butane | | Propellant | 12.0 | 10.0 | 10.0 | 10.0 | 10.0 | 10.0 |

Notes:
The liquefied or gas propellant can be added at a concentration of about 3% to about 25%.
The potent solvent and hexylene glycol (emollient) may be optionally incorporated.
In these particular examples, the water content was minimal and necessary for the gelling agent incorporation; higher levels of water are an option.
Lecithin is provided as the surfactant. Several types of powdered, de-oiled and liquid (55% to 80% Phosphatidyl choline) phospholids have been tested successfully for the production of acceptable foams.
In certain examples, polyvinylpyrrolidone (PVP) was shown to be the preferred gelling agent.
The compositions use only non-ionic surface active agents, in concentration of about 2%, and the total amount of surface active agent, foam adjuvants and polymeric agent ranged from about 4% to about 6% (w/w).
The foam of this example is useful as a carrier of additional active agents. It is also useful as lubricating foam, for various purposes.
Stearyl alcohol, cetyl alcohol or oleyl alcohol (foam adjuvants) and co-solvents, such as propylene glycol and hexylene glycol, are optionally incorporated in the foam.
Density of the foam is about 0.08 to about 0.12 g/mL

### EXAMPLE 3

### Oil/Glycerin Foam

The components of the oil/glycerin foam are listed in the table below.

| Ingredient | Synonym | Function | % | % | % | % |
|---|---|---|---|---|---|---|
| Glycerin | Glycerol | Co-solvent | 32.0 | 32.0 | 32.5 | 40.5 |
| Purified water | | Solvent | 17.0 | 17.0 | 18.55 | 14.05 |
| MCT oil | Caprylic/Capric | Hydrophobic | 9.0 | 9.0 | 9.0 | 8.0 |
| | Triglycerides | Solvent | | | | |
| Isopropyl myristate | IPM | Co-solvent | 0 | 0 | 9.0 | 8.0 |
| Isopropyl palmitate | IPP | Co-solvent | 0 | 10.0 | 0 | 0 |
| Diisopropyl adipate | DISPA | Co-solvent | 9.0 | 0 | 0 | 0 |
| Hexylene glycol | Hexylene glycol | Emollient | 9.0 | 9.0 | 9.0 | 8.0 |
| Oleyl alcohol | Oleyl alcohol | Foam adjuvant | 9.0 | 9.0 | 9.0 | 8.0 |
| Sisterna sp-50 | Sucrose ester | Surfactant | 1.8 | 1.8 | 1.8 | 1.8 |
| Glyceryl | Glyceryl | Stabilizer | 0.4 | 0.4 | 0.4 | 0.4 |
| monostearate | monostearate | | | | | |
| Pemulen TR2 | Acrylates/C10-30 | Stabilizer | 0.1 | 0.1 | 0.1 | 0.1 |
| | Alkyl Acrylate | | | | | |
| | Cross-Polymer | | | | | |
| Methocel K100M | Methyl cellulose | Gelling agent | 0.3 | 0.3 | 0.3 | 0.3 |
| TEA | Tri-ethanolamine | Neutralizer | 0.05 | 0.05 | 0.05 | 0.05 |
| Phenonip | Methyl, butyl, | Preservative | 0.25 | 0.35 | 0.3 | 0.3 |
| | propyl paraben, | | | | | |
| | phenoxyethanol | | | | | |
| Betamethasone | | Active agent | 0.1 | 0 | 0 | 0 |
| valerate | | | | | | |
| Mupirocin | | Active agent | 0 | 1.0 | 0 | 0 |
| Ketoconazole | | Active agent | 0 | 0 | 2.0 | 0 |
| Cyclosporine | | Active agent | 0 | 0 | 0 | 0.5 |
| Propane/butane | | Propellant | 12.0 | 10.0 | 8.0 | 10.0 |

Notes:
The liquefied or gas propellant can be added at a concentration of about 3% to about 25%.
In non-limiting examples, the oil/glycerin foams of the present invention comprise about 10% to about 20% water, about 37% glycerin and about 30% oil blend and about 10% hexylene glycol.
The compositions use only non-ionic surface active agents, in concentration of about 2%, and the total amount of surface active agent, foam adjuvants and polymeric agent ranged from about 8% to about 12% (w/w).
The foam of this example is useful as a carrier of additional active agents. It is also useful as lubricating foam, for various purposes.
Density of the foam is about 0.18 g/mL to about 0.20 g/mL.
Upon release from the aerosol can, foam is released, and stays stable for several minutes, until it is rubbed onto the afflicted area, then it is immediately broken down and absorbed. This property enables convenient and even application with good sensory feeling.

### EXAMPLE 4

### Compositions Comprising PEG

Compositions comprising polyethylene glycol (PEG) derivatives have been prepared and shown to be excellent foams. According to the following non-limiting example the composition comprises about 80% to about 97.5% PEG 400, about 1% to about 5% of at least one surface active agent having HLB between 2 and 9 and 0.5% gelling agent, prior to the addition of a propellant (about 10% of the total composition). Notably the following compositions did not comprise any water at all.

PEG 400 Foamable Compositions (Vehicle)

| | % w/w | % w/w | % w/w | % w/w | % w/w | % w/w | % w/w |
|---|---|---|---|---|---|---|---|
| PEG400 | 87.50 | 91.50 | 87.50 | 89.50 | 87.50 | 87.50 | 87.50 |
| Klucel MX (hydroxypropyl | 0.50 | 0 | 0.50 | 0 | 0.50 | 0 | 0.50 |
| cellulose) | | | | | | | |
| Klucel LF (hydroxypropyl | 0 | 0.50 | 0 | 0.50 | 0 | 0.50 | 0 |
| cellulose) | | | | | | | |
| Lipocol C2 (POE (2) cetyl | 2.00 | 2.00 | 0 | 0 | 0 | 0 | 0 |
| ether) | | | | | | | |
| Myrj 52 | 0 | 0 | 2.00 | 2.00 | 0 | 0 | 0 |
| Steareth-2 | 0 | 0 | 0 | 0 | 2.00 | 2.00 | 0 |
| Dermofeel G10L (Polyglyceryl- | 0 | 0 | 0 | 0 | 0 | 0 | 2.00 |
| 10 | | | | | | | |
| Laurate) | | | | | | | |
| Propellant | 10 | 6 | 10 | 8 | 10 | 10 | 10 |
| Density | 0.060 | 0.063 | 0.063 | 0.055 | 0.052 | 0.050 | 0.075 |

Notes:
The liquefied or gas propellant can be added at a concentration of about 3% to about 25%.
The foams of this example have a non-ionic surface active agent at a concentration of 2%. Total amounts of surface active agent foam adjuvant and polymeric agent is in the range of 2.5%.

The compositions are useful as carriers of various active therapeutic active agents.

The following table exemplifies the use of PEG 400 as a potent solvent for Mupirocin, which is practically insoluble in mineral oil and other commonly used ointment solvents. Note that Mupirocin is incompatible with most solvents and thus, a foam comprising PEG 400 as the sole solvent is highly valuable.

PEG 400 Foamable Compositions. Comprising Mupirocin

| | % w/w | % w/w | % w/w |
|---|---|---|---|
| Mupirocin | 2.00 | 2.00 | 2.00 |
| PEG400 | 89.50 | 89.50 | 89.50 |
| Klucel LF (hydroxypropyl | 0.50 | 0.50 | 0.50 |
| cellulose) | | | |
| Steareth-2 | 2.00 | 1.00 | 0 |
| Polyglyceryl-10 | | | 2.00 |
| Laurate | | | |
| Propellant (Propane/butane)* | 6.0 | 6.0 | 6.0 |
| Density | 0.060 | 0.060 | 0.062 |

Notes:
*The liquefied or gas propellant can be added at a concentration of about 3% to about 25%.
**The foams of this example have a non-ionic surface active agent at a concentration of 2%. Total amounts of surface active agent foam adjuvant and polymeric agent is in the range of 2.5% (w/w).

### EXAMPLE 5

### Hydrophilic Peg Containing Compositions with Various Active Agents

Stock Solution:

| Ingredient name | % w/w |
|---|---|
| PEG-400 | 97.50 |
| Hydroxypropyl cellulose | 0.50 |
| Steareth 2 | 2.00 |
| Total | 100.00 |

A

| | | | | | | | | |
|---|---|---|---|---|---|---|---|---|
| PEG 400 | 95.00 | 85.00 | 95.00 | 99.88 | 95.00 | 99.995 | 98.00 | 98.00 |
| Hydroxypropyl | | | | | | | | |
| cellulose | | | | | | | | |
| Steareth 2 | | | | | | | | |
| Acyclovir | 5.00 | | | | | | | |
| Azelaic acid | | 15.00 | | | | | | |
| Benzoyl peroxide | | | 5.00 | | | | | |
| Betamethasone | | | | 0.12 | | | | |
| 17 valerate | | | | | | | | |
| micronized | | | | | | | | |
| Caffeine | | | | | 5.00 | | | |
| Calcipotriol | | | | | | 0.005 | | |
| hydrate | | | | | | | | |
| Ciclopiroxolamine | | | | | | | 2.00 | |
| Diclofenac sodium | | | | | | | | 1.00 |
| Total | 100 | 100 | 100 | 100 | 100 | 100 | 100 | 100 |

RESULTS/APPEARANCE

| | | | | | | | | |
|---|---|---|---|---|---|---|---|---|
| QUALITY | E | G | E | G | E | G | E | G+ |
| COLOR | W | W | W | W | W | W | W | W |
| ODOR | N.O | N.O | N.O | N.O | N.O | N.O | N.O | N.O |
| SHAKABILITY | Yes | Yes | Yes | Yes | Yes | Yes | Yes | Yes |

B

| | | | | | | | | |
|---|---|---|---|---|---|---|---|---|
| PEG 400 | 99.00 | 95.00 | 98.00 | 98.00 | 95.00 | 99.00 | 98.00 | 99.00 |
| Hydroxypropyl | | | | | | | | |
| Steareth 2 | | | | | | | | |
| Ketoconazole | 1.00 | | | | | | | |
| Minoxidil | | 5.00 | | | | | | |
| Mupirocin | | | 2.00 | | | | | |
| Nifedipine regular | | | | 2.00 | | | | |
| Permethrin BPC | | | | | 5.00 | | | |
| (cis:trans 25:75) | | | | | | | | |
| Piroxicam | | | | | | 1.00 | | |
| Salicylic acid | | | | | | | 2.00 | |
| Terbinafine HCl | | | | | | | | 1.00 |
| Total | 100 | 100 | 100 | 100 | 100 | 100 | 100 | |

RESULTS

| | | | | | | | | |
|---|---|---|---|---|---|---|---|---|
| QUALITY | G | G | G | E | E | E | E | E |
| COLOR | W | W | W | Light | W | Light | W | W |
| ODOR | N.O | N.O | N.O | N.O | N.O | N.O | N.O | N.O |
| SHAKABILITY | Yes | Yes | Yes | Yes | Yes | Yes | Yes | Yes |

Comments: formulations based on PEG-400, polymeric agent and a surfactant, produced good (G) to excellent (E), white (W) to light yellow, No odor (N.O.) and shakable foams.

‘Shakability’ means that the composition contains sufficient flow to allow the composition to be mixed or remixed on shaking. That is, it has fluid properties.

Note: The propellant can be added at a concentration of about 3% to about 25% or more.

### EXAMPLE 6

### Hydrophilic PG Containing Compositions with Various Active Agents

Stock Solution:

| | |
|---|---|
| Propylene glycol | 91.00 |
| Stearyl alcohol | 2.00 |
| Klucel EF | 2.00 |
| Laureth-4 | 2.00 |
| Glyceryl Monostearate/ | 3.00 |
| PEG 100 Stearate | |
| Total | 100.00 |

A

| | | | | | | | | |
|---|---|---|---|---|---|---|---|---|
| Propylene | 95.00 | 85.00 | 95.00 | 99.88 | 95.00 | 99.995 | 98.00 | 98.00 |
| glycol | | | | | | | | |
| Stearyl | | | | | | | | |
| alcohol | | | | | | | | |
| Klucel EF | | | | | | | | |
| Laureth-4 | | | | | | | | |
| Glyceryl | | | | | | | | |
| Monostearate/ | | | | | | | | |
| PEG 100 | | | | | | | | |
| Stearate | | | | | | | | |
| Acyclovir | 5.00 | | | | | | | |
| Azelaic acid | | 15.00 | | | | | | |
| Benzoyl | | | 5.00 | | | | | |
| peroxide | | | | | | | | |
| Betamethasone | | | | 0.12 | | | | |
| 17 valerate | | | | | | | | |
| micronized | | | | | | | | |
| Caffeine | | | | | 5.00 | | | |
| Calcipotriol | | | | | | 0.005 | | |
| hydrate | | | | | | | | |
| Ciclopiroxolamine | | | | | | | 2.00 | |
| Diclofenac | | | | | | | | 1.00 |
| sodium | | | | | | | | |
| Total | 100 | 100 | 100 | 100 | 100 | 100 | 100 | 100 |

RESULTS/APPEARANCE

| | | | | | | | | |
|---|---|---|---|---|---|---|---|---|
| QUALITY | E | G | E | G | E | G | E | E |
| COLOR | O.W | W | W | W | W | W | W | W |
| ODOR | N.O | N.O | N.O | N.O | N.O | N.O | N.O | N.O |
| SHAKABILITY | Yes | Yes | Yes | Yes | Yes | Yes | Yes | Yes |

B

| | | | | | | | | |
|---|---|---|---|---|---|---|---|---|
| Propylene glycol | 99.00 | 95.00 | 98.00 | 98.00 | 95.00 | 99.00 | 98.00 | 99.00 |
| Stearyl alcohol | | | | | | | | |
| Klucel EF | | | | | | | | |
| Laureth-4 | | | | | | | | |
| Glyceryl | | | | | | | | |
| Monostearate/ | | | | | | | | |
| PEG 100 Stearate | | | | | | | | |
| Ketoconazole | 1.00 | | | | | | | |
| Minoxidil | | 5.00 | | | | | | |
| Mupirocin | | | 2.00 | | | | | |
| Nifedipine regular | | | | 2.00 | | | | |
| Permethrin BPC | | | | | 5.00 | | | |
| (cis:trans 25:75) | | | | | | | | |
| Piroxicam | | | | | | 1.00 | | |
| Salicylic acid | | | | | | | 2.00 | |
| Terbinafine HCl | | | | | | | | 1.00 |
| Total | 100 | 100 | 100 | 100 | 100 | 100 | 100 | 100 |

RESULTS/APPEARANCE

| | | | | | | | | |
|---|---|---|---|---|---|---|---|---|
| QUALITY | E | G | G | G | E | E | E | E |
| COLOR | W | W | W | Light | O.W | O.W | O.W | W |
| | | | | Yellow | | | | |
| ODOR | N.O | N.O | N.O | No | No | No | No | No |
| | | Odor | Odor | Odor | Odor | Odor | Odor | Odor |
| SHAKABILITY | Yes | Yes | Yes | Yes | Yes | Yes | Yes | Yes |

Comments: formulations based on Propylene glycol, polymeric agent a surfactant and co-surfactant, produced good (G) to excellent (E), white (W) to light yellow, No odor (N.O.) and shakable foams.

The co emulsifiers are non essential and can be omitted although some adjustment may be needed to the surfactant combination as will be appreciated by someone skilled in the art.

Note: The propellant can be added at a concentration of about 3% to about 25% or more.

### EXAMPLE 7

### Hydrophilic PG Containing Compositions with Another Solvent DMI and Various Active Agents

Stock Solution:

| | |
|---|---|
| Propylene glycol | 46.00 |
| Glycerin anhydrous | 33.00 |
| Stearyl alcohol | 1.00 |
| Hydroxypropyl cellulose | 1.50 |
| Laureth-4 | 2.00 |
| Glyceryl Monostearate/ | 1.50 |
| PEG 100 Stearate | |
| Dimethyl isosorbide | 15.00 |
| Total | 100.00 |

A

| | | | | | | | | |
|---|---|---|---|---|---|---|---|---|
| Propylene | 95.00 | 85.00 | 95.00 | 99.88 | 95.00 | 99.995 | 98.00 | 98.00 |
| glycol | | | | | | | | |
| Glycerin | | | | | | | | |
| anhydrous | | | | | | | | |
| Stearyl alcohol | | | | | | | | |
| Hydroxypropyl | | | | | | | | |
| cellulose | | | | | | | | |
| Laureth-4 | | | | | | | | |
| Glyceryl | | | | | | | | |
| Monostearate/ | | | | | | | | |
| PEG 100 | | | | | | | | |
| Stearate | | | | | | | | |
| Dimethyl | | | | | | | | |
| isosorbide | | | | | | | | |
| Acyclovir | 5.00 | | | | | | | |
| Azelaic acid | | 15.00 | | | | | | |
| Benzoyl | | | 5.00 | | | | | |
| peroxide | | | | | | | | |
| Betamethasone | | | | 0.12 | | | | |
| 17 valerate | | | | | | | | |
| micronized | | | | | | | | |
| Caffeine | | | | | 5.00 | | | |
| Calcipotriol | | | | | | 0.005 | | |
| hydrate | | | | | | | | |
| Ciclopiroxol- | | | | | | | 2.00 | |
| amine | | | | | | | | |
| Diclofenac | | | | | | | | 1.00 |
| sodium | | | | | | | | |
| Total | 100 | 100 | 100 | 100 | 100 | 100 | 100 | 100 |

RESULTS/APPEARANCE

| | | | | | | | | |
|---|---|---|---|---|---|---|---|---|
| QUALITY | G | G | E | G | E | G | G | G |
| COLOR | O.W | W | W | W | W | W | W | W |
| ODOR | N.O | N.O | N.O | N.O | N.O | N.O | N.O | N.O |
| SHAKABILITY | Yes | Yes | Yes | Yes | Yes | Yes | Yes | Yes |

B

| | | | | | | | | |
|---|---|---|---|---|---|---|---|---|
| Propylene glycol | 99.00 | 95.00 | 98.00 | 98.00 | 95.00 | 99.00 | 98.00 | 99.00 |
| Glycerin | | | | | | | | |
| anhydrous | | | | | | | | |
| Stearyl alcohol | | | | | | | | |
| Hydroxypropyl | | | | | | | | |
| cellulose | | | | | | | | |
| Laureth-4 | | | | | | | | |
| Glyceryl | | | | | | | | |
| Monostearate/ | | | | | | | | |
| PEG 100 | | | | | | | | |
| Stearate | | | | | | | | |
| Dimethyl | | | | | | | | |
| isosorbide | | | | | | | | |
| Ketoconazole | 1.00 | | | | | | | |
| Minoxidil | | 5.00 | | | | | | |
| Mupirocin | | | 2.00 | | | | | |
| Nifedipine | | | | 2.00 | | | | |
| regular | | | | | | | | |
| Permethrin BPC | | | | | 5.00 | | | |
| (cis:trans 25:75) | | | | | | | | |
| Piroxicam | | | | | | 1.00 | | |
| Salicylic acid | | | | | | | 2.00 | |
| Terbinafine HCl | | | | | | | | 1.00 |
| Total | 100 | 100 | 100 | 100 | 100 | 100 | 100 | 100 |

RESULTS/APPEARANCE

| | | | | | | | | |
|---|---|---|---|---|---|---|---|---|
| QUALITY | G | G | G | G | E | E | E | G |
| COLOR | W | W | W | Light | O.W | O.W | O.W | W |
| | | | | Yellow | | | | |
| ODOR | N.O | N.O | N.O | N.O | N.O | N.O | N.O | N.O |
| SHAKABILITY | Yes | Yes | Yes | Yes | Yes | Yes | Yes | Yes |

Comments: formulations based on Propylene glycol, polymeric agent, a solvent, a surfactant and co-surfactant, produced good (G) to excellent (E), white (W) to light yellow, No odor (N.O.) and shakable foams.

The co emulsifiers are non essential and can be omitted although some adjustment may be needed to the surfactant combination as will be appreciated by someone skilled in the art.

Note: The propellant can be added at a concentration of about 3% to about 25% or more.

### EXAMPLE 8

### Hydrophilic PEG 400/200 Mixture Containing Compositions with Another Polymeric (Gelling) Agent a Silicone Mixture and Various Active Agents

| | | |
|---|---|---|
| PEG 400 | 40.38 | 38.50 |
| PEG 200 | 39.50 | 39.50 |
| DIMETHICONE | 3.00 | 3.00 |
| Cyclomethicone | 1.50 | 1.50 |
| ASOS | 3.00 | 3.00 |
| Stearic acid | 9.00 | 9.00 |
| Steareth-2 | 3.50 | 3.50 |
| Betamethasone 17 valerate | 0.12 | |
| micronized | | |
| Mupirocin | | 2.00 |
| total | 100.00 | 100.00 |

RESULTS/APPEARANCE

| | | |
|---|---|---|
| QUALITY | Good | Good |
| COLOR | White | White |
| ODOR | No Odor | No Odor |
| SHAKABILITY | Yes | Yes |

Comments: formulations based on polyethylene glycol, surfactant, polymeric agent and a silicone, produced good (G) white (W), No odor (N.O.) and shakable foams.

Note: The propellant can be added at a concentration of about 3% to about 25% or more.

### EXAMPLE 9

### Hydrophilic Propylene Glycol Containing Compositions with Another Polymeric (Gelling) Agent and Various Active Agents

| | | | | |
|---|---|---|---|---|
| PROPYLENE GLYCOL | 97.38 | 95.50 | 92.50 | 82.50 |
| Steareth-2 | 2.00 | 2.00 | 2.00 | 2.00 |
| CARBOMER 934 | 0.50 | 0.50 | 0.50 | 0.50 |
| Betamethasone 17 | 0.12 | | | |
| valerate micronized | | | | |
| Mupirocin | | 2.00 | | |
| Minoxidil | | | 5.00 | |
| Azelaic acid | | | | 15.00 |
| total | 100.00 | 100.00 | 100.00 | 100.00 |

RESULTS/APPEARANCE

| | | | | |
|---|---|---|---|---|
| QUALITY | Good | Good | Good | Good |
| COLOR | White | White | White | White |
| ODOR | No Odor | No Odor | No Odor | No Odor |
| SHAKABILITY | Good | Good | Good | Good |

Comments: formulations based on Propylene glycol, polymeric agent and a surfactant, produced good (G) white (W) No odor (N.O.) and shakable foams.

Note: The propellant can be added at a concentration of about 3% to about 25% or more.

### EXAMPLE 10

### Foamable Hygroscopic Composition Containing Polyethylene Glycol with No surfactant

% w/w

| | |
|---|---|
| PEG 400 | 93.50 |
| Klucel GF | 0.50 |
| Propellant (Butane/propane) | 6.00 |
| Foam quality | E |
| Density | 0.09 |

### EXAMPLE 9

### Comparison Between Polyethylene-Based Foamable Compositions with and without Gelling Agent

The present example clarifies the need of a gelling agent in the composition, in order to provide improved usability. The compositions of the test articles are provided in the following table. All foams were dispensed on a warm surface (38° C.), and the time to full collapse of the foam was measured. As shown in the table, it has been strikingly demonstrated that foam compositions without a gelling agent exhibit a 100% breakdown within 30 seconds, while foams containing gelling agent remained, with and without surfactant, were stable for several minutes. his is relevant from the usability point of view, since a foam that is unstable at skin temperature cannot be applied to large areas effectively.

| | | | | | Formulation with | |
|---|---|---|---|---|---|---|
| | Formulations without gelling agent | | | | gelling agent | |
| | | PG34 | PG35 | PG36 | TEC49 | PG29 |
| | PG33 % w/w | % w/w | % w/w | % w/w | % w/w | % w/w |
| PEG 400 | 87.25 | 93.00 | 91.00 | 92.00 | 90.50 | 93.50 |
| Klucel GF (gelling agent) | — | — | — | — | 0.50 | 0.50 |
| Ceteareth-16 | — | — | 2.00 | 1.00 | — | — |
| Emulsiying Wax NF | 1.80 | — | — | — | — | — |
| Steareth-10 | — | 0.40 | — | 0.50 | — | — |
| PEG-40 stearate | 1.35 | — | — | — | — | — |
| Steareth-2 | — | 0.60 | 1.00 | 0.50 | 1.00 | |
| Span 60 | 2.70 | — | — | — | — | — |
| Polysorbate 60 | 0.90 | — | — | — | — | — |
| Propellant | 6.00 | 6.00 | 6.00 | 6.00 | 8.00 | 6.00 |
| Collapse time (Seconds; | <30 | <30 | <30 | <30 | 240 | >300 |
| 38° C.) | | | | | | |

What is claimed is:

1. A foamable pharmaceutical or cosmetic composition, comprising:
a solvent comprising polyethylene glycol (PEG) or PEG derivative and mixtures thereof, wherein the PEG or PEG derivative is present at a concentration of about 70% to about 96.5% by weight of the total composition;
a surface-active agent at a concentration of about 0.1% to less than about 10% by weight of the total composition; and
a therapeutically effective amount of at least one active agent.

2. The composition of claim 1, further comprising:
at least one gelling agent at a concentration of about 0.1% to about 5% by weight of the total composition.

3. The composition of claim 2, wherein the at least one gelling agent is selected from the group consisting of natural polymeric materials, semi-synthetic polymeric materials, synthetic polymeric materials, inorganic gelling agents and mixtures thereof.

4. The composition of claim 2 wherein the gelling agent also has surface active agent properties.

5. The composition of claim 3, wherein the at least one gelling agent is a semi-synthetic polymeric material.

6. The composition of claim 5, wherein the semi-synthetic polymeric material is a cellulose ether.

7. The composition of claim 1, further comprising at least one liquefied or compressed gas propellant, at a concentration of about 3% to about 25% by weight of the total composition.

8. The composition of claim 1, wherein the composition is contained within a pressurized container.

9. The composition of claim 1, wherein the polyethylene glycol (PEG) or PEG derivative is liquid or is flowable at ambient temperature.

10. The composition of claim 1, wherein the polyethylene glycol (PEG) or PEG derivative has an average Molecular Weight ranging from about 190 kD to about 10,000 kD.

11. The composition of claim 1, wherein the polyethylene glycol (PEG) or PEG derivative is selected from the group consisting of PEG200, PEG300, PEG400, PEG600, PEG 4000, PEG 6000, PEG 10000 and mixtures thereof.

12. The composition of claim 1, wherein the polyethylene glycol (PEG) or PEG derivative is PEG400.

13. The composition of claim 1, wherein the surface-active agent is a non-ionic surface-active agent.

14. The composition of claim 1, wherein the surface-active agent is selected from the group consisting of sorbitan derivatives, alkoxylated alcohols, hydroxylated derivatives of polymeric silicones, alkylated derivatives of hydroxylated polymeric silicones, glyceryl esters, beeswax derivatives, lecithin and mixtures thereof.

15. The composition of claim 1, wherein the surface-active agent is selected from the group consisting of polysorbates, polyoxyethylene fatty acid esters, polyoxyethylene alkyl ethers, sucrose esters, partial esters of sorbitol and its anhydrides, fatty alcohols, fatty acids, mono and diglycerides, isoceteth-20, sodium methyl cocoyl taurate, sodium methyl oleoyl taurate, sodium lauryl sulfate, triethanolamine lauryl sulfate and betaines.

16. The composition of claim 14, wherein the surface-active agent is a glyceryl ester.

17. The composition of claim 14, wherein the surface-active agent is an alkoxylated alcohol.

18. The composition of claim 15, wherein the surface-active agent is a polyoxyethylene alkyl ether.

19. The composition of claim 15, wherein the surface-active agent is a polyoxyethylene fatty acid ester.

20. The composition of claim 1, further comprising a mixture of a non-ionic surface-active agent and an ionic surface-active agent.

21. The composition of claim 1, further comprising a foam adjuvant selected from the group consisting of fatty alcohols having greater than or equal to 15 carbons and fatty acids having greater than or equal to 16 carbons.

22. The composition of claim 1 further comprising at least one other solvent selected from the group consisting of polyols, glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, dimethylsulfoxide (DMSO), dimethylformanide, methyl dodecyl sulfoxide, dimethylacetamide; monooleate of ethoxylated glycerides (with 8 to 10 ethylene oxide units); azone (1-dodecylazacycloheptan-2-one), 2-(n-nonyl)-1,3-dioxolane; isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; acetamide; triolein; alkanoic acids, caprylic acid; lactam compounds, azone; alkanols, dialkylamino acetates, polyethylene glycol (PEG) or PEG derivative and mixtures thereof.

23. The composition of claim 1 further comprising at least one other solvent selected from the group consisting of propylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, dimethyl isosorbide, glycofurol and ethoxydiglycol (transcutol).

24. The composition of claim 22 wherein the at least one other solvent is glycerol (glycerin).

25. The composition of claim 22, wherein the at least one other solvent is propylene glycol.

26. The composition of claim 1, wherein the composition contains less than about 5% of a lower alcohol having up to 5 carbon atoms in its carbon chain skeleton.

27. The composition of claim 1, wherein the composition contains no water.

28. The composition of claim 1, wherein the composition contains substantially no water.

29. The composition of claim 1, wherein the composition contains less than about 10% of water by weight of the total composition.

30. The composition of claim 1, wherein the composition contains less than about 20% of water by weight of the total composition.

31. The composition of claim 1, wherein the composition contains less than about 30% of water by weight of the total composition.

32. The composition of claim 1, wherein the composition has a specific gravity of about 0.01 g/ml to about 0.3 g/mL upon release from the pressurized container.

33. The composition of claim 1, further comprising an antioxidant.

34. The composition of claim 33, wherein the antioxidant is selected from the group consisting of ascorbic acid (vitamin C) and its salts, ascorbyl esters of fatty acids, ascorbic acid derivatives, tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, lipoic acid, amines, sulfhydryl compounds, dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

35. The composition of claim 1, further comprising an emollient.

36. The composition of claim 35, wherein the emollient is selected from the group consisting of hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

37. The composition of claim 1, further comprising a buffering agent.

38. The composition of claim 1, further comprising a chelating agent.

39. The composition of claim 1, wherein the at least one active agent is selected from the group consisting of an anti-infective, an antibiotic, an antibacterial agent, an antifungal agent, an antiviral agent, an anti-parasitic agent, an anti-inflammatory agent, an immunosuppressive agent, and immunomodulator, an immuno regulating agent, an anesthetic, an analgesic, an anti-allergic agent, a corticosteroid, a non-steroidal anti-inflammatory agent, a retinoid, a keratolytic agent, an anti-proliferative agent, an anticancer agent, a photodynamic therapy agent, an anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

40. The composition of claim 1, wherein the at least one active agent is selected from the group consisting of an anti-inflammatory agent, an antinfective agent, a keratolytically active agent, a vasoactive agent and a retinoid.

41. The composition of claim 1, wherein the at least one active agent is selected from the group consisting of a corticosteroid, a non steroid anti-inflammatory agent, an anti-bacterial agent, a keratolytically active agent, a vasoactive agent and a retinoid.

42. The composition of claim 1, wherein the at least one active agent is vitamin.

43. The composition of claim 41, wherein the corticosteriod is selected from the group consisting of Clobetasol proprionate, Halobetasol proprionate, Betamethasone diproprionate, Betamethasone valerate, Fluocinolone acetonide, Halcinonide, Betamethasone valerate, Fluocinolone acetonide, Hydrocortisone valerate, Triamcinolone acetonide and Hydrocortisone.

44. The composition of claim 41, wherein the non steroid anti-inflammatory active agent is selected from the group consisting of:
oxicams, piroxicam, isoxicam, tenoxicam, and sudoxicam;
salicylates, salicylic acid, ethyl salicylate, methyl salycilate, aspirin, disalcid, benorylate, trilisate, safapryn, solprin, diflunisal, and fendosal;
acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
fenamates, mefenamic, meclofenamic, flufenamic, niflumic, and tolfenamic acids;
propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
pyrazoles, phenylbutazone, oxyphenbutazone, feprazone, azapropazone, and trimethazone.

45. The composition of claim 41, wherein the anti-bacterial agent is selected from the group consisting of macrolide antibiotics, erythromycin; sulfonamide (in its base form), sulfanilamide, sulfadiazine and sulfacetamide; mupirocin; tetracyclines, tetracycline and doxycycline; specific oil-soluble species of synthetic and semi-synthesic penicillins and beta-lactams; cloramphenicol; specific oil-soluble species of imidazoles; dicarboxylic acids, such as azelaic acid; salicylates; peptide antibiotics; cyclic peptides, such as cyclosporine, tacrolimus, pimecrolimus and sirolimus (rapamycin); and non-specific antibacterial agents such as strong oxidants and free radical liberating compounds, bleaching agents, iodine compounds and benzoyl peroxide.

46. The composition of claim 41, wherein the keratolytically active agent is selected from the group consisting of phenol and substituted phenolic compounds; dihydroxy benzene and derivatives; resorcinol (m-dihydroxybenzene) and derivatives; hydroquinone (p-dihydroxybenzene); cresols; vitamin A and its derivatives, such as retinoic acid, isoretinoic acid, retinol and retinal; alpha-hydroxy acids, such as lactic acid and glycolic acid and their respective salts and derivatives; beta-hydroxy acids, such as Salicylic acid (o-hydroxybenzoic acid) and its salts and pharmaceutically acceptable derivatives; and urea and its derivatives.

47. The composition of claim 41, wherein the vasoactive agent is selected from the group consisting of minoxidil, sildenafil and caffeine.

48. The composition of claim 41, wherein the retinoid agent is selected from the group consisting of retinol, retinal, all trans retinoic acid and derivatives, etretinate, actiretin, isotretinoin, adapalene and tazarotene and isomers and analogs thereof.

49. The composition of claim 41, wherein the at least one active agent is selected from the group consisting of Acyclovir, Azelaic acid, Benzoyl peroxide, Betamethasone 17 valerate micronized, Caffeine, Calcipotriol hydrate, Ciclopiroxolamine, Diclofenac sodium, Ketoconazole, Miconazole nitrate, Minoxidil, Mupirocin, Nifedipine regular, Permethrin BPC (cis:trans 25:75), Piroxicam, Salicylic acid and Terbinafine HCl.

50. The composition of claim 1, wherein the active agent is selected for the treatment of a disorder of the skin, mucosal membrane, ear channel, vagina, penile urethra, colon and rectum.

51. The composition of claim 1, wherein the active agent is administered via transdermal delivery.

52. The composition of claim 1, wherein the at least one active agent is of solid matter.

53. The composition of claim 1, wherein the at least one active agent is soluble in the composition.

54. The composition of claim 1, having the properties of breakable foam for treating, alleviating or preventing a dermatological or mucosal disorder.

55. The composition of claim 1 wherein a component of the foamable composition selected from a potent solvent, a co-solvent, a surface-active agent, a gelling agent, an emollient and a foam adjuvant may itself contribute to the pharmaceutical or cosmetic effect of the composition.

56. A foamable pharmaceutical or cosmetic carrier composition for dermatological use, comprising:
a solvent comprising polyethylene glycol (PEG) or PEG derivative and mixtures thereof, wherein the PEG or PEG derivative is present at a concentration of about 70% to about 96.5% by weight of the total composition;
a surface-active agent at a concentration of about 0.1% to less than about 10% by weight of the total composition;
at least one gelling agent at a concentration of about 0.1% to about 5% by weight of the total composition; and
at least one liquefied or compressed gas propellant, at a concentration of about 3% to about 25% by weight of the total composition,
wherein the composition is stored in an aerosol container and upon release expands to form a breakable foam.

57. A composition, comprising:
a polyethylene glycol (PEG) or PEG derivative and mixtures thereof, wherein the PEG or PEG derivative is present at a concentration of about 70% to about 96.5% by weight of the total composition;
a surface-active agent at a concentration of about 0.1% to less than about 10% by weight of the total composition; and
a therapeutically effective amount of at least one agent selected from the group consisting of an antinfective agent, a keratolytically active agent, a vasoactive agent and a retinoid.

58. A composition, comprising:
a polyethylene glycol (PEG) or PEG derivative and mixtures thereof, wherein the PEG or PEG derivative is present at a concentration of about 70% to about 96.5% by weight of the total composition;
a surface-active agent at a concentration of about 0.1% to less than about 10% by weight of the total composition; and
a therapeutically effective amount of at least one anti-inflammatory or antiallergic agent, wherein said agent reduces the occurrence of pro-inflammatory cytokines and/or inhibits the effect of pro-inflammatory cytokines.

59. A method of treating, alleviating or preventing a dermatological, cosmetic or mucosal disorder, comprising administering topically to a subject having said disorder a therapeutically effective amount of a foamable composition according to any of claims 1, 56, 57 or 58.

60. The method of claim 59 wherein the disorder is selected from the group consisting of an inflammatory disorder, an infection, dermatoses, keratosis, hyperkeratinization and a vaso disorder.

61. A foamable pharmaceutical or cosmetic composition, comprising:
a solvent comprising propylene glycol, wherein the propylene glycol is present at a concentration of about 70% to about 96.5% by weight of the total composition;
a surface-active agent at a concentration of about 0.1% to less than about 10% by weight of the total composition; and
a therapeutically effective amount of at least one active agent.

62. The composition of claim 61, further comprising:
at least one gelling agent at a concentration of about 0.1% to about 5% by weight of the total composition.

63. The composition of claim 62, wherein the at least one gelling agent is selected from the group consisting of natural polymeric materials, semi-synthetic polymeric materials, synthetic polymeric materials, inorganic gelling agents and mixtures thereof.

64. The composition of claim 62 wherein the gelling agent also has surface active agent properties.

65. The composition of claim 63, wherein the at least one gelling agent is a semi-synthetic polymeric material.

66. The composition of claim 65, wherein the semi-synthetic polymeric material is a cellulose ether.

67. The composition of claim 61, further comprising at least one liquefied or compressed gas propellant, at a concentration of about 3% to about 25% by weight of the total composition.

68. The composition of claim 61, wherein the composition is contained within a pressurized container.

69. The composition of claim 61, wherein the surface-active agent is a non-ionic surface-active agent.

70. The composition of claim 61, wherein the surface-active agent is selected from the group consisting of sorbitan derivatives, alkoxylated alcohols, hydroxylated derivatives of polymeric silicones, alkylated derivatives of hydroxylated polymeric silicones, glyceryl esters, beeswax derivatives, lecithin and mixtures thereof.

71. The composition of claim 61, wherein the surface-active agent is selected from the group consisting of polysorbates, polyoxyethylene fatty acid esters, polyoxyethylene alkyl ethers, sucrose esters, partial esters of sorbitol and its anhydrides, fatty alcohols, fatty acids, mono and diglycerides, isoceteth-20, sodium methyl cocoyl taurate, sodium methyl oleoyl taurate, sodium lauryl sulfate, triethanolamine lauryl sulfate and betaines.

72. The composition of claim 70, wherein the surface-active agent is a glyceryl ester.

73. The composition of claim 70, wherein the surface-active agent is an alkoxylated alcohol.

74. The composition of claim 71, wherein the surface-active agent is a polyoxyethylene alkyl ether.

75. The composition of claim 71, wherein the surface-active agent is a polyoxyethylene fatty acid ester.

76. The composition of claim 61, further comprising a mixture of a non-ionic surface-active agent and an ionic surface-active agent.

77. The composition of claim 61, further comprising a foam adjuvant selected from the group consisting of fatty alcohols having greater than or equal to 15 carbons and fatty acids having greater than or equal to 16 carbons.

78. The composition of claim 61, further comprising at least one other solvent selected from the group consisting of polyols, glycerol (glycerin), hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane; isopropyl myristate/palmitate, ethyl acetate, butyl acetate, methyl proprionate, capric/caprylic triglycerides, octylmyristate, dodecyl-myristate, myristyl alcohol, lauryl alcohol, lauric acid, lauryl lactate ketones, acetamide, triolein, alkanoic acids, caprylic acid, lactam compounds, alkanols, dialkylamino acetates, polyethylene glycol (PEG) or PEG derivative and mixtures thereof.

79. The composition of claim 61 further comprising at least one other solvent selected from the group consisting of polyethylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, dimethyl isosorbide, glycofurol and ethoxydiglycol (transcutol).

80. The composition of claim 78 wherein the at least one other solvent is glycerol (glycerin).

81. The composition of claim 78, wherein the at least one other solvent is polyethylene glycol (PEG) or PEG derivative and mixtures thereof.

82. The composition of claim 81, wherein the polyethylene glycol (PEG) or PEG derivative and mixtures thereof is liquid or is flowable at ambient temperature.

83. The composition of claim 81, wherein the polyethylene glycol (PEG) or PEG derivative and mixtures thereof have an average Molecular Weight ranging from about 190 kD to about 10,000 kD.

84. The composition of claim 81, wherein the polyethylene glycol (PEG) or PEG derivative is selected from the group consisting of PEG200, PEG300, PEG400, PEG600, PEG 4000, PEG 6000 and PEG 10000 and mixtures thereof.

85. The composition of claim 81, wherein the polyethylene glycol (PEG) or PEG derivative is PEG400.

86. The composition of claim 61, comprising less than about 5% of a lower alcohol having up to 5 carbon atoms in its carbon chain skeleton.

87. The composition of claim 61, wherein the composition contains no water.

88. The composition of claim 61, wherein the composition contains substantially no water.

89. The composition of claim 61, wherein the composition contains less than about 10% of water by weight of the total composition.

90. The composition of claim 61, wherein the composition contains less than about 20% of water by weight of the total composition.

91. The composition of claim 61, wherein the composition contains less than about 30% of water by weight of the total composition.

92. The composition of claim 61, wherein the composition has a specific gravity of about 0.01 g/mL to about 0.3 g/mL upon release from the pressurized container.

93. The composition of claim 61, further comprising an antioxidant.

94. The composition of claim 93, wherein the antioxidant is selected from the group consisting of ascorbic acid (vitamin C) and its salts, ascorbyl esters of fatty acids, ascorbic acid derivatives, tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, lipoic acid, amines, sulfhydryl compounds, dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

95. The composition of claim 61, further comprising an emollient.

96. The composition of claim 95, wherein the emollient is selected from the group consisting of hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

97. The composition of claim 61, further comprising a buffering agent.

98. The composition of claim 61, further comprising a chelating agent.

99. The composition of claim 61, wherein the at least one active agent is selected from the group consisting of an anti-infective, an antibiotic, an antibacterial agent, an antifungal agent, an antiviral agent, an anti-parasitic agent, an anti-inflammatory agent, an immunosuppressive agent, and immunomodulator, an immuno regulating agent, an anesthetic, an analgesic, an anti-allergic agent, a corticosteroid, a non-steroidal anti-inflammatory agent, a retinoid, a keratolytic agent, an anti-proliferative agent, an anticancer agent, a photodynamic therapy agent, an anti-wrinkle agent, a radical scavenger, a self-tanning agent, a skin whitening agent, a skin protective agent, an anti-cellulite agent, a massaging oil and an anti-wart agent, a refatting agent, a lubricating agent and mixtures thereof.

100. The composition of claim 61, wherein the at least one active agent is selected from the group consisting of an anti-inflammatory agent, an antinfective agent, a keratolytically active agent, a vasoactive agent and a retinoid.

101. The composition of claim 61, wherein the at least one active agent is selected from the group consisting of a, corticosteroid, an a non steroid anti-inflammatory agent, an anti-bacterial agent, a keratolytically active agent, a vasoactive agent and a retinoid.

102. The composition of claim 61, wherein the at least one active agent is vitamin.

103. The composition of claim 101, wherein the corticosteriod is selected from the group consisting of Clobetasol proprionate, Halobetasol proprionate, Betamethasone diproprionate, Betamethasone valerate, Fluocinolone acetonide, Halcinonide, Betamethasone valerate, Fluocinolone acetonide, Hydrocortisone valerate, Triamcinolone acetonide, and Hydrocortisone.

104. The composition of claim 101, wherein the non steroid anti-inflammatory active agent is selected from the group consisting of:
oxicams, piroxicam, isoxicam, tenoxicam, and sudoxicam;
salicylates, salicylic acid, ethyl salicylate, methyl salycilate, aspirin, disalcid, benorylate, trilisate, safapryn, solprin, diflunisal, and fendosal;
acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
fenamates, mefenamic, meclofenamic, flufenamic, niflumic, and tolfenamic acids;
propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
pyrazoles, phenylbutazone, oxyphenbutazone, feprazone, azapropazone, and trimethazone.

105. The composition of claim 101, wherein the anti-bacterial agent is selected from the group consisting of macrolide antibiotics, erythromycin; sulfonamide (in its base form), sulfanilamide, sulfadiazine and sulfacetamide; mupirocin; tetracyclines, tetracycline and doxycycline; specific oil-soluble species of synthetic and semi-synthesic penicillins and beta-lactams; cloramphenicol; specific oil-soluble species of imidazoles; dicarboxylic acids, such as azelaic acid; salicylates; peptide antibiotics; cyclic peptides, such as cyclosporine, tacrolimus, pimecrolimus and sirolimus (rapamycin); and non-specific antibacterial agents such as strong oxidants and free radical liberating compounds, bleaching agents, iodine compounds and benzoyl peroxide.

106. The composition of claim 101, wherein the keratolytically active agent is selected from the group consisting of—phenol and substituted phenolic compounds; dihydroxy benzene and derivatives; resorcinol (m-dihydroxybenzene) and derivatives; hydroquinone (p-dihydroxybenzene); cresols; vitamin A and its derivatives, such as retinoic acid, isoretinoic acid, retinol and retinal; alpha-hydroxy acids, such as lactic acid and glycolic acid and their respective salts and derivatives; beta-hydroxy acids, such as Salicylic acid (o-hydroxybenzoic acid) and its salts and pharmaceutically acceptable derivatives; and urea and its derivatives.

107. The composition of claim 101, wherein the vasoactive agent is selected from the group consisting of minoxidil, sildenafil and caffeine.

108. The composition of claim 101, wherein the at retinoid agent is selected from the group consisting of retinol, retinal, all trans retinoic acid and derivatives; etretinate, actiretin, isotretinoin, adapalene and tazarotene and isomers and analogs thereof.

109. The composition of claim 101, wherein the at least one active agent is selected from the group consisting of: Acyclovir, Azelaic acid, Benzoyl peroxide, Betamethasone 17 valerate micronized, Caffeine, Calcipotriol hydrate, Ciclopiroxolamine, Diclofenac sodium, Ketoconazole, Miconazole nitrate, Minoxidil, Mupirocin, Nifedipine regular, Permethrin BPC (cis:trans 25:75), Piroxicam, Salicylic acid and Terbinafine HCl.

110. The composition of claim 61, wherein the active agent is selected for the treatment of a disorder of the skin, mucosal membrane, ear channel, vagina, penile urethra, colon and rectum.

111. The composition of claim 61, wherein the active agent is administered via transdermal delivery.

112. The composition of claim 61, wherein the at least one active agent is of solid matter.

113. The composition of claim 61, wherein the at least one active agent is soluble in the composition.

114. The composition of claim 61, having the properties of breakable foam for treating, alleviating or preventing a dermatological or mucosal disorder.

115. The composition of claim 61 wherein a component of the foamable composition selected from a potent solvent, a co-solvent, a surface-active agent, a gelling agent, an emollient and a foam adjuvant may itself contribute to the pharmaceutical or cosmetic effect of the composition.

116. A foamable pharmaceutical or cosmetic carrier composition for dermatological use, comprising:
a solvent comprising propylene glycol, wherein the propylene glycol is present at a concentration of about 70% to about 96.5% by weight of the total composition;
a surface-active agent at a concentration of about 0.1% to less than about 10% by weight of the total composition;
at least one gelling agent at a concentration of about 0.1% to about 5% by weight of the total composition; and
at least one liquefied or compressed gas propellant, at a concentration of about 3% to about 25% by weight of the total composition,
wherein the composition is stored in an aerosol container and upon release expands to form a breakable foam.

117. A composition, comprising:
a solvent comprising propylene glycol, wherein the propylene glycol is present at a concentration of about 70% to about 96.5% by weight of the total composition;
a surface-active agent at a concentration of about 0.1% to less than about 10% by weight of the total composition; and
a therapeutically effective amount of at least one active agent selected from the group consisting of an antinfective agent, a keratolytically active agent, a vasoactive agent and a retinoid.

118. A composition, comprising:
a solvent comprising propylene glycol, wherein the propylene glycol is present at a concentration of about 70% to about 96.5% by weight of the total composition;
a surface-active agent at a concentration of about 0.1% to less than about 10% by weight of the total composition; and
a therapeutically effective amount of at least one anti-inflammatory or antiallergic agent, wherein said agent reduces the occurrence of pro-inflammatory cytokines and/or inhibits the effect of pro-inflammatory cytokines.

119. A method of treating, alleviating or preventing a dermatological, cosmetic or mucosal disorder, comprising administering topically to a subject having said disorder a therapeutically effective amount of a potent solvent foam composition according to any of claims 61, 116, 117, or 118.

120. The method of claim 119 wherein the disorder is selected from the group consisting of an inflammatory disorder, an infection, dermatoses, keratosis, hyperkeratinization and a vaso disorder.

121. The composition of claim 1, wherein the surface-active agent is Mirj 52.
